(19) United States
(12) Patent Application Publication
(10) Pub. No.: US 2024/0093300 A1
(43) Pub. Date: Mar. 21, 2024

(54) METHYLATED DNA FRAGMENT ENRICHMENT, METHODS, COMPOSITIONS AND KITS

(71) Applicant: GRAIL, LLC, Menlo Park, CA (US)

(72) Inventors: Craig BETTS, Menlo Park, CA (US); Gordon CANN, Menlo Park, CA (US); Byoungsok JUNG, Menlo Park, CA (US); Nathan HUNKAPILLER, Menlo Park, CA (US)

(21) Appl. No.: 18/011,145

(22) PCT Filed: Jun. 20, 2021
(86) PCT No.: PCT/US2021/038161

§ 371 Date: Dec. 16, 2022

Related U.S. Application Data
(60) Provisional application No. 63/041,690, filed on Jun. 19, 2020.

(51) Int. Cl. C12Q 1/6886; C12Q 1/6874

(52) U.S. Cl. CPC C12Q 1/6886; C12Q 1/6874; C12Q 1/6806

(57) ABSTRACT

A method of processing an input sample, as well as related kits and compositions, is provided herein. In various instances, the disclosure relates to providing an input sample comprising nucleic acid fragments, wherein in at least a portion of the nucleic acid fragments each fragment comprises one or more methylated cytosines; converting unmethylated cytosines of nucleic acid fragments of the input sample to uracils, yielding converted fragments; copying the converted fragments using a mixture of nucleotides, the mixture comprising a mixture of: binding moiety-modified cytosines and binding moiety-lacking cytosines; binding moiety-modified guanines and binding moiety-lacking guanines; or binding moiety-modified cytosines, binding moiety-lacking cytosines, binding moiety-modified guanines, and binding moiety-lacking guanines; wherein the copying yields a mixture of binding moiety-modified fragments and unmodified fragments which may be separated to provide a set of fragments enriched for hypermethylated fragments.

### 1. PRIORITY CLAIM

This application claims the benefit of U.S. Provisional Patent Application No. 63/041,690, “Enrichment for Methylated DNA Fragments, and Related Methods, Compositions and Kits” filed on Jun. 19, 2020.

### 2. FIELD OF THE INVENTION

The invention relates to methods, compositions and kits for enrichment of methylated DNA fragments.

### 3. BACKGROUND

Methylation of cytosines in DNA is an increasingly important diagnostic marker for a variety of diseases and conditions. DNA methylation profiling has been used as a diagnostic tool for detection, diagnosis, and/or characterization of cancer. These diagnostic analyses often use extracellular fragmented DNA from bodily fluids (cfDNA). In some cases, tests using cfDNA methylation markers may require identification of hypermethylated fragments of DNA using expensive techniques, such as NextGen sequencing. Moreover, tests may require sequencing of large numbers of targets and fragments to identify hypermethylated fragments. It is therefore desirable to provide sample preparation processes that enrich for methylated or hypermethylated fragments and thereby reduce the amount of DNA that is subject to subsequent processing, such as sequencing.

### 4. SUMMARY OF THE INVENTION

The disclosure provides methods of processing nucleic acid fragments. The methods may include providing an input sample including nucleic acid fragments, wherein in at least a portion of the nucleic acid fragments each fragment may include one or more methylated cytosines. The methods may include converting unmethylated cytosines of nucleic acid fragments of the input sample to uracils, yielding converted fragments. The methods may include copying the converted fragments using a mixture of nucleotides, the mixture including a mixture of binding moiety-modified cytosines and binding moiety-lacking cytosines; binding moiety-modified guanines and binding moiety-lacking guanines; or binding moiety-modified cytosines, binding moiety-lacking cytosines, binding moiety-modified guanines, and binding moiety-lacking guanines. The copying may yield a mixture of binding moiety-modified fragments and unmodified fragments. The methods may include binding at least some of the binding moiety-modified fragments to a substrate, yielding bound fragments and unbound supernatant fragments.

The mixture of nucleotides may include binding moiety-modified cytosines. The mixture of nucleotides may include binding moiety-modified guanines. The mixture of nucleotides may include binding moiety-modified cytosines and binding moiety-modified guanines.

The methods may include separating the bound fragments from the unbound supernatant fragments, yielding the bound fragments enriched for fragments with one or more methylated cytosines. The methods may include separating the bound fragments from the unbound supernatant fragments, yielding the bound fragments enriched for fragments with two or more methylated cytosines.

The input sample may be enriched for targets. The input sample may be enriched for targets prior to the converting step. The targets may be selected for a methylation assay. The targets may be selected for a methylation assay for cancer, cancer type, cancer tissue of origin, cancer stage, or combinations of the foregoing.

The input sample may be from a subject selected for diagnosis, disease characterization, or screening using a test assessing hypermethylated fragments. The input sample may include DNA isolated from a bodily fluid. The input sample may include DNA from a cfDNA sample. The input sample may include fragmented genomic DNA.

The converting may be accomplished by a methods including selectively deaminating the unmethylated cytosines. The converting may be accomplished by a methods including enzymatic conversion of the unmethylated cytosines to uracils.

The binding moiety-modified cytosines may include biotin-modified cytosines. The binding moiety-modified guanines may include biotin-modified guanines.

The substrate may, for example, include beads or wells.

The methods may yield bound fragments enriched for fragments with 2 and greater methylated cytosines. The methods may yield bound fragments enriched for fragments with 5 and greater methylated cytosines. The methods may yield bound fragments enriched for fragments with 10 and greater methylated cytosines.

Copying the fragments may include conducting a first primer extension reaction in the presence of the mixture of nucleotides. Copying the fragments may include conducting a second primer extension reaction in the presence of the mixture of nucleotides.

Providing the input sample may include obtaining from a sample, and including in the input sample, nucleic acid fragments potentially including multiple CpG sites. Providing the input sample may include obtaining from a sample, and including in the input sample, nucleic acid fragments potentially including 1 or more CpG sites. Providing the input sample may include obtaining from a sample, and including in the input sample, nucleic acid fragments potentially including 2 or more CpG sites. Providing the input sample may include obtaining from a sample, and including in the input sample, nucleic acid fragments potentially including 3 or more CpG sites. Providing the input sample may include obtaining from a sample, and including in the input sample, nucleic acid fragments hypermethylated in cancer samples relative to non-cancer samples. Providing the input sample may include obtaining from a sample, and including in the input sample, nucleic acid fragments hypermethylated in non-cancer samples relative to cancer samples. Providing the input sample may include obtaining from a sample, and including in the input sample, nucleic acid fragments hypermethylated in specific target tissues relative to other tissues.

The mixture of nucleotides may include from 1 to 20 percent binding moiety-modified cytosines with the remainder of the cytosines lacking the binding moiety. The mixture of nucleotides may include from 2.5 to 10 percent binding moiety-modified cytosines with the remainder of the cytosines lacking the binding moiety. The mixture of nucleotides may include from 1 to 20 percent binding moiety-modified guanines with the remainder of the guanines lacking the binding moiety. The mixture of nucleotides may include from 2.5 to 10 percent binding moiety-modified guanines with the remainder of the guanines lacking the binding moiety. The mixture of nucleotides may include from 1 to 20 percent binding moiety-modified cytosines and guanines with the remainder of the cytosines and guanines lacking the binding moiety. The mixture of nucleotides may include from 2.5 to 10 percent binding moiety-modified cytosines and guanines with the remainder of the cytosines and guanines lacking the binding moiety.

The separating may bound fragments enriched, relative to the input sample, for informative fragments for a methylation assay. The separating may yield bound fragments having a reduced content, relative to the input sample, of uninformative fragments for a methylation assay.

The methods may include eluting the bound fragments to yield a fragment library enriched, relative to the input sample, for informative fragments for a methylation assay. The methods may include eluting the bound fragments to yield a fragment library having a reduced content, relative to the input sample, of uninformative fragments for a methylation assay.

The methods may include preparing a sequencing library from the fragment library. The methods may include sequencing the sequencing library. The sequencing may be performed to a sequencing depth ranging from 5 to 20 million reads. The sequencing may be performed to a sequencing depth ranging from 5 to 15 million reads. The sequencing may be performed to a sequencing depth ranging from 5 to 15 million reads.

The disclosure provides methods of making a composition, the methods may include combining adenines, thymines, cytosines and guanines to produce the composition. The cytosines may include binding moiety-modified cytosines and binding moiety-lacking cytosines. The guanines may include binding moiety-modified guanines and binding moiety-lacking guanines. The cytosines may include binding moiety-modified cytosines and binding moiety-lacking cytosines, and the guanines may include binding moiety-modified guanines and binding moiety-lacking guanines.

The methods may include combining the adenines, thymines, cytosines and guanines in a buffer solution. The composition may include from 1 to 20 percent binding moiety-modified cytosines with the remainder of the cytosines lacking the binding moiety. The composition may include from 2.5 to 10 percent binding moiety-modified cytosines with the remainder of the cytosines lacking the binding moiety. The composition may include from 1 to 20 percent binding moiety-modified guanines with the remainder of the guanines lacking the binding moiety. The composition may include from 2.5 to 10 percent binding moiety-modified guanines with the remainder of the guanines lacking the binding moiety. The composition may include from 1 to 20 percent binding moiety-modified cytosines and guanines with the remainder of the cytosines and guanines lacking the binding moiety. The composition may include from 2.5 to 10 percent binding moiety-modified cytosines and guanines with the remainder of the cytosines and guanines lacking the binding moiety.

The disclosure provides compositions including adenines, thymines, cytosines and guanines wherein the cytosines, guanines, or both cytosines and guanines are included in a mixture of binding moiety-modified nucleotides and binding moiety-lacking nucleotides. The composition may lack or substantially lack binding moiety-modified adenines and lacks binding moiety-modified guanines. The composition may be provided in a buffer solution. The binding moiety-modified nucleotides may include binding moiety-modified cytosines. The binding moiety-modified nucleotides may include binding moiety-modified guanines. The mixture of binding moiety-modified nucleotides and nucleotides lacking the binding moiety may, in certain embodiments, range from 1 to 20 percent binding moiety-modified nucleotides with the remainder of the nucleotides lacking the binding moiety. The mixture of binding moiety-modified nucleotides and nucleotides lacking the binding moiety may, in certain embodiments, range from 2.5 to 10 percent binding moiety-modified nucleotides with the remainder of the nucleotides lacking the binding moiety. The binding moiety-modified nucleotides may include biotin-modified nucleotides.

The invention provides kits. The kits may include any of the compositions of the invention. The kits may, in certain embodiments, include instructions for using the composition. In various embodiments, the kits may include reagents for isolating nucleic acids. In various embodiments, the kits may include a substrate for capturing nucleic acids. In various embodiments, the kits may include reagents for eluting nucleic acids from a substrate. In various embodiments, the kits may include reagents for converting unmethylated cytosines of nucleic acid fragments to uracils. The reagents for converting unmethylated cytosines of nucleic acid fragments to uracils may, for example, include reagents for deaminating the unmethylated cytosines. The reagents for converting unmethylated cytosines of nucleic acid fragments to uracils may, for example, include reagents for converting by enzymatic conversion.

### 5. BRIEF DESCRIPTION OF DRAWINGS

FIG. 1 is a table providing examples of theoretical recovery.

FIG. 2 illustrates a method of enriching for hypermethylated fragments.

FIG. 3 illustrates an embodiment of the disclosure using biotinylated guanines as the binding moiety-modified nucleotide.

FIG. 4 illustrates an embodiment of the disclosure using biotinylated cytosines as the binding moiety-modified nucleotide.

FIG. 5 illustrates additional library preparation steps for sequencing analysis.

FIG. 6 is a schematic diagram illustrating a process of integrating biotin-dNTP labeling and streptavidin enrichment of hypermethylated fragments into a library preparation protocol.

FIG. 7 is a plot showing the expected fold enrichment based on simulations involving various biotin-dGTP percentages.

FIG. 8 is a plot and a table showing cancer classification performance for only hypermethylated targets vs all Compass (baseline) targets.

FIG. 9 is a plot and a table showing cancer signal origin (CSO) classification performance for only hypermethylated targets vs all Compass (baseline) targets.

FIG. 10A is a plot showing the Fragment Analyzer profiles for the libraries prepared using different dNTP mixes.

FIG. 10B is a table showing yields for the libraries prepared using the different dNTP mixes.

FIG. 11 is a plot showing Fragment Analyzer library profile comparisons for V2 GMS control and biotin enriched libraries prepared using the conditions shown in Table 7.

FIG. 12 is a panel of plots showing library profile comparisons for the biotin enriched libraries prepared using 10, 14, and 17 PCR cycles by percent biotin utilized.

FIG. 13 is a plot showing target enriched library profiles for the V2 SOP and biotin enriched libraries.

FIG. 14 is a plot showing a comparison of the mean fragment length by percent biotin-dGTP and biotin-dGTP vendor source for the biotin enriched and V2 SOP control libraries prepared using conditions shown in Table 7.

FIG. 15 is a plot showing the sequencing fragment distributions in the libraries prepared using different biotin-dGTP percentages and vendor sources.

FIG. 16 is a panel of plots showing the mean linear filtered abnormal coverage by target region for total (coverage), hypermethylated (hyper), and hypomethylated (hypo) targets, respectively, for the biotin enriched and V2 SOP control libraries prepared using conditions shown in Table 7.

FIG. 17 is a plot showing a mean abnormal fraction comparison at 75 million subsampled reads for the biotin enriched and V2 SOP control libraries prepared using conditions shown in Table 7.

FIG. 18 is a plot showing an on-target raw fraction comparison between the V2 SOP and biotin enriched libraries prepared using conditions shown in Table 7.

FIG. 19 is a panel of plots showing a comparison of sequencing fragment counts for on-target rates for sequencing data from libraries prepared using the automated V2 GMS target enrichment process and a manual target enrichment process.

FIG. 20 is a pair of plots showing a comparison of CpG enrichment in simulated data and WGBS data, respectively, from biotin enriched libraries relative to V2 SOP libraries.

FIG. 21 is a plot showing abnormal hypermethylation coverage by sequencing depth for the biotin enriched and V2 control libraries.

FIG. 22 is a plot showing the NGS Fragment Analyzer library profile comparison for V2 SOP, Biotin-Enriched_RSB, Biotin-Enriched_HEB, and Biotin-Enriched_original experimental conditions shown in Table 14.

FIG. 23 is a plot showing Biotin-Enriched_HEB library profiles by percentage of biotin-dGTP used in the library preparation protocol.

FIG. 24 is a plot showing the library fragment size distributions for libraries prepared using the 1×B+W buffer (Biotin-Enriched_PCR) and the HEB buffer (Biotin-Enriched_HEB standard PCR) conditions using 10% biotin-dGTP.

FIG. 25 is a plot showing the Fragment Analyzer traces for the library profile comparisons across all biotin-dGTP labeling and V2 control condition described in Table 18.

FIG. 26 is a plot showing the comparison of on-target rates for the libraries in the biotin labeling optimization experiment described in Table 18.

FIG. 27 is a plot showing the on-target rates for the different libraries in the biotin labeling optimization experiment with the V2 control outlier point removed.

FIG. 28A is a plot showing the abnormal coverage of hypermethylated fragments in biotin enriched and V2 control libraries described in Table 18.

FIG. 28B is a plot showing the abnormal coverage of hypomethylated fragments in biotin enriched and V2 control libraries described in Table 18.

FIG. 29A is a plot showing the total coverage of hypermethylated fragments (total_coverage_hyper_cpg_means) in the biotin enriched and V2 control libraries described in Table 18.

FIG. 29B is a plot showing the total coverage of hypomethylated fragments in the biotin enriched and V2 control libraries.

FIG. 30 is a plot showing the abnormal fraction CpG coverage for biotin enriched and V2 control libraries described in Table 18.

FIG. 31 is a plot showing a comparison of sequencing fragment lengths in biotin enriched and V2 control libraries prepared using different percentages of biotin-dGTP described in Table 18.

FIG. 32 is a plot showing the sequencing fragment distributions in biotin enriched and V2 control libraries prepared using different percentages of biotin-dGTP described in Table 18.

FIG. 33 is a plot showing the abnormal coverage of hypermethylated fragments in biotin enriched and V2 control libraries at lower sequencing depths.

FIG. 34 illustrates a schematic diagram of experimental conditions and workflow for the target hybridization enrichment study.

FIG. 35A is a panel of plots showing the Fragment Analyzer profiles for the PC2-V2, Input B-V2, PC2-biotin enriched (PC2-Biotin-Enriched), and Input B-biotin enriched (Input B-Biotin-Enriched) libraries in the hybridization enrichment study.

FIG. 35B is pair of plots showing the total yields by library preparation protocol for the Input B and PC2 libraries.

FIG. 36 is a pair of plots showing fragment counts by sequencing depth for the Input B biotin enriched and V2 control libraries, and the PC2 biotin enriched and V2 control libraries.

FIG. 37 is a plot showing the bisulfite conversion ratio by sequencing depth for the biotin enriched and V2 control Input B and PC2 libraries.

FIG. 38 is a plot showing sequencing fragment length distributions in the biotin enriched and V2 control libraries.

FIG. 39 is a pair of plots showing the on-target rate by depth comparison for the biotin enriched and V2 control libraries.

FIG. 40 is a pair of plots showing the abnormal coverage by depth for hypermethylated fragments in the biotin enriched and V2 control libraries.

FIG. 41 is a pair of plots showing the total coverage by depth for hypermethylated fragments for the biotin enriched and V2 control libraries.

FIG. 42 is a pair of plots showing abnormal fraction coverage for the biotin enriched and V2 control libraries.

### 6. DETAILED DESCRIPTION

### 6.1. Terminology

As used herein the following terms have the meanings given:
“Abnormal fraction coverage” or “abnormal fraction” means the percentage (represented between 0-1) of sequenced fragments with a methylation pattern with abnormal methylation patterns (i.e., unlikely to be observed in healthy patients, and more common in cancer).
“Abnormal target coverage” or “abnormal coverage” means the coverage depth of a region when considering only abnormal fragments after filtering out normal fragments.
“Amplify” or “amplification” means copying a strand of DNA to produce a complementary strand. Amplification may be thermally mediated or may be isothermal. Amplification may, for example, be accomplished by using polymerase to copy a target strand.
“Binding moiety” means a moiety modifying a nucleotide (or a precursor to or derivative of a nucleotide) that exhibits a binding affinity for another molecule or substance and permits the nucleotide (or a precursor to or derivative of a nucleotide) to retain its ability to be incorporated into a nucleic acid strand, in some versions by a polymerase reaction. The binding moiety facilitates capture of a nucleic acid into which the binding moiety-modified nucleotide has been incorporated. Examples of binding moieties include biotin, biotin derivatives, biotin binding protein, digoxygenin, desthiobiotin, and azides for click chemistry.
“Binding moiety-modified nucleotide” means a nucleotide (or a precursor to or derivative of a nucleotide) modified with a binding moiety. Examples of binding moiety-modified nucleotides are binding moiety-modified dCTP and dGTP, such as biotin-modified dCTP (biotin-dCTP) and dGTP (biotin-dGTP).
“Biotin” means biotin or any biotin derivative, including without limitation, substituted and unsubstituted biotin, and analogs and derivatives thereof, as well as substituted and unsubstituted derivatives of caproylamidobiotin, biocytin, desthiobiotin, desthiobiocytin, iminobiotin, and biotin sulfone.
“Biotin-binding protein” means any protein that binds selectively and preferably with high affinity to biotin, including without limitation, substituted or unsubstituted avidin, and analogs and derivatives thereof, as well as substituted and unsubstituted derivatives of streptavidin, ferritin avidin, nitroavidin, nitrostreptavidin, and Neutravidin™ avidin (a de-glycosylated modified avidin having an isoelectric point near neutral).
“Bisulfite conversion” (BSC) means converting cytosine to uracil while leaving 5-methylcytosine or hydroxymethylated cytosine intact. Bisulfite conversion is a technique that is used to study DNA methylation in a sample comprising methylated DNA.
“Bodily fluid” means any bodily fluid containing DNA, including without limitation, whole blood, circulating blood, a blood fraction, serum, or plasma, aqueous humor, ascites, bile, cerebral spinal fluid, chyle, gastric juices, intestinal juices, lymphatic fluid, pancreatic juices, pericardial fluid, peritoneal fluid, pleural fluid, saliva, spinal fluid, sputum, stool or other intestinal waste fluids, sweat, tears, and/or urine.
“cfNA” means extracellular nucleic acids, and “cfDNA” means extracellular DNA, found in a bodily fluid.
“Copying in” or “copy” with respect to a binding moiety-modified nucleotide means introducing the binding moiety-modified nucleotide into a complementary strand via an amplification reaction.
“CpG site” means a region of a DNA molecule where a cytosine nucleotide is followed by a guanine nucleotide in the linear sequence of bases along its 5′ to 3′ direction. “CpG” is a shorthand for 5′-C- phosphate-G-3′, that is, cytosine and guanine separated by only one phosphate group. Cytosines in CpG dinucleotides can be methylated to form 5-methylcytosine.
“Hypermethylated” refers to a methylation status of a DNA fragment containing multiple CpG sites (e.g., at least 2, 3, 4, 5, 6, 7, 8, 9, 10, etc.) where a high percentage of the CpG sites (e.g., 50% or more, 60% or more, 70% or more, 80% or more, 85% or more, 90% or more, or 95% or more, or any other percentage within the range of 50%-100%) are methylated. “Hypermethylated” refers to a nucleic acid fragment having a threshold number of X or more methylated or hydroxymethylated cytosines. In various embodiments, X may be 2 or more, 3 or more, 4 or more, 5 or more, 6 or more, or more. In an embodiment, X is 3, such that hypermethylated fragments are enriched for 3 or more. In another embodiment, X is 4, such that hypermethylated fragments are enriched for 4 or more. In another embodiment, X is 5, such that hypermethylated fragments are enriched for 5 or more.
“Input sample” refers to a processed sample of fragmented DNA. The term “input sample” is used to distinguish from a “sample” which refers to a biological sample obtained from a subject. A sample from a biological subject is processed to prepare an input sample, e.g., by purifying cfDNA from the sample. Nevertheless, it should be noted that in some embodiments, the input sample and the sample may be the same, i.e., the method may be used with a “dirty sample” or an “unpurified sample.”
“Methylated cytosine” includes, unless otherwise indicated, methylated cytosines and/or hydroxymethylated cytosines.
“On-target rate” means the percentage of sequencing data/reads which maps to a region of interest.
“Sample-specific barcode” means nucleic acid segments added to the target nucleic acids from specific sample sources, such as different individuals, tissues, cells, experiments, replicates, or other sources. The sample-specific barcodes permit pooling samples or input samples from multiple sources and sequencing them together. Data from each sample or input sample can later be identified based on the sequences of the sample-specific barcodes.
“Sequencing depth” means the number of times that a given nucleotide has been read in an experiment.
“Target disease” means a disease, condition or target for which an assay or test is being performed, e.g., a target disease may be cancer generally, a specific class of cancers, a specific cancer type, a specific cancer stage, a pre-cancer condition (e.g., nonalcoholic steatohepatitis, nonalcoholic fatty liver disease, fatty liver, cirrhotic liver), combinations of the foregoing, or any other disease or condition or combination of diseases of conditions for which a methylation analysis may produce informative information.
“Total coverage” means the coverage depth of all fragments across a region of interest.
“UMI” means a unique molecular identifier or unique sequence tag. UMIs can be used to identify unique nucleic acid sequences from a nucleic acid sample, such as a fragmented DNA sample, such as a cfDNA sample. UMIs may be provided in numbers sufficient to ensure that each molecule with one or more UMIs will be identifiable. In some cases, a single UMI per molecule will suffice to enable identification of individual molecules. In other cases, 2 or more UMIs per molecule are combined to facilitate identification of individual molecules. In some cases, UMIs are analyzed in sense and antisense directions. In one embodiment, the UMI is or includes a short oligonucleotide sequence having a length of from 2 nt to 100 nt, from 2 nt to 60 nt, from 2 nt to 40 nt, or from 2 nt to 20 nt. In another embodiment, the UMI tag may comprise a short oligonucleotide sequence greater than 5, 6, 7, 8, 9, 10, 11, 12, 13, 14, 15, 16, 17, or 18 nucleotides (nt) in length.

The invention is not limited to particular embodiments described which one of skill in the art will recognize, may vary within the scope of the invention. The terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

As used herein and in the appended claims, the singular forms “a”, “an”, and “the” include plural referents unless the context clearly dictates otherwise. Thus, for example, reference to “a nucleic acid” includes a plurality of such nucleic acids and reference to “the mixture” includes reference to one or more mixtures and equivalents thereof known to those skilled in the art, and so forth.

The claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publications may set out definitions of a term that conflict with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### 6.2. Enrichment for Methylated DNA Fragments

The disclosure provides a method of enriching an input sample of nucleic acid fragments. In some cases, each fragment in the input sample may have zero, one or more methylated cytosines. The method enables the enrichment of the input sample to preferentially retain fragments exceeding a predetermined methylated cytosine count, while eliminating a portion of the fragments having a methylated cytosine count not exceeding the threshold. For example, the method enables the enrichment of the input sample to preferentially retain fragments exceeding a methylated cytosine count selected from 1, 2, 3, 4, 5, 6 or greater, while eliminating a portion of the fragments having a methylated cytosine count not exceeding the selected methylated cytosine count.

### 6.3. Operation

The methods make use of the incorporation of binding moiety-modified nucleotides into copies of input sample nucleic acids. The binding moiety-modified nucleotides may be incorporated into (“copied into”) a copy of the target strand and used capture the target strand. The binding moiety-modified nucleotides are selectively incorporated into the copies at the positions of methylated cytosines or at positions complementary to methylated cytosines.

Incorporation of binding moiety-modified nucleotides is selective for methylated cytosines. In one embodiment, this selectivity is achieved by chemically altering or blocking the unmethylated cytosines. In one example, bisulfite treatment can be used to convert unmethylated cytosines to uracils, leaving the methylated cytosines available to guide the introduction of binding moiety-modified nucleotides via polymerase extension.

Bisulfite conversion, for example, uses sodium bisulfite to convert cytosine into uracil while keeping 5-methylcytosine (5-mC) unchanged in DNA. Bisulfite conversion may be used to prepare DNA for input in a methylation sequencing library preparation protocol.

Binding moiety-modified nucleotides may be incorporated into (“copied into”) a complementary strand during a strand copying step, such as a primer extension reaction mediated by polymerase. For example, a binding moiety-modified guanine may be introduced during a strand copying step opposite the methylated cytosine. As another example, a binding moiety-modified cytosine may be introduced by copying a methylated strand to produce a new strand in which methylated cytosines are copied as guanines and then copying the new strand to further to convert the guanines to binding moiety-modified cytosines.

Enrichment of the sample for fragments with higher methylated cytosine count is facilitated by conducting the amplification reaction to replace the methylated cytosine with a replacement nucleotide. To enrich for fragments with higher methylated cytosine count, the replacement nucleotide is supplied as a mixture of binding moiety-modified nucleotide and unmodified nucleotide.

The inventors have found that the recovery of fragments may be estimated based on the following formula:

1-(1−%B)^{#M}

where % B is the percent of binding moiety-modified nucleotide used in the amplification step, e.g., 10% refers to 10% binding moiety-modified nucleotide, 20% refers to 20% binding moiety-modified nucleotide, and so on; and #M is the number of methylated cytosines in the fragment.

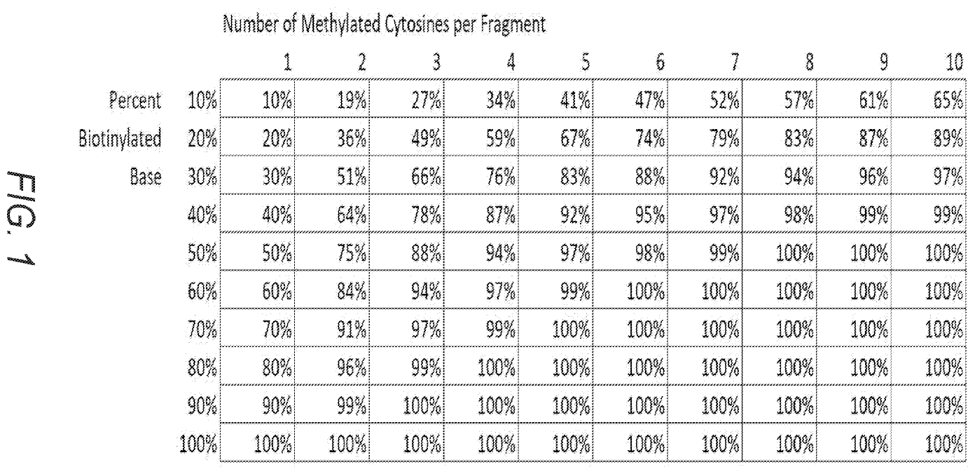

FIG. 1 is a table 100 that provides examples of theoretical recovery. The numbers 1, 2, 3, etc. along the top refer to fragments with the indicated number of methylated cytosines per fragment, e.g., 1 refers to DNA fragments with 1 methylated cytosine, 2 refers to DNA fragments with 2 methylated cytosines, and so on. The percentages along the left side indicate the proportion of binding moiety-modified nucleotide used in the amplification step, e.g., 10% refers to 10% binding moiety-modified, 20% refers to 20% binding moiety-modified, and so on. The percentages are given in 10% increments as a convenient illustration only; it will be appreciated that any percentages may be used. The numbers in the body of the table indicate the theoretically expected percentage of fragments in a mixture, i.e., the quantity fragments having the indicated number of methylated cytosines that will theoretically be captured by the method at the given percent binding moiety-modified nucleotide. It will be appreciated that actual recovery may vary based on reaction conditions and other factors.

For example, referring to FIG. 1, the incorporation of 10% binding moiety-modified nucleotides is expected to result in capture of fragments with one methylated cytosine at 10%, capture of fragments with two methylated cytosines at 19%, and so on.

Because samples usually include more fragments with lower numbers of methylated cytosines than fragments with higher numbers of methylated cytosines, this technique can eliminate a substantial number of molecules from downstream processing, thereby significantly increasing efficiency of the subsequent steps, including the sequencing step.

It will be appreciated that where recovery is lower, total recovery of targets may be increased by amplification of the library prior to the enrichment step. Thus, for example, 1, 2, 3, 4, 5, 6, 7, 8, 9, 10 or more linear amplification rounds may be performed.

The method may be used to enrich for fragments having a threshold number of X or more methylated or hydroxymethylated cytosines. In various embodiments, X may be 2, 3, 4, 5, 6, or more. In one embodiment, X is 3, such that hypermethylated fragments are enriched for 3 or more. In another embodiment, X is 4, such that hypermethylated fragments are enriched for 4 or more. In another embodiment, X is 5, such that hypermethylated fragments are enriched for 5 or more. The enriched sample produced by the method may be subjected to additional library preparation steps and sequence analysis, e.g., by sequencing or microarray.

### 6.4. Method of Enriching for Hypermethylated Fragments

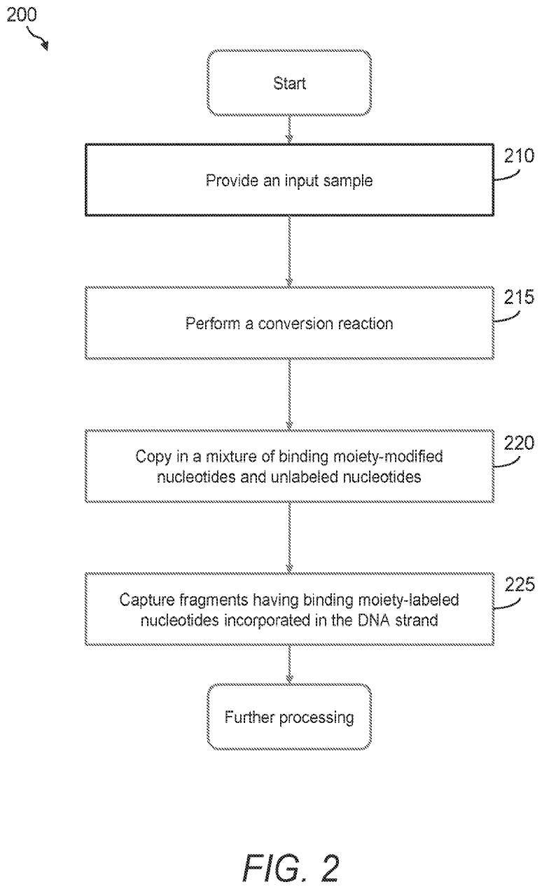

FIG. 2 is a flow diagram illustrating a method 200 of enriching for hypermethylated fragments, which includes but is not limited to, the following steps:

### 6.4.1. Input Sample

At a step 210, an input sample is provided. The input sample includes fragmented DNA. The fragmented DNA may, for example, be fragmented genomic DNA or cfDNA. The input sample may be any subset of a genome, including a whole genome or even multiple genomes.

The sample source may be any source of DNA. For example, the sample source may be a biological organism or an environmental sample. Where the sample source is a biological organism, the sample source may be tissues, cells, fluids, or other substances. The sample may be fresh or may be preserved by various preservation techniques. In some instances, the subject is a human or other animal. Samples or input samples may in some cases be pooled from multiple sources and/or multiple subjects. Sample barcodes or indexes coupled to fragments may be used to distinguish pooled samples from one another.

In some cases, the sample is from a subject known to have or suspected of having a target disease. In some cases, the sample is from a subject not known to have or suspected of having a target disease (e.g., a control subject in a study or a subject undergoing screening for a disease).

In some cases, the sample is from a subject known to have or suspected of having a cancer. In some cases, the sample is from a subject not known to have or suspected of having cancer (e.g., a control subject in a study or a subject undergoing screening for cancer).

In some embodiments, the sample is a tumor sample or a suspected tumor sample. In some embodiments, the sample is a tissue sample that may be a cancer tissue. In some embodiments, the sample is a tissue sample that may be a stage I, II, III, or IV cancer.

In some embodiments, the sample is a bodily fluid or other extracellular bodily substance. In some embodiments, the bodily fluid or other extracellular bodily substance is selected from the group consisting of whole blood, a blood fraction, serum, and plasma. In some embodiments, the bodily fluid or other extracellular bodily substance is selected from aqueous humor, ascites, bile, cerebral spinal fluid, chyle, gastric juices, intestinal juices, lymphatic fluid, pancreatic juices, pericardial fluid, peritoneal fluid, pleural fluid, saliva, spinal fluid, sputum, stool or other intestinal waste fluids, sweat, tears, and/or urine.

In some embodiments, the input sample includes cfNA or cfDNA obtained from a bodily fluid or other bodily substance. In some cases, the cfNA or cfDNA originate from healthy cells. In some cases, the cfNA or cfDNA originate from diseased cells, such as cancer cells.

6.4.1.1. Purification of cfNA

In some cases, DNA is extracted or purified from a sample to provide the input sample. (Note that in other cases, a raw sample may be used as an input sample.)

Where the sample is a bodily fluid or substance and the input sample is a cfNA sample, a variety of methods can be used to extract and purify cfNA from the sample.

Kits and methods are commercially available for purifying DNA from tissues and/or cells. Examples include Genomic DNA Isolation Kit (LifeSpan BioSciences, Inc., Seattle, Washington); Genomic DNA Isolation Kit (MyBioSource, Inc., San Diego, California); Genomic DNA Isolation Kit (Biorbyt Ltd., Cambridge, United Kingdom). The product literature of these kits is incorporated herein by reference.

Kits and methods are commercially available for purifying cfNA from blood. Examples include QIAamp Circulating Nucleic Acid Kit (QIAGEN, N.V., Hilden, Germany); PME free-circulating DNA Extraction Kit (Analytik Jena AG, Jana, Germany); Maxwell RSC ccfDNA Plasma Kit (Promega Corporation, Madison, Wisconsin); EpiQuick Circulating Cell-Free DNA Isolation Kit (Epigentek Group Inc., Farmingdale, New York); NEXTprep-Mag cfDNA Isolation Kit (PerkinElmer, Waltham, MA). The product literature of these kits is incorporated herein by reference.

Kits and methods are commercially available for purifying cfNA from urine. Examples include QIAamp DNA Micro Kit (QIAGEN, N.V., Hilden, Germany); QIAamp Viral RNA Mini Kit (QIAGEN, N.V., Hilden, Germany); i-genomic Urine DNA Extraction Mini Kit (iNtRON Biotechnology, Inc, South Korea); Quick-DNA Urine Kit (Zymo Research Corp., Irvine, California); Norgen RNA/DNA/Protein Purification Plus Kit (Norgen Biotek Corp, Thorold, Ontario, Canada); and Abcam DNA Isolation Kit—Urine (Abcam Plc., Cambridge, United Kingdom). The product literature of these kits is incorporated herein by reference.

Other kits and methods are available for isolating DNA from other bodily fluids and substances.

### 6.4.1.2. Fragmenting DNA

In some cases, it may be necessary to fragment DNA from a sample to produce an input sample. Various known methods of fragmenting DNA may be used, including for example, acoustic shearing, sonication, hydrodynamic shearing, restriction endonucleases (such as DNase I), or transposases.

### 6.4.1.3. Target Enrichment

In some cases, fragmented DNA is enriched for targets of interest. For example, in some embodiments the input sample itself may be enriched for targets prior to initiating the process illustrated in FIG. 2. In some embodiments, target enrichment may occur prior to performing a conversion reaction (i.e., a step 215). In some embodiments, target enrichment may occur after performing the conversion reaction (i.e., step 215) and prior to performing a step of copying in a mixture of binding moiety-modified nucleotides and unmodified nucleotides (i.e., a step 220). It is also possible to perform target enrichment following a step for capture and optionally, elution of strands having binding moiety-modified nucleotides (i.e., a step 225).

For example, DNA may be enriched for targets or fragments from genomic regions predictive, or potentially predictive, of a disease state or condition, such as a cancer, cancer type, cancer tissue of origin, and/or cancer stage.

DNA fragments provided in an input sample are in various instances targets that have a possibility of being hypermethylated. Various disclosed targets have a threshold number of X or more CpG sites. In various embodiments, X may be 2, 3, 4, 5, 6, or more. In one embodiment, X is 3, such that hypermethylated fragments are enriched for 3 or more. In another embodiment, X is 4, such that hypermethylated fragments are enriched for 4 or more. In another embodiment, X is 5, such that hypermethylated fragments are enriched for 5 or more.

DNA targets may include those which are known to be hypermethylated in cancer samples relative to non-cancer samples and/or those which are hypermethylated in non-cancer samples relative to cancer samples. DNA targets may include fragments for which hypermethylation is associated with cancer samples relative to non-cancer samples and/or those for which hypermethylation is associated with non-cancer samples relative to cancer samples.

DNA targets may include those for which hypermethylation is associated with origination in a specific organ or specific organs relative to other organs. DNA targets may include cfDNA fragments for which hypermethylation is associated with origination in a specific organ or specific organs relative to other organs. DNA targets may include cfDNA fragments for which hypermethylation is associated with excluding origination in a specific organ or specific organs relative to other organs. DNA targets may include those which are hypermethylated in certain organs relative to other organs.

DNA targets may include those for which hypermethylation is associated with origination in a specific tissue or specific tissues relative to other tissues. DNA targets may include cfDNA fragments for which hypermethylation is associated with origination in a specific tissue or specific tissues relative to other tissues. DNA targets may include cfDNA fragments for which hypermethylation is associated with excluding origination in a specific tissue or specific tissues relative to other tissues. DNA targets may include those which are hypermethylated in certain tissues relative to other tissues.

DNA targets may include those for which hypermethylation is associated with origination in a specific cell-type or specific cell-types relative to other cell-types. DNA targets may include cfDNA fragments for which hypermethylation is associated with origination in a specific cell-type or specific cell-types relative to other cell-types. DNA targets may include cfDNA fragments for which hypermethylation is associated with excluding origination in a specific cell-type or specific cell-types relative to other cell-types. DNA targets may include those which are hypermethylated in certain cell-types relative to other cell-types.

A bait set may be provided for hybridization capture of targets. The bait set may comprise a plurality of different oligonucleotide-containing probes. The bait set may comprise at least 10, 50, 100, 200, 300, 400, 500, 1,000, 2,000, 2,500, 5,000, 6,000, 7,500, 10,000, 15,000, 20,000, 25,000, 50,000 or 100,000 or more different oligonucleotide-containing probes.

Typically, each of the oligonucleotide-containing probes of the bait set comprises a sequence of at least 30 bases in length that is complementary to a pre- or post-bisulfite conversion target.

Typically target enrichment is accomplished by capturing genomic regions of interest by hybridization to target-specific DNA or RNA probes specific to the target regions of interest. The hybridization between DNA libraries and baits may, in some embodiments, be carried out in solution or on a solid support. In “solid-phase,” DNA probes are bound to a solid support, such as a bead or glass microarray slide. In some cases, the hybridization capture step can be repeated in 2 or more rounds to enhance the quantity of targets captured. In other cases, only a single round of the hybridization capture step is used.

In “solution-capture,” free DNA or RNA probes are typically biotinylated allowing them to isolate the targeted fragment-probe duplexes using magnetic biotin-binding protein-coated beads, such as streptavidin-coated beads. The biotin moiety can be added to the 5′-end of the probes. Captured targets may be isolated by magnetic pulldown, e.g., using magnetic biotin-binding protein-coated beads, such as streptavidin-coated beads. For a solution-based hybridization method that includes the use of biotinylated oligonucleotides and streptavidin-coated magnetic beads, see, e.g., Duncavage et al., J Mol Diagn. 13(3): 325-333 (2011); and Newman et al., Nat Med. 20(5): 548-554 (2014), the entire disclosure of which are incorporated herein by reference.

In some embodiments, a sample can be enriched for targets of interest (e.g., cancer-associated genes) using other methods known in the art, such as hybrid capture. See, e.g., Lapidus, U.S. Pat. No. 7,666,593, issued on Feb. 23, 2010, the entire disclosure of which is incorporated herein by reference.

In some embodiments, a sample can be enriched for targets of interest, and the targets of interest may include targets that are potentially hypermethylated. In some embodiments, a sample can be enriched by a single round of hybridization capture for targets of interest that are potentially hypermethylated. In some embodiments, a sample can be enriched by two rounds of hybridization capture for targets of interest that are potentially hypermethylated. In some embodiments, a sample can be enriched by more than two rounds of hybridization capture for targets of interest that are potentially hypermethylated.

Non-specific unbound molecules may be washed away, and the enriched DNA subjected to subsequent steps in the process.

In the process illustrated here, enrichment occurs prior to the conversion step (i.e., step 215). However, it should be noted that an enrichment step can follow the bisulfite conversion step, by using probes designed to select for post-conversion fragments.

It should also be noted that in some cases an amplification step may be performed prior to the conversion step (i.e., step 215) using DNA methyltransferase to catalyze methyl group transfer to the new strands.

### 6.4.2. Conversion of Cytosines

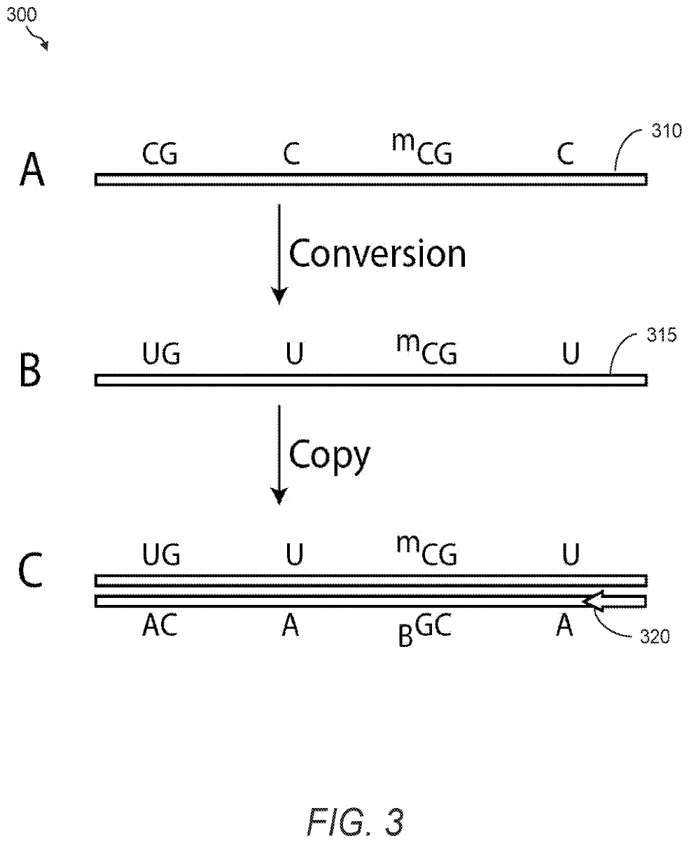

FIG. 3 illustrates an embodiment of the disclosure using biotinylated guanines as the binding moiety-modified nucleotide.

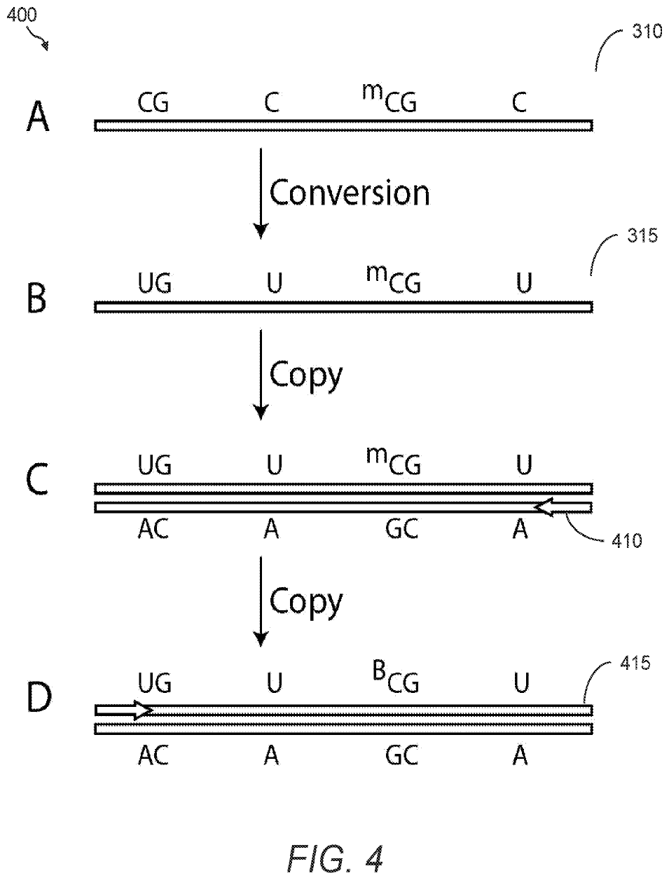

FIG. 4 illustrates an embodiment of the disclosure using biotinylated cytosines as the binding moiety-modified nucleotide.

As illustrated in FIG. 2, step 215, and FIG. 3 and FIG. 4, step A, a conversion reaction is performed in which cytosines (C) in starting strand 310 are selectively converted into uracils (U) in converted strand 315.

A variety of kits are commercially available for this purpose. Examples include EPIMARK Bisulfite Conversion Kit (New England Biolabs Ltd., Ipswich, Massachusetts); ACTIVEMOTIF Bisulfite Conversion Kit (Active Motif, Inc., Carlsbad, California); EPITECT Bisulfite Kits (QIAGEN Ltd., Hilden, Germany); EZ DNA Methylation-Lightning Kit (Zymo Research Corp., Irvine, California); NEBNext® Enzymatic Methyl-seq (EM-seq™) (New England Biolabs, Inc., Ipswich, Massachusetts). The product literature of these kits is incorporated herein by reference.

### 6.4.2.1. Chemical Conversion

In one embodiment, the DNA fragments are denatured and treated with a bisulfite. The denaturation and bisulfite treatment steps can be in a single reaction or can be conducted sequentially. Bisulfite treatment modifies unmethylated cytosines with a sulfite. After conversion, the DNA may be deaminated to convert to uracil. For example, the DNA may be desalted and incubated at alkaline pH resulting in deamination and conversion to uracil.

In one example, the DNA fragments may be denatured with NaOH at a final concentration of 0.3 N and treated with sodium bisulfite or sodium metabisulfite at a final concentration of 2 M (pH between 5 and 6) at 55° C. for 4-16 hours. After conversion, the DNA is desalted followed by desulfonation by incubating the DNA at alkaline pH at room temperature.

### 6.4.2.2. Enzymatic Conversion

In another embodiment, the conversion of unmethylated cytosines to uracils makes use of enzymatic techniques. For example, certain cytosine deaminases are known for deaminating cytosine bases to uracil in single-stranded DNA.

In one example, the cytosine deaminase is APOBEC. APOBEC also deaminates 5mC and 5hmC, so in order to detect 5mC and 5hmC, these methods use techniques to block deamination of 5mC and/or 5hmC. For example, using EM-seq™ (New England Biolabs, Ipswich, Massachusetts), TET2 and an oxidation enhancer can be used to modify 5mC and 5hmC to forms that are not substrates for APOBEC. The TET2 enzyme converts 5mC to 5caC, and the oxidation enhancer converts 5hmC to 5ghmC. The NEBNext® Enzymatic Methyl-seq (EM-seq™) product literature is incorporated herein by reference.

In another embodiment, APOBEC-coupled epigenetic sequencing (ACE-seq) relies on enzymatic conversion to detect 5hmC. With this method, T4-BGT glucosylates 5hmC to 5ghmC and protects it from deamination by APOBEC3A. Cytosine and 5mC are deaminated by APOBEC3A and sequenced as thymine.

In another embodiment, oxidative bisulfite sequencing (oxBS) is used to distinguish between 5mC and 5hmC. The oxidation reagent potassium perruthenate converts 5hmC to 5-formylcytosine (5fC) and subsequent sodium bisulfite treatment deaminates 5fC to uracil. 5mC remains unchanged and can therefore be identified using this method.

In another embodiment, fragmented DNA is treated with T4-BGT which protects 5hmC by glucosylation. The enzyme mTET1 is then used to oxidize 5mC to 5hmC, and T4-BGT labels the newly formed 5hmC using a modified glucose moiety (6-N3-glucose).

### 6.4.3. Strand Denaturation

In some cases, the strands are denatured prior to conducting the conversion reaction (i.e., step 215). Denaturation may, for example, be accomplished by incubation at elevated temperatures, e.g., 98° C., and/or exposure to a base, such as sodium hydroxide.

### 6.4.3.1. Denaturation on Substrate

As noted above, various steps in the conversion process may be performed while the DNA is captured on a substrate, such as a column matrix on beads. This facilitates washing to remove contaminants, such as dNTPs and salts. In another embodiment, DNA may be captured on a substrate, such as a column matrix on beads, following conversion for washing. In one example, SPRI paramagnetic bead-based chemistry is used for capture and washing. For example, AMPure XP for PCR Purification (Beckman Coulter, Inc., Pasadena, California) may be used.

In some cases, DNA fragments may be eluted before moving to the next step in the process. In some embodiments, the next steps may be performed on-bead or on-surface without eluting the DNA.

### 6.4.4. Copy in a Mixture of Binding Moiety-Modified Nucleotides and Unmodified Nucleotides

In FIG. 2, at a step 220, FIG. 3, step C, and FIG. 4, steps C and D, the converted fragments are copied to add in binding moiety-modified nucleotides, e.g., using a primer extension reaction.

In the conversion step described above (step 215), the unmethylated cytosines are converted to uracils, leaving the methylated cytosines. During the first-round amplification reaction (formation of the first copy), the methylated cytosines pair with guanines. In the second step of the amplification reaction, the guanines pair with cytosines. Thus, the methods may make use of binding moiety-modified guanines or binding moiety-modified cytosines copied into the strand to capture strands with methylated cytosines.

In the embodiment illustrated in FIG. 3 step C, a mixture of binding moiety-modified guanines is used in the amplification or primer extension reaction, to produce from converted strand 315 a copy 320 in which a proportion of the guanines are binding moiety-modified guanines (illustrated here as B^{GC}). For example, the binding moiety-modified guanines may be biotinylated guanines.

In the embodiment illustrated in FIG. 4, during the first amplification or primer extension reaction step C, the methylated cytosines in converted strand 315 pair with guanines to produce strand 410. In the second amplification or primer extension reaction step D, a mixture of binding moiety-modified cytosines is used to copy strand 410 and produce copies 415 in which a proportion of the cytosines are binding moiety-modified cytosines (illustrated here as B_{CG}). For example, the binding moiety-modified cytosines may be biotinylated cytosines.

In one embodiment, the proportion of binding moiety-modified nucleotide in the mixture ranges from 1 to 50 percent binding moiety-modified nucleotides with the remainder of the nucleotides lacking the binding moiety. In one embodiment, the proportion of binding moiety-modified nucleotide in the mixture ranges from 1 to 40 percent binding moiety-modified nucleotides with the remainder of the nucleotides lacking the binding moiety. In one embodiment, the proportion of binding moiety-modified nucleotide in the mixture ranges from 1 to 30 percent binding moiety-modified nucleotides with the remainder of the nucleotides lacking the binding moiety. In one embodiment, the proportion of binding moiety-modified nucleotide in the mixture ranges from 1 to 20 percent binding moiety-modified nucleotides with the remainder of the nucleotides lacking the binding moiety. In one embodiment, the proportion of binding moiety-modified nucleotide in the mixture ranges from 2.5 to 10 percent binding moiety-modified nucleotides with the remainder of the nucleotides lacking the binding moiety. In one embodiment, the proportion of binding moiety-modified nucleotide in the mixture is less than 10 percent binding moiety-modified nucleotides with the remainder of the nucleotides lacking the binding moiety.

In these and other embodiments, the binding moiety-modified nucleotide may, for example, be a biotin-modified nucleotide, with the remainder being unmodified nucleotide. In these and other embodiments, the binding moiety-modified nucleotide may, for example, be biotin-modified guanine, with the remainder being unmodified guanine. In these and other embodiments, the binding moiety-modified nucleotide may, for example, be biotin-modified cytosine, with the remainder being unmodified cytosine.

In one embodiment, the proportion of binding moiety-modified nucleotide in the mixture is selected to enrich for fragments with X and greater methylated cytosines, where X=1, 2, 3, 4, 5, 6, 7, 8, 9 or 10. In one embodiment, X is 1. In one embodiment, X is 2. In one embodiment, X is 3. In one embodiment, X is 4. In one embodiment, X is 5. In one embodiment, X is 6. In one embodiment, X is 7. In one embodiment, X is 8. In one embodiment, X is 9. In one embodiment, X is 10.

The proportion of binding moiety-modified nucleotide required to produce the desired capture results will vary depending on the binding chemistry used and other factors known to those of skill in the art. The proportion of binding moiety-modified nucleotide required to produce the desired capture results can be determined experimentally by testing a standard sample across a series of proportions of modified/unmodified nucleotide to produce a curve describing the results for the particular chemistry selected. Alternatively, the curve can be generated by modeling in silico.

The primer extension reaction uses an enzyme that is able to read through uracil residues in the converted ssDNA template strand. For example, Klenow fragment (3′→45′ exo-) DNA polymerase (available from New England Biolabs, Ltd., Ipswich, MA) can be used in the primer extension reaction to form the converted dsDNA construct. Product literature for Klenow fragment (3′→45′ exo-) DNA polymerase is incorporated herein by reference. In another example, Taq or Archaea enzymes modified to accept uracil templates may be used.

Following copying of the uracil-containing strand, the original strand can be degraded, e.g., using USER® Enzyme (New England Biolabs, Corp, Ipswitch, Massachusetts). Product literature for USER® Enzyme is incorporated herein by reference.

### 6.4.4.1. Binding-Moiety Modified Nucleotides

A variety of binding moiety-modified nucleotides are commercially available. For example, biotin-11-dCTP, biotin-14-dCTP, biotin-16-dCTP, biotin-11-dGTP, biotin-14-dGTP, biotin-16-dGTP, are commercially available from various companies, including for example, one or more of the following: Biotium, Inc., Fremont, California; Jena Bioscience GmbH, Jena, Germany; Thermo Fisher Scientific, Waltham, Massachusetts; and Perkin Elmer, Inc., Waltham, Massachusetts.

The invention may also make use of cleavable binding moieties, such as cleavable biotin analogues. For example, incorporation of a biotin with a linker arm containing a disulfide bond allows for a simple dissociation of the DNA fragment, as the disulfide links easily become cleaved with dithiothreitol (DTT).

6.4.5. Capture Fragments having Binding Moiety-Modified Nucleotides

In a step 225, fragments with incorporated binding moiety-modified nucleotides are captured. For example, fragments with binding moiety-modified nucleotides incorporated into the DNA strand are captured using a support, such as a solid support, having affinity for the binding moiety. Capture facilitates washing to remove contaminants, such as unmodified strands, dNTPs and salts. For example, biotin-modified strands can be captured using a biotin-binding protein-coated solid support, such as a streptavidin solid support, such as streptavidin coated beads or wells. In another embodiment, DNA may be captured on a substrate, such as a column matrix or on beads, such as glass or silica beads, such as magnetic glass or silica beads, following conversion (step 215) for washing prior to performing subsequent steps. In one example, SPRI paramagnetic bead-based chemistry is used for capture and washing. For example, AMPure XP for PCR Purification (Beckman Coulter, Inc., Pasadena, California) may be used. The output of the capture step 225 is an enriched sample, i.e, the input sample has been enriched for the desired degree of methylation.

DNA fragments of the enriched sample may be eluted before moving to the next step in the process. In some embodiments, the next steps may be performed on-bead or on-surface without eluting the DNA.

The enriched sample may be analyzed by a variety of DNA analysis techniques, such as PCR assays, capture assays, microarrays, and sequencing.

The composition may thus be enriched for informative fragments. The complexity of the library may thus be reduced relative to the input sample. Enrichment for informative fragments and/or reduction in complexity, may facilitate a reduction in the sequencing depth required for conducting subsequent analyses, such as methylation assays.

### 6.5. Additional Processing Steps for Sequencing Analysis

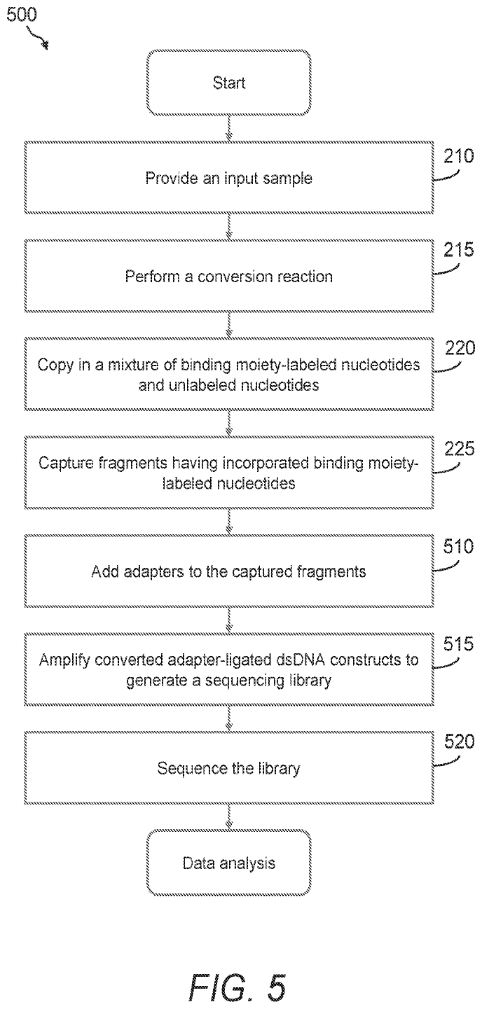

FIG. 5 is a flow diagram of an example of a method 500 of preparing a library for methylation profiling using sequencing. In this example, steps 210 through 225 are as described with reference to FIG. 2, as further illustrated by FIG. 3 and FIG. 4.

### 6.5.1. Adding Adapters

### 6.5.1.1. Adding Adapters at One End, Then the Other End

At a step 510, sequencing adapters are added to the captured fragments. In one embodiment, a first adapter is added to the 3′-OH ends of the converted ssDNA fragments in a first ligation reaction to generate a plurality of converted adapter-ligated ssDNA fragments or constructs. For example, a first adapter is added to the 3′-OH end of a converted ssDNA fragment using a single-stranded DNA (ssDNA) ligase and a reaction buffer that includes polyethylene glycol (PEG). Any ssDNA ligase can be used.

Optionally, in one embodiment, a dephosphorylation/denaturation reaction is performed prior to the adapter ligation step to generate dephosphorylated, converted single-stranded DNA (ssDNA). For example, the ssDNA ligation reaction uses a ssDNA ligase, such as CircLigase II (Epicentre Technologies Corp., Madison, Wisconsin), to ligate a first adapter to the 3′-OH end of a bisulfite-converted ssDNA fragment.

In another embodiment, the ssDNA ligation reaction uses a thermostable RNA ligase, such as Thermostable 5′ AppDNA/RNA ligase (available from New England BioLabs (Ipswich, MA)), to ligate a first adapter to the 3′-OH end of a bisulfite-converted ssDNA fragment.

In another embodiment, the first adapter includes, for example, a 5′-phosphate, a first universal primer sequence (e.g., an SBS primer sequence), and optionally can be blocked at the 3′-end (e.g., 3′-ddNTP) to inhibit adapter-dimer formations.

An adapter purification step (not shown) can be used to digest incomplete synthesized adapters and unblocked adapters prior to use of the adapters in the ligation reaction.

In one embodiment, as noted above, the first ligation reaction is performed in a reaction buffer that includes polyethylene glycol (PEG). The reaction buffer may, for example, include at least 5%, at least 10%, at least 15%, at least 20%, at least 25%, at least 30%, at least 40% polyethylene glycol. In another embodiment, the reaction mixture may include from 5% to 40%, from 10% to 30%, or from 15% to 25% polyethylene glycol. In another embodiment, the reaction buffer comprises 20% polyethylene glycol. The Applicants have found that the inclusion of polyethylene glycol in the reaction mixture results in enhanced ligation of the first adapter to the converted, ssDNA fragments, and thus, results in an improved recovery of sequenceable fragments.

The ssDNA adapters may optionally include one or more UMI sequences. UMIs can be used to reduce amplification bias, which is the asymmetric amplification of different targets due to differences in nucleic acid composition (e.g., high GC content). UMIs can also be used to discriminate between nucleic acid mutations that arise during amplification.

In some cases, the ssDNA adapters specifically omit UMIs, that is, they do not include UMIs, and the associated methods of analysis do not include UMI based analyses, such as UMI based error correction.

The ssDNA adapters may optionally include one or more sample-specific barcode sequences, sometimes referred to as sample indexes. The sample-specific barcode may be selected to distinguish data produced during a sequencing run from specific samples or sets of samples pooled together in a sequencing run from other samples or sets of samples. Data from each sample can later be identified by computer analysis based on the sequences of the sample-specific barcodes.

In another aspect of the invention, the ssDNA adapters utilized in the practice of this invention may include a universal primer and/or one or more sequencing oligonucleotides for use in subsequent cluster generation and/or sequencing (e.g., known P5 and P7 sequences for used in sequencing by synthesis (SBS) (Illumina, San Diego, CA)).

Optionally, a bead-based cleanup protocol may be performed on the adapter ligated ssDNA constructs. In one example, the cleanup protocol is a 1.8× SPRI-cleanup protocol that is performed on the adapter ligated ssDNA using a reaction buffer that includes PEG (e.g., from 15% to 20% PEG).

A second strand DNA may be synthesized in a primer extension reaction to generate double-stranded DNA (dsDNA) constructs. For example, the 3′-end of the ssDNA adapters may be extended using a DNA polymerase, and the ssDNA fragment as a template, to generate a plurality of double-stranded DNA (dsDNA) molecules. For example, a DNA polymerase can be used to synthesize, from the free 3′-ends of the ssDNA adapters, a nucleic acid sequence complementary to the converted ssDNA fragment. Any DNA polymerase can be used. For example, the polymerase used in the practice of the present invention can be Bst 2.0 (New England BioLabs, Ipswich, MA), Dpo4 (Dpo4), T4 DNA polymerase (T4 DNA polymerase), or DNA polymerase I (New England BioLabs, Ipswich, MA).

At this step in the process, an optional, bead-based cleanup protocol may be performed on the adapter ligated dsDNA constructs. In one example, the cleanup protocol is a 1.8× SPRI-cleanup protocol that is performed on the adapter ligated dsDNA using a reaction buffer that includes PEG (e.g., from 15% to 20% PEG).

Continuing step 510, a second ligation reaction may be performed to ligate a second adapter to the 5′-end of the converted dsDNA construct to generate a plurality of dsDNA adapter-fragment constructs. For example, a second adapter may be a double-stranded adapter that includes a universal primer sequence (e.g., an SBS primer sequence), wherein one strand includes a 5′-phosphate and optionally the other strand includes a 3′-block.

### 6.5.1.2. Adding Adapters at Both Ends

Optionally, in another embodiment, dsDNA adapters can be ligated to both ends of the converted dsDNA constructs obtained from step 220 (as further illustrated by FIG. 3 step C and FIG. 4 step D). The ligation reaction can be performed using any suitable ligase enzyme which joins the dsDNA adapters to the dsDNA fragments to form dsDNA adapter-fragment constructs. In one example, the ligation reaction is performed using T4 DNA ligase. In another example, T7 DNA ligase is used for adapter ligation to the modified nucleic acid molecule.

In one embodiment, the ends of dsDNA fragments are first repaired using, for example, T4 DNA polymerase and Klenow polymerase and phosphorylated with a polynucleotide kinase enzyme. A single “A” deoxynucleotide is then added to the 3′ ends of dsDNA fragments using, for example, Taq polymerase enzyme, producing a single base 3′ overhang that is complementary to a 3′ base (e.g., a T) overhang on the dsDNA adapter.

Like the ssDNA adapters described above, the dsDNA adapters may comprise one or more UMI sequences or may specifically exclude UMI sequences.

A bead-based cleanup protocol may be performed on the adapter ligated, converted dsDNA construct. For example, in one embodiment, the cleanup protocol is a 1.8× SPRI-cleanup protocol.

6.5.2. Amplifying Converted Adapter-Ligated dsDNA Constructs to Generate a Sequencing Library

At a step 515, the converted adapter-ligated dsDNA constructs are amplified to generate a sequencing library. For example, as is known in the art, the adapter-fragment dsDNA constructs can be amplified by PCR using a DNA polymerase and a reaction mixture containing primers and a plurality of dNTPs. In one embodiment, sequencing adapters and sample-specific index sequences can be added during the amplification step. For example, PCR amplification using a forward primer that includes a P5 sequence and a reverse primer that includes a P7 sequence and an index sequence is used to add P5, P7, and sample-specific index sequences to the converted dsDNA adapter-ligated constructs. The converted dsDNA library is now ready for sequencing and subsequent analysis to determine, for example, methylation sites and patterns.

### 6.5.3. Sequencing the Amplified Library

At a step 520, sequence reads are generated from the amplified fragments of the sequencing library. The sequencing method may include any known sequencing method, including for example, next generation sequencing (NGS) techniques, including synthesis technology (Illumina), pyrosequencing (454 Life Sciences), ion semiconductor technology (Ion Torrent), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators.

Sequence reads may then be aligned to a reference genome. Alignment permits identification of methylated CpG sites on the cfDNA fragment. Methylation status can be used in an algorithm to characterize disease states, including for example, cancer yes/no, cancer type, and tissue of origin.

### 6.5.4. Analyzing Sequencing Results

In one embodiment, hypermethylated fragments exceeding a methylation threshold are identified and used as input into an algorithm for characterizing disease states, including for example, cancer yes/no, cancer type, and tissue of origin.

For example, in one embodiment, data produced by the methods of the invention may feed into an analytics system as described in U.S. Patent Pub. No. 20190287652, entitled “Anomalous fragment detection and classification,” by Gross et al., the entire disclosure of which is incorporated herein by reference. Thus, for example, data produced using the methods of the invention may be in a computer-readable, digital format for processing and interpretation by computer software. The data may thus be used to produce a data structure, also in a computer readable format, comprising counts of strings of CpG sites within a reference genome and their respective methylation states from a set of training fragments. The data may be used to generate a sample state vector, also in a computer readable format, for a sample fragment comprising a sample genomic location within the reference genome and a methylation state for each of a plurality of CpG sites in the sample fragment, each methylation state determined to be methylated or unmethylated. A plurality of possibilities of methylation states may be enumerated using a computer from the sample genomic location that are of a same length as the sample state vector. For each of the possibilities, a probability may be calculated by accessing the counts stored in the data structure. The possibility that matches the sample state vector may be identified and correspondingly the calculated probability as a sample probability. Based on the sample probability, a score may be generated for the sample fragment of the sample state vector relative to the set of training fragments. The score may be used to determine whether the sample fragment has an anomalous methylation pattern based on the generated score. The probability score can be used to make or influence a clinical decision (e.g., diagnosis of cancer, treatment selection, assessment of treatment effectiveness, etc.). For example, in one embodiment, if the likelihood or probability score exceeds a threshold, a physician can prescribe an appropriate treatment (e.g., a resection surgery, radiation therapy, chemotherapy, and/or immunotherapy).

### 6.6. Alternative Processing Steps for Adapter Ligation Prior to Capture and Enrichment of Biotin-Modified Fragments

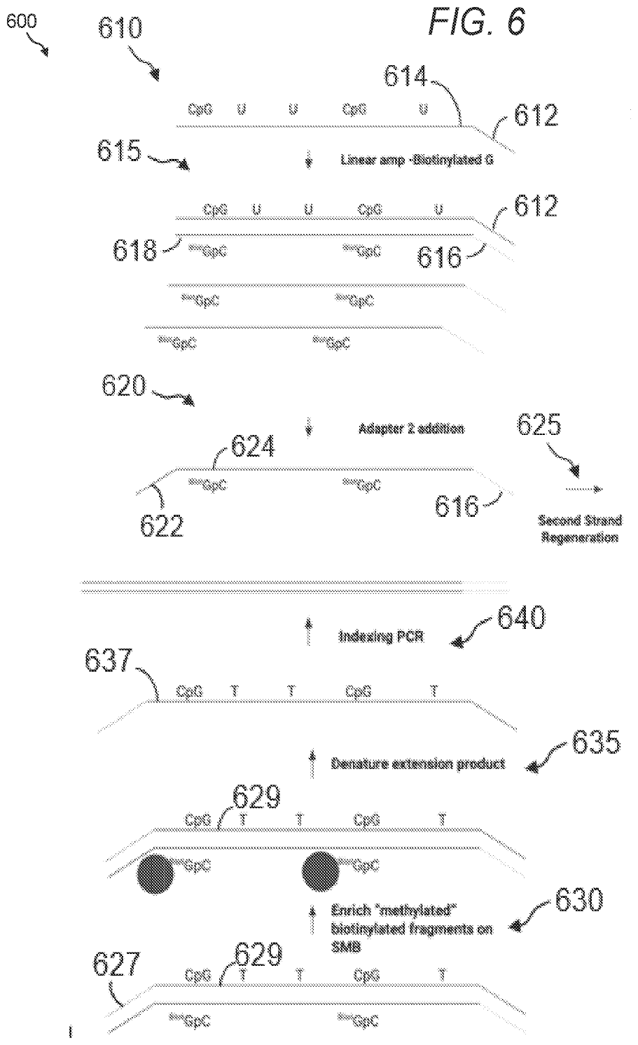

In another embodiment, ssDNA adapters can be added to the bisulfite converted ssDNA fragments obtained from step 215 of method 200 prior to capture and enrichment. FIG. 6 shows pictorially an example of certain process steps for adding ssDNA adapters to converted fragments prior to capture and enrichment.

At a step 610, a first ssDNA adapter 612 is added to the 3′-OH ends of bisulfite converted ssDNA fragments in a single-stranded DNA ligation reaction to generate converted adapter-ligated ssDNA fragments or constructs 614. In one embodiment, the first ssDNA adapter can be added to the converted ssDNA fragment as described with reference to step 510 of method 500.

At a step 615, the converted adapter-ligated ssDNA fragments 614 are copied to add in binding moiety-modified nucleotides. In one embodiment, the converted adapter-ligated ssDNA fragments can be copied to add in binding moiety-modified nucleotides as described with reference to step 220 of method 200. For example, a primer 616 that is complimentary to the first ssDNA adapter 612 can be annealed to the converted adapter-ligated ssDNA fragments 614 and extended in an amplification or primer extension reaction using a mixture of biotin-dGTP and dGTP (not shown) to produce from converted adapter-ligated ssDNA fragments 614 a copy DNA 618 in which a portion of the guanines may be biotinylated guanines (indicated here as ^{Biot}G). In one example, 20 cycles of amplification or primer extension can be used to yield 20 single-stranded copies 618 of adapter-ligated ssDNA fragments 614 with incorporated biotin-dGTP that are the compliment of the original input molecule.

At a step 620, a second ssDNA adapter 622 is added to the 3′-OH ends of copy DNA 618 using a single-stranded DNA ligation reaction. Ligation of the second ssDNA adapter generates a converted ssDNA fragment 624 that includes a first adapter and a second adapter. ssDNA fragment 624 is a reverse complement copy of the original converted fragment. In one embodiment, the second ssDNA adapter can be added to the converted ssDNA fragment as described with reference to step 510 of method 500.

At a step 625, a second strand DNA is synthesized in a primer extension reaction to generate double-stranded DNA (dsDNA) constructs. For example, a primer 627 that is complimentary to the second ssDNA adapter 622 can be annealed to converted ssDNA fragment 624 and extended in a primer extension reaction to generate double stranded DNA (dsDNA) constructs 629. In one example, a single round of a primer extension reaction may be used to generate dsDNA constructs 629, wherein the original unconverted cytosines in the original DNA molecule are now represented by thymidine (T) and methylated cytosines are CpG.

At a step 630, dsDNA constructs with incorporated biotin-dGTP are captured. In one embodiment, dsDNA constructs 629 with incorporated biotin-dGTP can be captured using a streptavidin coated solid support, such as streptavidin coated beads, as described with reference to step 225 of method 200. The output of the capture step 630 is a biotin enriched sample, i.e., the input sample has been enriched for the desired degree of methylation.

At a step 635, the dsDNA constructs in the biotin enriched sample are denatured. For example, the dsDNA constructs 629 may be denatured using a heat denaturation process or an alkali-based denaturation process to yield a converted ssDNA construct 637. The biotinylated strand of dsDNA constructs 629 remains bound to the capture surface (e.g., streptavidin coated beads).

At a step 640, the converted ssDNA constructs 637 are amplified to generate a sequencing library. In one embodiment, converted ssDNA construct 637 can be amplified in an indexing PCR reaction to generate a sequencing library as described with reference to step 515 of method 500.

### 6.7. Compositions and Kits

The disclosure includes disclosure of a variety of compositions. Any composition resulting from a method step may be a novel composition of the invention.

For example, compositions include the various mixtures of nucleotides described herein. In certain aspects, compositions include a mixture of binding moiety-modified nucleotides and binding moiety-lacking nucleotides in the various quantities described herein. In certain embodiments, compositions include a mixture of binding moiety-modified cytosines and binding moiety-lacking cytosines in the various quantities described herein. In certain embodiments, compositions include a mixture of binding moiety-modified guanines and binding moiety-lacking guanines in the various quantities described herein.

In certain aspects, compositions include a mixture of adenine, guanine, cytosine and thymine including binding moiety-modified nucleotides and binding moiety-lacking nucleotides in the various quantities described herein. In certain aspects, compositions include a mixture of adenine, guanine, cytosine and thymine including binding moiety-modified cytosines and binding moiety-lacking cytosines in the various quantities described herein. In certain aspects, compositions include a mixture of adenine, guanine, cytosine and thymine including a mixture of binding moiety-modified guanines and binding moiety-lacking guanines in the various quantities described herein.

In certain aspects, compositions include DNA molecules into which the mixtures of nucleotides have been copied. In certain aspects, compositions include mixtures of DNA molecules into which the mixtures of nucleotides have been copied. In certain aspects, compositions include mixtures of binding moiety-modified fragments and unmodified fragments. In certain aspects, compositions include mixtures of binding moiety-modified fragments and unmodified fragments wherein at least a portion of the binding moiety-modified fragments are bound to a substrate.

In certain aspects, compositions include DNA molecules enriched for hypermethylated fragments using the methods of the invention.

In certain aspects, the compositions include adenines, thymines, cytosines and guanines wherein the cytosines, guanines, or both cytosines and guanines are included in a mixture of binding moiety-modified nucleotides and binding moiety-lacking nucleotides. In certain aspects, the composition lacks or substantially lacks binding moiety-modified adenines and lacks binding moiety-modified guanines.

The compositions may in certain embodiments be provided in any suitable buffer solution.

The mixtures of binding moiety-modified nucleotides and nucleotides lacking the binding moiety may have any of the ranges described herein. For example, in one embodiment, the mixture ranges from 1 to 20 percent binding moiety-modified nucleotides with the remainder of the nucleotides lacking the binding moiety. In another embodiment, the binding moiety ranges from 2.5 to 10 percent binding moiety-modified nucleotides with the remainder of the nucleotides lacking the binding moiety.

The disclosure provides methods of making the compositions by combining the various components of the compositions. The compositions may be provided in sealed, labeled packaging.

### 6.8. Kits

The disclosure provides kits comprising any of the compositions described herein. For example, a kit may include a composition and instructions for using the composition. The instructions may, in certain embodiments, include instructions for using any of the reagents or compositions described herein to perform any of the methods described herein. A kit may include any of the reagents and compositions described herein. A kit may include reagents or other components for isolating nucleic acids. The reagents or other components for isolating nucleic acids may include a substrate, such as beads or wells, for capturing nucleic acids. A kit may include reagents for eluting nucleic acids from a substrate. A kit may include reagents for converting unmethylated cytosines of nucleic acid fragments to uracils. Reagents for converting unmethylated cytosines of nucleic acid fragments to uracils may include reagents for deaminating the unmethylated cytosines. Reagents for converting unmethylated cytosines of nucleic acid fragments to uracils may include reagents for converting by enzymatic conversion. The disclosure provides methods of making the kits by assembling the various components of the kits into common packaging.

### 6.9. Automation and Analysis

The methods of the invention may be automated using robotics or microfluidic devices. The disclosure includes software programmed to execute methods of the invention using robotics or microfluidics devices. The disclosure provides systems programmed and configured to execute the software. The software may also analyze data from a sequencing determination on enriched fragments to produce results. The analysis may be performed on a computer. The results may be provided as a report. The report may, for example, be delivered to a physician or to a subject. The report may, for example, be electronic or printed or may be delivered via any output means. A therapeutic treatment may be selected or deselected based on the results.

### 6.10. Examples

In various embodiments, the method combines incorporation of biotinylated bases and streptavidin pulldown (e.g., using streptavidin-coated beads) to enrich for hypermethylated DNA fragments. The streptavidin-biotin methylation enrichment method (referred to in the following examples as “biotin enrichment” or “biotin enriched”) may, for example, be used to enrich for methylated DNA prior to sequencing.

Several studies were designed and performed to evaluate and optimize the incorporation of the biotin enrichment process into a sequencing library preparation protocol. The samples used in the studies as input samples were “PC2” and “Input B.” Both samples, PC2 and Input B, included a defined percentage of a sample “Input A” which consists of a 50/50 mixture of fully methylated and fully non-methylated sheared genomic human HCT116 KDO DNA. PC2 consists of 2% of Input A in NA24631. NA24631 refers to sheared genomic DNA from the reference cell line NA24631 (a NIST reference cell line). Input B consists of 5% of Input A in pooled healthy cfDNA.

A standard bisulfite conversion library preparation method (referred to as V2 or V2 GMS) was used as a control method. “GMS” refers to a method that was previously developed for the preparation of next generation sequencing (NGS) libraries from bisulfite-converted DNA or any single-stranded DNA. V2 refers to a version of the standard bisulfite conversion protocol.

A biotin enrichment library preparation process may include several unique steps. For example, the biotin enrichment library preparation process may include a linear amplification step, a strand regeneration step, and a biotinylated DNA capture step (e.g., streptavidin bead pulldown step) as described hereinabove with reference to FIG. 6.

The linear amplification reaction can be used to incorporate biotinylated-dGTP (biotin-dGTP or biotin-G) into bisulfite converted DNA. To accomplish this, a modified standard V2 GMS linear amplification process can be used. An example of a modified linear amplification reaction for incorporating biotin-dGTP into bisulfite converted DNA is shown in Table 1.

TABLE 1
Example 10% biotin-dGTP biotin enrichment
linear amplification reaction condition.

| | Volume (μL) | Final Concentration | |
|---|---|---|---|
| Reagent | per Sample | in Reaction | |
| 5x VeraSeq buffer II | 10 | 1x | |
| dNTP mix (25 mM) | 0.5 | 250 | uM |
| without dGTP | | | |
| biotin-dGTP (1 mM) | 1.25 | 25 | uM |
| dGTP (1 mM) | 11.25 | 225 | uM |
| VeraSeq Ultra (2 U/μL) | 0.5 | 0.02 | U/μL |
| GMS Extension Primer (100 uM) | 1 | 2 | uM |
| RSB (10 mM Tris, pH 8.0) | 1.5 | | |
| DNA Input | 24 | | |
| Total | 50 | | |

The strand regeneration step can be used to make a copy containing both adapter sequences (i.e., DNA with the first and second adapter attached) into double stranded DNA for use in the biotin enrichment reaction. An example of a strand regeneration reaction is shown in Table 2. The accompanying thermocycling paraments for the example strand regeneration reaction are shown in Table 3.

TABLE 2
Example biotin enrichment strand regeneration reaction.

| | Volume (μL) | Final Concentration |
|---|---|---|
| Reagent | per Sample | in Reaction |
| Double ligated DNA | 31 | |
| KAPA HiFi HotStart ReadyMix (2X) | 40 | 1x |
| Strand Regeneration Primer | 10 | 3 μM |
| (25 μM) | | |
| Total | 50 | |

TABLE 3
Example biotin enrichment strand regeneration
thermocycling parameters (heated lid, 105° C.).

| | | |
|---|---|---|
| Temperature | Duration | Cycles |

| | | | |
|---|---|---|---|
| 98° C. | 1 | minute | 1 cycle |
| 60° C. | 30 | seconds | 1 cycle |
| 72° C. | 90 | seconds | 1 cycle |

| | |
|---|---|
| 4° C. | hold |

The strand regeneration reaction may be followed by a post-strand regeneration cleanup step. In one example, the post-strand regeneration cleanup step consists of a standard 1.4× SPRI cleanup procedure with a 25 μL elution that can be used directly in a biotinylated DNA capture reaction. The main purpose of this step is for buffer exchange (removing unincorporated nucleotides/primers, salts, and enzymes) and volume reduction (81 μL initial to 25 μL final) to facilitate the biotinylated DNA capture reaction.

To capture and enrich the biotinylated fragments, a standard enrichment protocol using streptavidin magnetic beads (SMBs) may be used. An example of a SMB capture reaction for enrichment of biotinylated fragments is shown in Table 4. In one example, DNA from the post-strand regeneration cleanup step is combined with the SMBs and incubated at room temperature for 30 minutes. Following the incubation period, the beads with bound biotinylated fragments thereon, are washed twice with 200 μL of a 1× bind and wash ((1×B+W) buffer, (5 mM Tris-HCl (pH 7.5)+0.5 mM EDTA+1M NaCl). The bound DNA is eluted from the SMBs using 16.8 μL of elution buffer (0.1M NaOH diluted in Hybridization Elution Buffer (HEB1) and neutralized with 3.2 μL of Hybridization Neutralization Buffer (HNB1). The eluted DNA can be used as input in a sequencing library indexing PCR reaction. An example of a sequencing library indexing PCR reaction is shown in Table 5. The accompanying thermocycling paraments for the indexing PCR reaction are shown in Table 6. After the indexing PCR reaction, a 1×SPRI cleanup may be performed to complete the biotin enrichment library preparation process.

TABLE 4
Example biotin enrichment DNA hybridization capture reaction.

| | Volume (μL) | Final Concentration |
|---|---|---|
| Reagent | per Sample | in Reaction |
| DNA from post-strand regeneration | 25 | |
| cleanup elution | | |
| Hybridization Capture Buffer (HB1) | 25 | 1x |
| SMB | 130 | |
| Total | 50 | |

TABLE 5
Example biotin enrichment library indexing PCR reaction.

| | Volume (μL) | Final Concentration |
|---|---|---|
| Reagent | per Sample | in Reaction |
| DNA input | 20 | — |
| KAPA HiFi HotStart ReadyMix | 25 | 1x |
| (2X) | | |
| 5x diluted Indexing primer mix | 5 | 0.5 μM |
| (5 μM Concentration Total, | | |
| 2.5 μM each index i5 i7) | | |
| Total | 50 | |

TABLE 6
Example biotin enrichment library indexing PCR
thermocycling parameters (heated lid, 105° C.).

| | | |
|---|---|---|
| Temperature | Duration | Cycles |

| | | | | |
|---|---|---|---|---|
| 98° C. | 45 | seconds | 1 | cycle |
| 98° C. | 15 | seconds | 10 | cycles |
| 60° C. | 30 | seconds | | |
| 72° C. | 30 | seconds | | |
| 72° C. | 1 | minute | 1 | cycle |

| | |
|---|---|
| 4° C. | hold |

Simulation studies were performed to evaluate using incorporation of biotin-dGTP (or biotin-dGTP) and subsequent enrichment of biotin-modified fragments on assay performance and workflow.

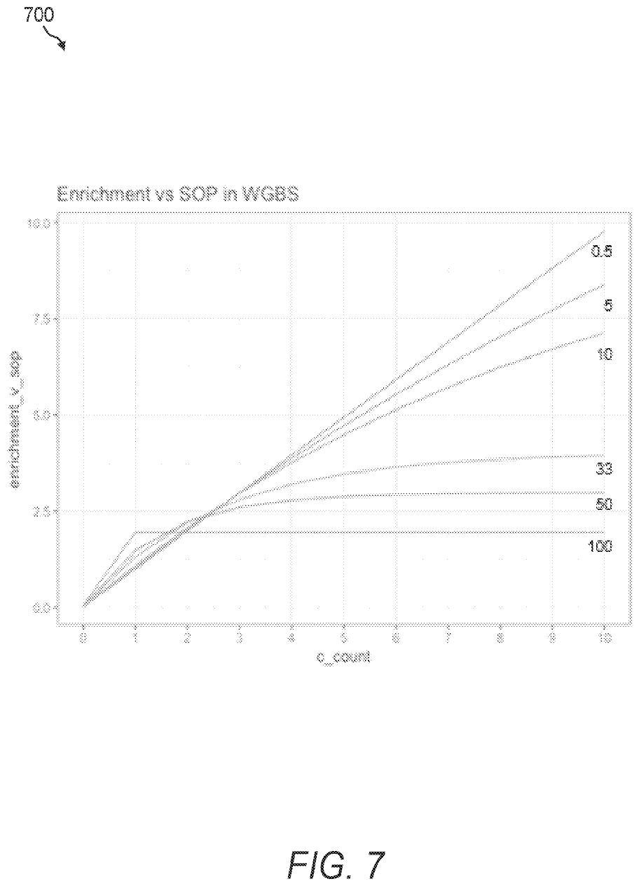

For example, since the number of methylated cytosines in a fragment can vary based on sequence composition and length, we anticipated that labeling methylated cytosines with complementary biotin-dGTP will be dependent on the biotin-dGTP concentration ratio (i.e., percent biotin-dGTP in a dNTP mix). FIG. 7 is a plot 700 showing the expected fold enrichment based on simulations involving various biotin-dGTP percentages (0.5, 5, 10, 33, 50 and 100). The simulation data shows that sensitivity and specificity for methylated fragments can be controlled by adjusting the biotin-dGTP ratio.

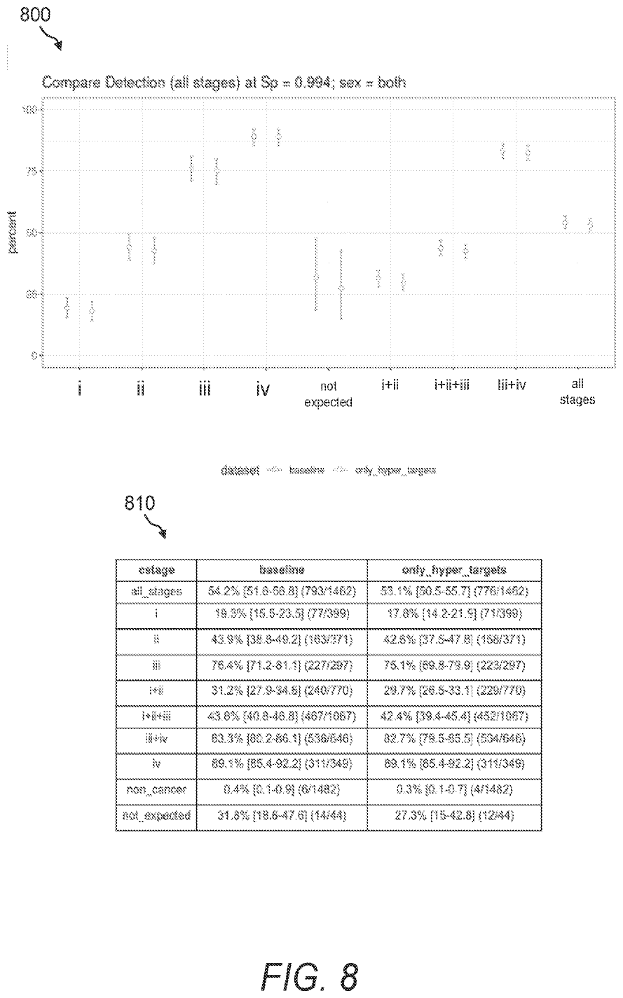

In some applications, libraries enriched for methylated fragments may be used in a sequencing cancer testing or screening protocol. A simulation was performed to evaluate using only hypermethylated targets in a testing or screening protocol. FIG. 8 is a plot 800 and a table 810 showing cancer classification performance for only hypermethylated targets (left in each pair) vs all Compass (baseline) targets (right in each pair). Compass is a target enrichment panel. This analysis shows that a subset of targets within that enrichment panel representing only those where hypermethylation is associated with cancer can be selected. The panel is very large, and many subsets of the target set can recapitulate the performance of the entire panel. Based on simulations, we determined that using only hypermethylated targets may achieve similar cancer classification performance as total Compass targets.

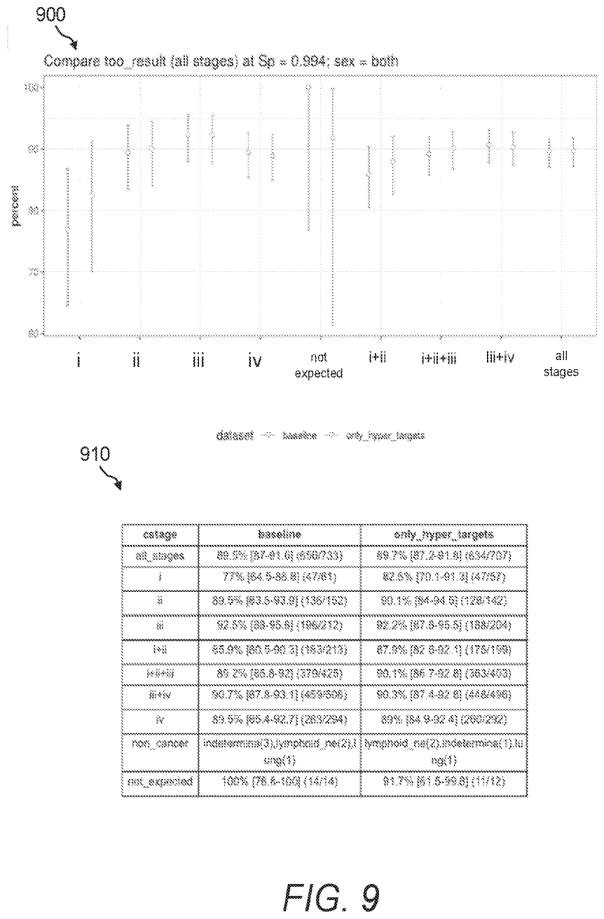

FIG. 9 is a plot 900 and a table 910 showing cancer signal origin (CSO) classification performance for only hypermethylated targets (left in each pair) vs all Compass (baseline) targets (right in each pair). Based on simulations, using only hypermethylated targets may achieve similar tissue of origin (TOO) classification performance as total Compass targets.

In addition to comparable classification performance, the use of biotinylated-dGTP (or biotinylated-dCTP) and enrichment of modified fragments may improve abnormal hypermethylation coverage since it directly captures and targets methylated fragments, which may help to improve, for example, ctDNA coverage in these hypermethylated regions.

Furthermore, since the biotin-labeling and subsequent streptavidin pulldown is essentially pre-enriching for hypermethylated fragments, overall library complexity should be reduced. Reduced library complexity has the potential to reduce sequencing depth requirements and thereby reduce the cost of goods (COGs), facilitate higher signal to noise for cancer signals, and allow for less stringent enrichment hybridization reactions (i.e., target enrichment using 1 or 2 hybridization enrichment steps with shortened durations) while maintaining assay performance and improving assay workflow and turnaround time (TAT).

### 6.10.1. Proof of Concept Experiment

To determine the feasibility of enriching for hypermethylated fragments using biotinylated bases and streptavidin pulldown and incorporating the process into a standard bisulfite conversion (BSC) sequencing library preparation process (V2 GMS), we designed and executed a proof of concept (POC) experiment. This POC experiment served as a first pass at introducing several new process specific steps into the V2 BSC sequencing library preparation process. V2 is an automated target methylation sequencing test system that has been used to detect methylation patterns in plasma circulating cell-free DNA. Briefly, the V2 library preparation process includes the steps of bisulfite conversion, ligation of a first adapter, linear amplification of the adapter ligated DNA to generate double-stranded DNA, ligation of a double stranded second adapter, indexing PCR amplification, hybridization enrichment of target specific sequences, and sequencing. The target enrichment step in the V2 protocol includes two rounds of hybridization to an enrichment panel of target specific probes (i.e., the prepared libraries are hybridized to the enrichment panel, eluted form the panel, and re-hybridized to the enrichment panel a second time).

To merge the two processes, the following changes may be incorporated:
Including biotin-dGTP into the linear amplification step to tag for methylated fragments,
A second strand regeneration step, after 2nd adapter ligation, that uses a primer complementary to the 2nd adapter to generate double stranded DNA (dsDNA) for input into streptavidin magnetic bead (SMB) pulldown and capture of the biotin-dGTP modified (methylated) fragments,
A post-strand regeneration SPRI cleanup for buffer exchange and volume reduction,
A SMB pulldown to enrich for our biotinylated dsDNA target,
Additional SMB enrichment washes (two in total) to help reduce and remove the amount of off-target, non-biotinylated, and/or hypomethylated DNA fragments,
NaOH elution of the SMB enriched DNA to release the DNA bound to the beads, and
Biotin enrichment PCR conditions which use reduced indexing primer concentrations ( 1/10th of V2 GMS) and a more stringent post-PCR SPRI cleanup (1× instead of 1.4×) to help reduce dimers.

Biotin enriched sequencing libraries were prepared using dNTP mixes comprising different percentages of biotin-dGTP and various PCR amplification cycles. The V2 GMS library preparation process was used as a control method. Libraries were characterized, sequenced and the data were analyzed for various metrics.

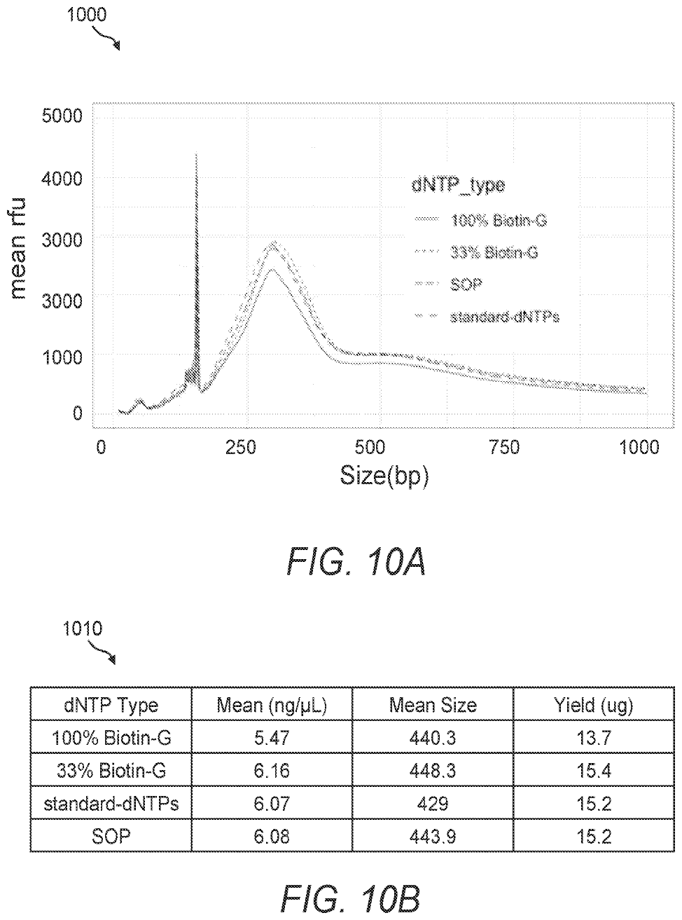

To integrate the use of biotinylated dNTPs in the linear amplification step of the V2 GMS library preparation process, libraries were generated using dNTP mixes comprising 100% biotin-G, 33% biotin-G, a standard-dNTP mix, or an SOP mix. The resulting library products were run on an NGS Fragment Analyzer to determine the compatibility of incorporating biotinylated bases into the library preparation process. FIG. 10A is a plot 1000 showing the Fragment Analyzer profiles for the libraries prepared using different dNTP mixes. FIG. 10B is a table 1010 showing yields for the libraries prepared using the different dNTP mixes. The data show that the library profiles and yields for the various dNTP conditions used are similar.

We also evaluated different sources/vendors for biotin-dGTP, a broad range of biotin-dGTP percentages for dNTP mix supplementation, and various PCR amplification cycles to further optimize the biotin enrichment library preparation process. For this experiment, 12.5 ng of Input B was used as starting material in a manual BSC reaction and libraries were manually prepared as described in Table 7. The standard BSC sequencing library preparation process (V2 GMS) was used as a control. In the examples that follow, control libraries are designated as V2 SOP or SOP.

TABLE 7
Proof of concept experiment library conditions

| | | | | | IndexingPCR | No. of |
|---|---|---|---|---|---|---|
| Library Type | Condition Description | Vendor | dNTP Mix | | Cycles | Samples |
| V2 GMS | SOP control | SOP | SOP (0% | biotin-G) | 14 | 6 |
| Control | Standard dNTP SOP | Standard | Standard (0% | biotin-G) | 14 | 1 |
| | mix Control | | | | | |
| Biotin | PerkinElmer biotin-G | PerkinElmer | 33% | biotin-G | 10 | 1 |
| Enriched | dNTP mix and PCR | | | | 14 | 2 |
| | cycle testing | | 100% | biotin-G | 14 | 1 |
| | | | | | 17 | 1 |
| | Trilink biotin-G | Trilink | 10% | biotin-G | 10 | 1 |
| | dNTP mix and PCR | | | | 14 | 1 |
| | cycle testing | | 33% | biotin-G | 10 | 1 |
| | | | | | 14 | 1 |
| | | | 100% | biotin-G | 10 | 1 |
| | | | | | 14 | 1 |

To assess the relative CpG enrichment in the libraries, whole-genome bisulfite sequencing (WGBS) on a Novaseq S2 FC (18 samples/FC depth) was performed. Data analysis was performed using methyl_3.14.2-wgbs_cfdna_no_trimming_siege and methyl_3.14.2-targeted_cfdna_Compass pipelines.

The resulting libraries were enriched using the Compass targeted methylation (TM) enrichment panel. The libraries were sequenced on a Novaseq S2 FC @ 18 samples/FC. Data analysis was performed using methyl_3.14.2-targeted_cfdna_Compass and methyl_3.14.2-targeted_cfdna_Compass_custom to 75 million reads pipelines in order to examine overall analytical assay performance for a variety of key characteristic metrics.

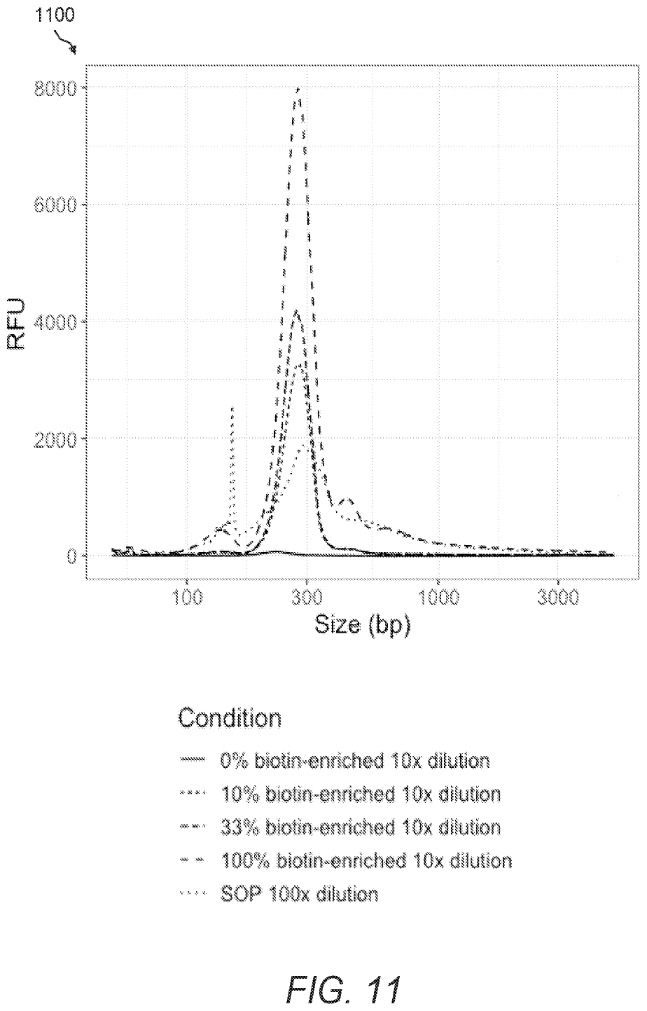

FIG. 11 is a plot 1100 showing Fragment Analyzer library profile comparisons for V2 GMS control and biotin enriched libraries prepared using the conditions shown in Table 7. Pre-sequencing metrics indicate that the biotin enrichment library protocol is highly specific for biotin-modified DNA but generates slightly shorter library fragments compared to the V2 GMS protocol as illustrated by the Fragment Analyzer traces. The library profile distribution, as illustrated by the Fragment Analyzer traces, for the biotin enriched libraries are narrower and shifted to the left towards shorter fragments with a peak height around 275 bps whereas the control libraries (SOP) tend to be broader and centered around 300 bps. The number of dimers, peaks around 154 bps, is lower in the biotin enriched libraries relative to the control libraries (SOP). In the absence of biotin labeling, the biotin enriched libraries are flat and fail library preparation due to the absence of target molecules.

In addition, biotin enriched library yields are lower than the control libraries (V2 GMS controls) as shown in Table 8. Higher percentages of biotin translated to higher library yields. However, V2 SOP libraries had library yields 16 μg compared to (at highest) 2.5 μg for the biotin enriched libraries. Libraries generated with unmodified non-biotinylated DNA (0% biotin enriched condition) essentially had zero yield.

TABLE 8
Fragment Analyzer (FA) library yield comparisons
for V2 GMS control and biotin enriched libraries

| Sample | FA (ng/μL) | FA Library Yield (ng) |
|---|---|---|
| 0% biotin enriched 10x | 0.077 | 18.5 |
| 10% biotin enriched 10x | 3.397 | 815.3 |
| 33% biotin enriched 10x | 3.788 | 909.0 |
| 100% biotin enriched 10x | 10.704 | 2569.0 |
| V2 SOP 100x | 6.9 | 16603.8 |

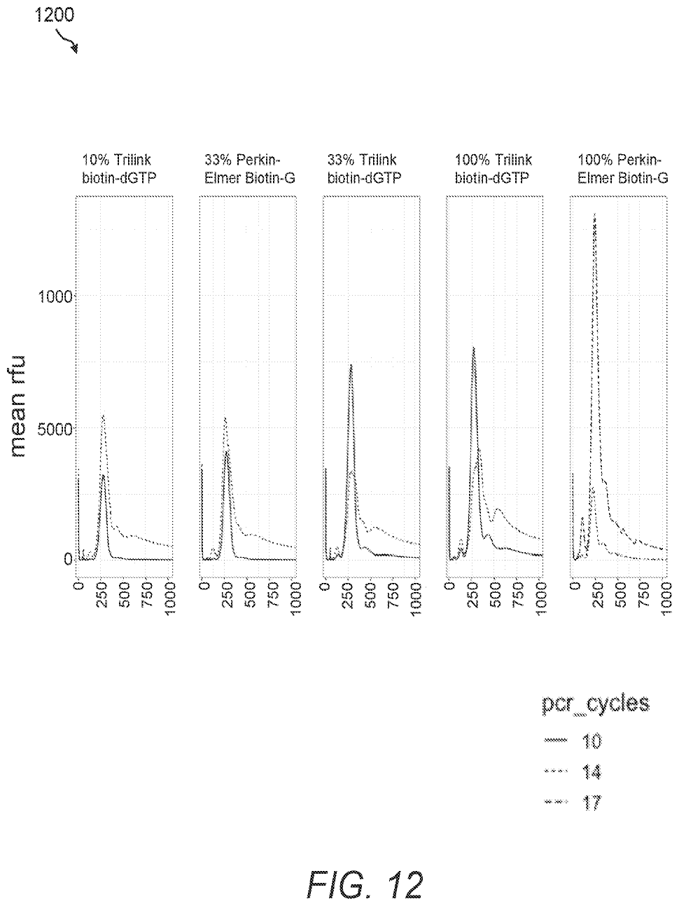

Library preparations for each biotin percentage and dNTP mix combination (see Table 7) were evaluated by indexing PCR cycles to determine the number of cycles that balances library yield and generation of artifacts. FIG. 12 is a panel of plots 1200 showing library profile comparisons for the biotin enriched libraries prepared using 10, 14, and 17 PCR cycles by percent biotin utilized. The data show that 10 cycles of PCR allows for the use of a broader range of percentages of biotin-dGTP mixes without over amplifying the libraries and generating artifacts (e.g., as much bubble DNA which peaks at and around 500+bps) as observed in the 14 and/or 17 PCR cycle biotin enrichment library preparations.

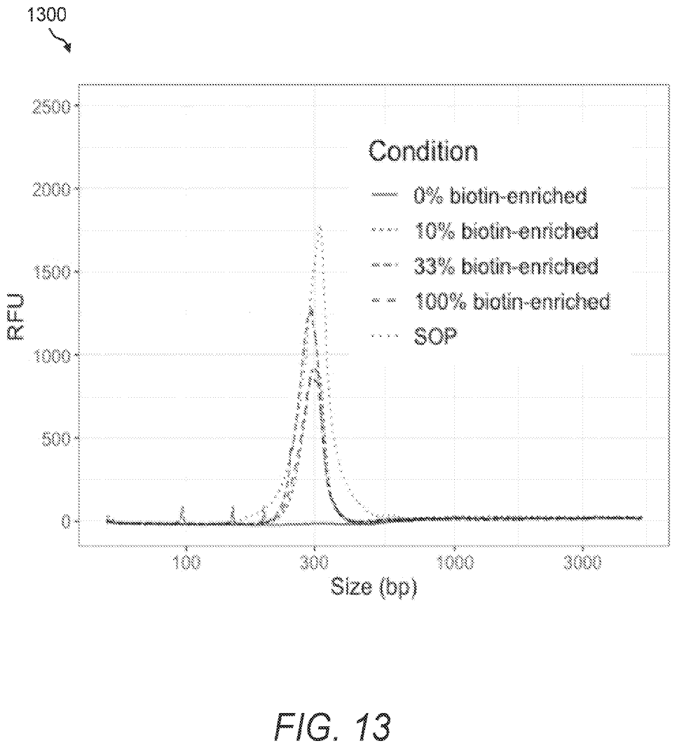

Further, target enrichment (using the Compass panel) of the biotin enriched libraries yielded sufficient concentrations (>2 nM) for sequencing. FIG. 13 is a plot 1300 showing target enriched library profiles for the V2 SOP and biotin enriched libraries. The data show that the biotin enriched library profiles are reasonable, albeit somewhat shorter than the control library (V2 SOP). Library yields (as determined by qPCR) for the target enrichment samples are shown in Table 9.

TABLE 9
qPCR enrichment yields

| Sample | qPCR Yield (nM) |
|---|---|
| V2 SOP | 87.7 |
| 10% Biotin enriched | 40.3 |
| 33% Biotin enriched | 66 |
| 100% Biotin enriched | 43.5 |

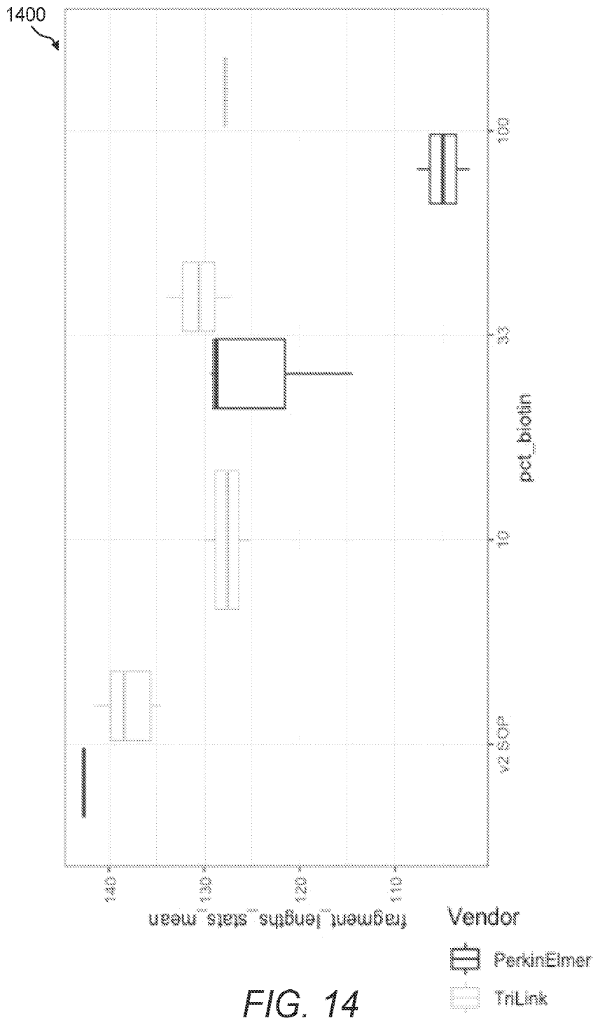

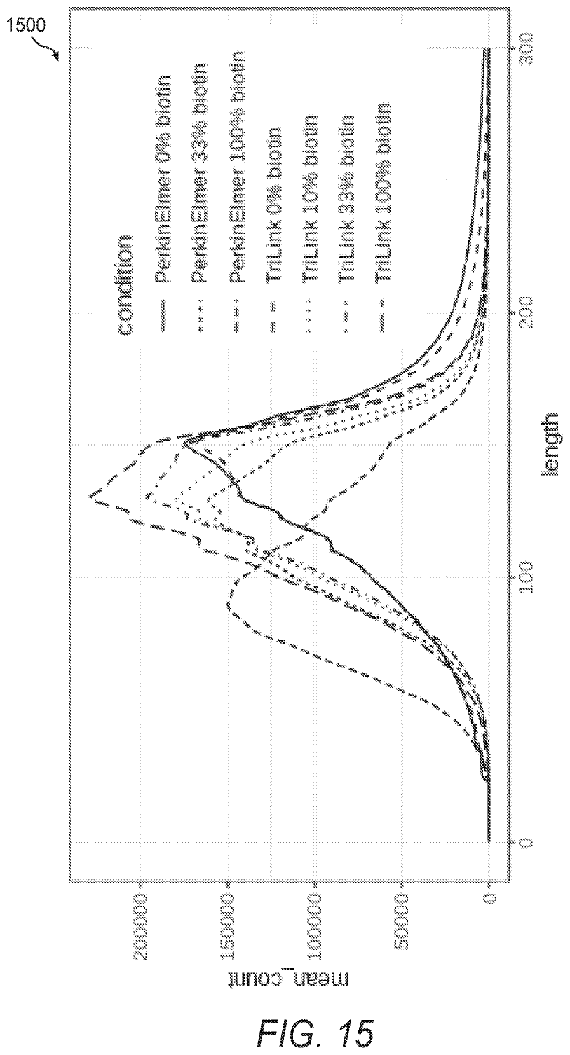

The mean fragment lengths and fragment distributions are shorter (compared to the V2 SOP library) for the biotin enriched libraries. FIG. 14 is a plot 1400 showing a comparison of the mean fragment length by percent biotin-dGTP and biotin-dGTP vendor source for the biotin enriched and V2 SOP control libraries prepared using conditions shown in Table 7. A summary of the fragment lengths in libraries prepared using different biotin-dGTP percentages and vendor sources is shown in Table 10. FIG. 15 is a plot 1500 showing the sequencing fragment distributions in the libraries prepared using different biotin-dGTP percentages and vendor sources.

Referring to FIG. 14, FIG. 15, and Table 10, the data show that fragment lengths and distributions shift to the left towards smaller fragments with peak sizes at 130 bp for the biotin enriched libraries instead of the typical 140 bp seen in V2 control libraries. The shift in size is irrespective of biotin-dGTP vendor. Fragment sizes are more consistent and less variable for Trilink biotin-dGTP.

TABLE 10
Summary of fragment lengths for libraries prepared using
different biotin-dGTP percentages and vendor source.

| Percent | | Fragment Lengths | Fragment Lengths |
|---|---|---|---|
| Biotin | Vendor | Stats Mean Mean | Stats Mean SD |
| V2 SOP | PerkinElmer | 142.63 | NA |
| V2 SOP | TriLink | 137.97 | 2.84 |
| 10 | TriLink | 127.59 | 3.4 |
| 33 | PerkinElmer | 124.17 | 8.43 |
| 33 | TriLink | 130.56 | 4.8 |
| 100 | PerkinElmer | 104.97 | 3.91 |
| 100 | TriLink | 127.82 | 0.4 |

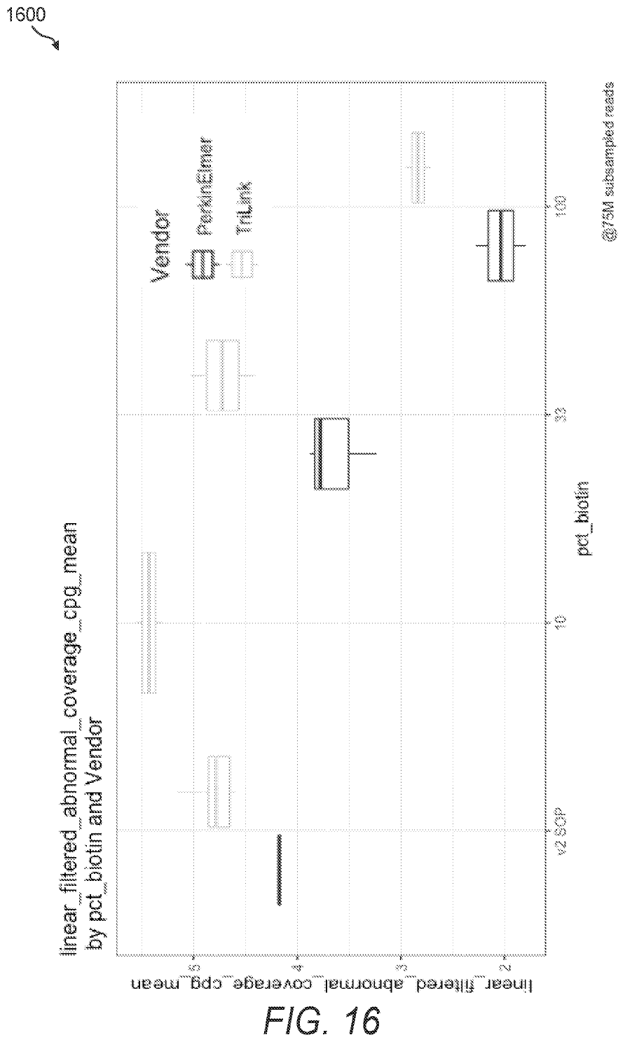

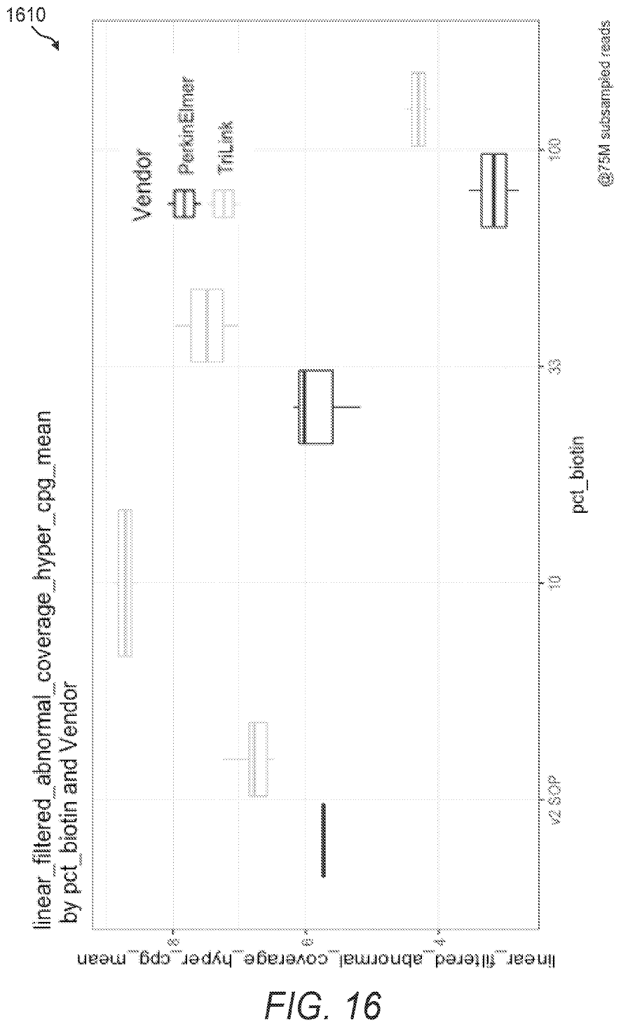

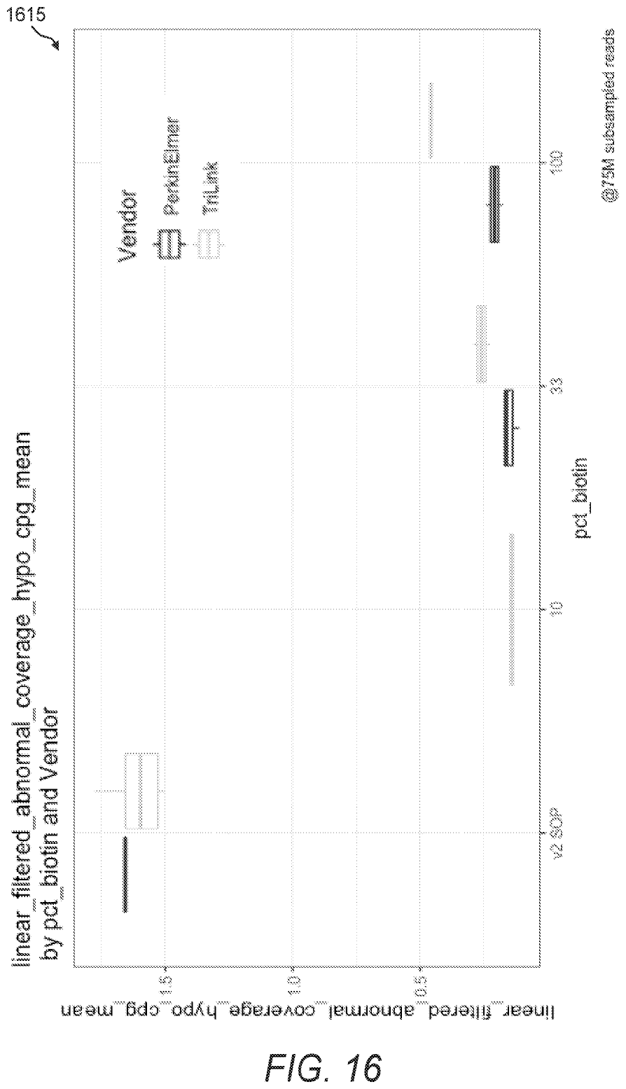

FIG. 16 is a panel of plots 1600, 1610, and 1615 showing the mean linear filtered abnormal coverage by target region for total (coverage), hypermethylated (hyper), and hypomethylated (hypo) targets, respectively, for the biotin enriched and V2 SOP control libraries prepared using conditions shown in Table 7. A summary for the linear filtered abnormal coverage metrics is shown in Table 11.

TABLE 11
Summary stats table for linear filtered abnormal coverage metrics

| Percent | | *CPG Mean | *Hyper CPG | *Hypo CPG | *CPG Mean | *Hyper CPG Mean | *Hypo CPG |
|---|---|---|---|---|---|---|---|
| Biotin | Vendor | Mean | Mean Mean | Mean Mean | SD | SD | Mean SD |
| V2 SOP | PerkinElmer | 4.17 | 5.73 | 1.66 | NA | NA | NA |
| V2 SOP | TriLink | 4.8 | 6.78 | 1.61 | 0.21 | 0.28 | 0.1 |
| 10 | TriLink | 5.43 | 8.72 | 0.14 | 0.18 | 0.28 | 0.01 |
| 33 | PerkinElmer | 3.63 | 5.79 | 0.15 | 0.35 | 0.55 | 0.03 |
| 33 | TriLink | 4.72 | 7.49 | 0.26 | 0.44 | 0.68 | 0.05 |
| 100 | PerkinElmer | 2.03 | 3.17 | 0.21 | 0.34 | 0.53 | 0.04 |
| 100 | TriLink | 2.83 | 4.31 | 0.46 | 0.17 | 0.28 | 0 |

*= Linear filtered abnormal coverage

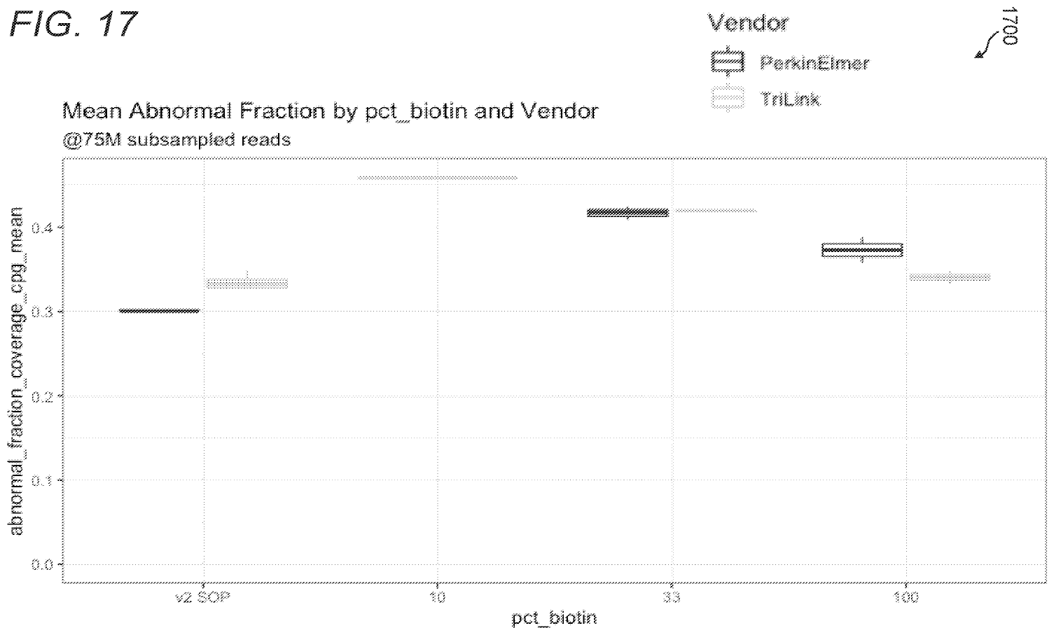

FIG. 17 is a plot 1700 showing a mean abnormal fraction comparison at 75 million subsampled reads for the biotin enriched and V2 SOP control libraries prepared using conditions shown in Table 7. A summary for the abnormal fraction coverage CPG mean metric is shown in Table 12.

TABLE 12
Summary stats table for abnormal fraction
coverage CPG mean metric.

| | | Abnormal Fraction | Abnormal Fraction |
|---|---|---|---|
| Percent | | Coverage | Coverage |
| Biotin | Vendor | CPG Mean | CPG Mean SD |
| V2 SOP | PerkinElmer | 0.3 | NA |
| V2 SOP | TriLink | 0.33 | 0.01 |
| 10 | TriLink | 0.46 | 0 |
| 33 | PerkinElmer | 0.42 | 0.01 |
| 33 | TriLink | 0.42 | 0 |
| 100 | PerkinElmer | 0.37 | 0.02 |
| 100 | TriLink | 0.34 | 0.01 |

Referring now to FIG. 16, FIG. 17, Table 11, and Table 12, the data show that the abnormal coverage means and mean abnormal fraction are the highest for the 10% biotin condition and is mostly driven by hypermethylated fragments. The 10% biotin condition gives a good balance of sensitivity and specificity for hypermethylated targets. For this condition, the total abnormal coverage (hyper+hypo methylated) is highest while maintaining the highest hypermethylation and lowest hypomethylation coverages. Thus, the overall efficiency and total abnormal fraction ratio is better for the 10% condition since it is more effective at enriching for hypermethylated targets and depleting hypomethylated targets. The other higher percentage conditions (33% and 100%) are less efficient most likely because they are mislabeling and pulling down a higher proportion of unconverted cytosine fragments (false positives) instead of the target methylated cytosine fragments. Trilink dNTPs tended to be more consistent and outperform PerkinElmer's dNTPs in these metrics.

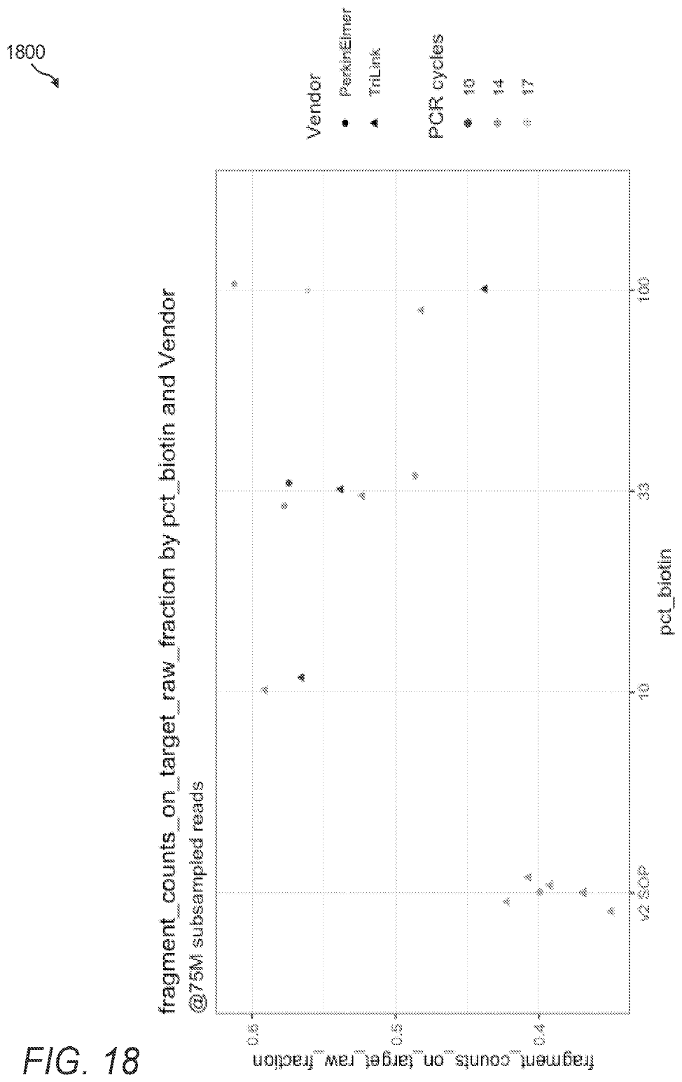

The on-target rate metric for each library was examined as an indirect way to evaluate the complexity of each library. In general, enriching for specific sequences (e.g., target enrichment hybridization) tends to be more efficient in less complex libraries. FIG. 18 is a plot 1800 showing an on-target raw fraction comparison between the V2 SOP and biotin enriched libraries prepared using conditions shown in Table 7. A summary for the fragment counts on-target raw fraction metrics is shown in Table 13.

TABLE 13
Summary table for fragment counts on-target raw fraction metrics.

| | | Fragment Counts on Target | Fragment Counts on Target |
|---|---|---|---|
| Percent Biotin | Vendor | Raw Fraction Mean | Raw Fraction SD |
| V2 SOP | PerkinElmer | 0.4 | NA |
| V2 SOP | TriLink | 0.38 | 0.03 |
| 10 | TriLink | 0.58 | 0.02 |
| 33 | PerkinElmer | 0.55 | 0.05 |
| 33 | TriLink | 0.53 | 0.01 |
| 100 | PerkinElmer | 0.59 | 0.04 |
| 100 | TriLink | 0.46 | 0.03 |

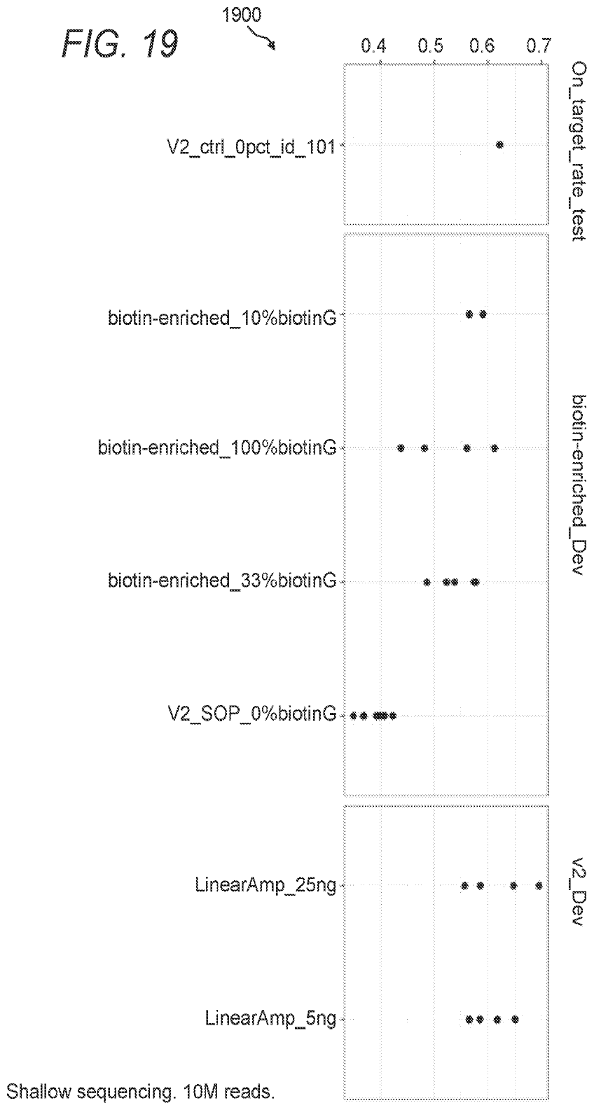

We also compared on-target rates for sequencing data from libraries prepared using a manual target enrichment process and the automated enrichment process (V2_Dev). FIG. 19 is a panel of plots 1900 showing a comparison of sequencing fragment counts for on-target rates for sequencing data from libraries prepared using the automated V2 GMS target enrichment process and a manual target enrichment process. The “On_target_rate_test” experiment (left panel) and “V2_Dev” (right panel) are data generated using the fully automated V2 GMS process whereas the “Biotin-Enriched_Dev” experiment (center panel) utilizes the manual process. Comparing the on-target rates for these V2 controls across the three experiments, we observed that the rates are similar between the two automated processes, but differ between automation and manual, with manual being lower.

Referring now to FIG. 18, FIG. 19, and Table 13, the on-target rates are higher for biotin enriched libraries than V2 GMS controls within the batch, but comparable and in-line with previously observed rates for V2 GMS controls (see FIG. 19) at 60%. The on-target rates for the V2 GMS controls are lower for the manual enrichments due to lower hybridization temperatures (58° C. vs 62° C. for automation) and less stringent washes (50° C. vs 55° C. for automation) for the typical historical automated enrichments. There is no apparent trend in and effect of the percentage of biotin, PCR cycles, and/or vendors on the target rates.

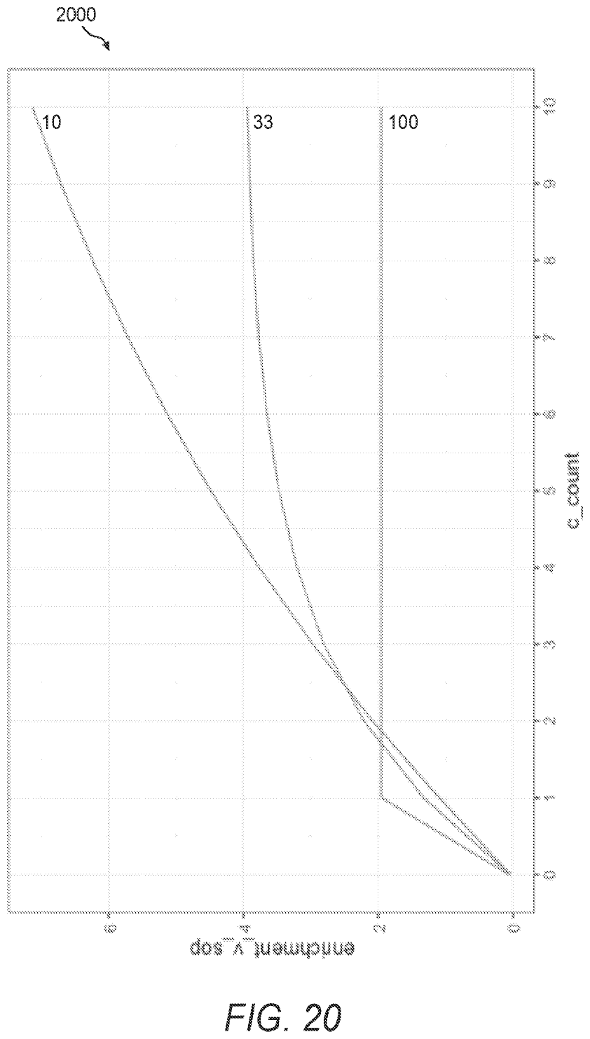

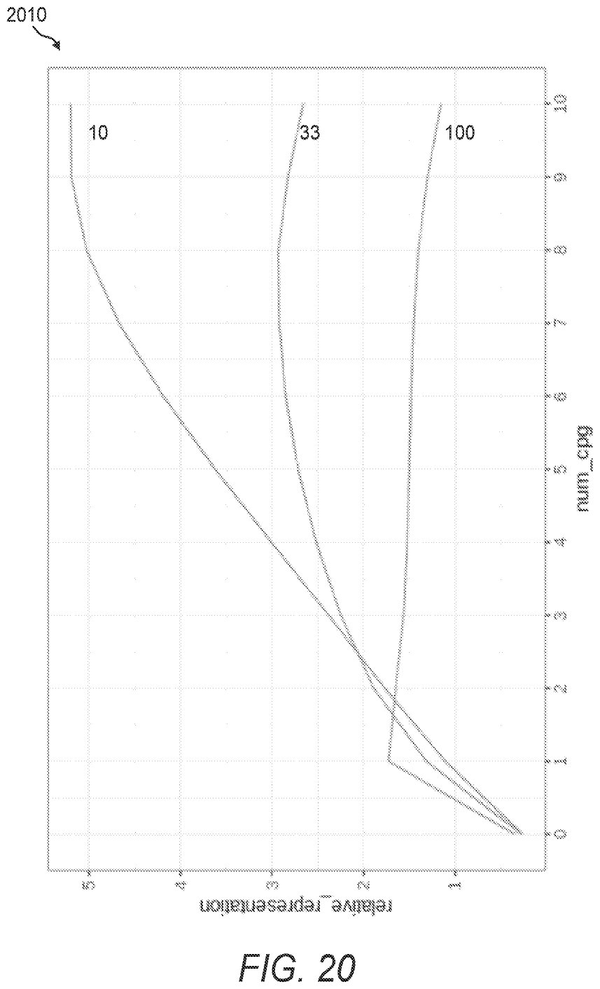

FIG. 20 is a pair of plots 2000 and 2010 showing a comparison of CpG enrichment in simulated data and WGBS data, respectively, from biotin enriched libraries relative to V2 SOP libraries. Comparing the CpG enrichment of biotin-enriched libraries relative to the V2 SOP in whole-genome bisulfite sequencing (plot 2010) and simulations (plot 2000), we observe that the relative CpG enrichment in biotin enriched libraries with respect to V2 SOP is close to the simulated data. Percent biotin is shown at 10, 33 and 100.

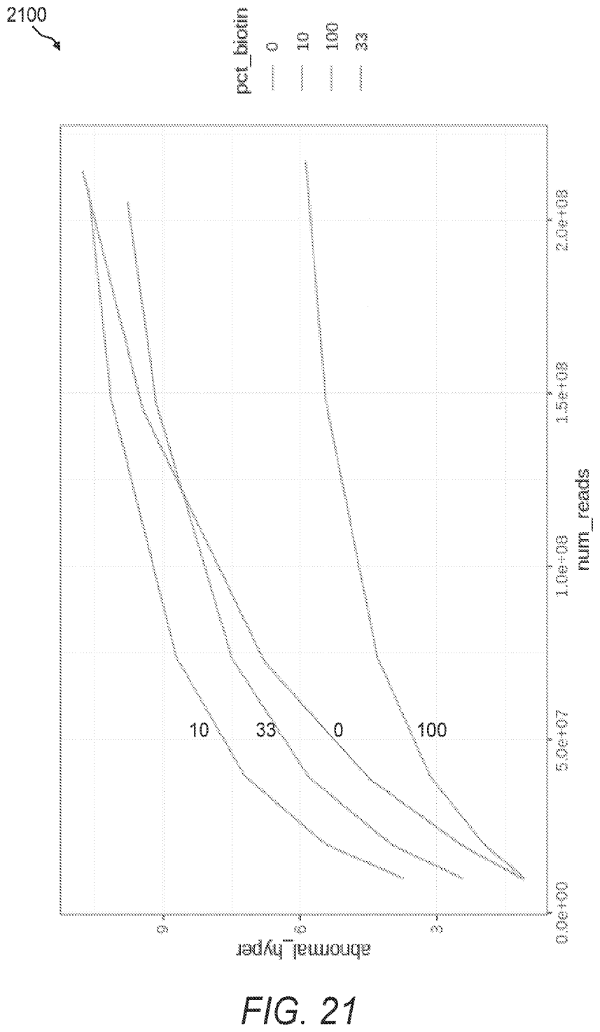

FIG. 21 is a plot 2100 showing abnormal hypermethylation coverage by sequencing depth for the biotin enriched and V2 control libraries. The data show that the biotin enriched libraries (pct_biotin=10 and 33) are more sequencing efficient since less depth is needed to achieve equivalent abnormal hyper coverage vs the V2 control (pct_biotin=0).

Based on this proof of concept (POC) experiment, the biotin enrichment library preparation process is feasible and enriches for hypermethylated fragments. Biotin enriched libraries generated acceptable pre-sequencing and sequencing results with respect to V2 GMS controls. Utilization of biotin-dGTP incorporation and labeling of bisulfite converted fragments is compatible and can be integrated with the standard V2 GMS library preparation process. TriLink biotin-dGTP may be used for future experiments because of its more consistent performance.

However, biotin enriched libraries tend to be shorter than their V2 GMS counterparts. This observation was both unexpected and undesirable since longer fragments tend to be more informative. In addition to the shorter fragment lengths, library yields were also substantially lower for the biotin enriched libraries which may introduce problems in the library enrichment process, e.g., insufficient inputs into enrichment can negatively impact performance.

### 6.10.2. Improving Library Fragment Recovery in Biotin Enriched Libraries

The proof-of-concept (POC) experiment showed that the biotin enrichment library preparation protocol generates libraries of lower yields with shorter library profiles and sequencing fragments in comparison with V2 control libraries. The lower yields were expected since this assay excludes hypomethylated fragments. However, the shorter fragment lengths were unexpected and concerning since potential target molecules may be lost. Several experiments were performed to evaluate and improve library fragment recovery in the biotin enrichment library preparation protocol.

In the POC biotin enrichment protocol, a high salt buffer (1×B+W) that included 1M NaCl was used as the washing buffer for the capture reactions with streptavidin beads. We hypothesized that high salt carryover from the 1×B+W buffer may be inhibiting the PCR reactions and causing the lower yields and shorter fragments that we observed. To test this hypothesis, we modified the original biotin enrichment process (“Biotin-Enriched_origninal”) used in the POC experiment to as follows: (i) an additional RSB rinse step was included prior to DNA elution (“Biotin-Enriched_RSB”), (ii) replaced the 1×B+W buffer (“Biotin-Enriched_original”) with a hybridization enrichment wash buffer (HEB; “Biotin-Enriched_HEB”), and (iii) used V2 SOP libraries as controls (“V2_ctrl”). In addition, for this experiment we used 12.5 ng of Input B as the starting material for a manual bisulfite conversion reaction and manually prepared libraries as detailed and described in Table 14. The libraires were evaluated on an NGS Fragment Analyzer for library profile distributions and yields.

TABLE 14
Experimental conditions for improving biotin enrichment library fragment recovery.

| | Library | dNTP | N | Purpose and |
|---|---|---|---|---|
| Condition | Prep Method | Mix | Samples | Description |
| V2_ctrl | V2 GMS | SOP | 3 | V2 GMS Baseline Control |
| V2_ctrl | V2 GMS | 10% | 1 | V2 GMS Baseline Control |
| | | biotin-G | | using 10% biotin-G mix |
| Biotin- | Biotin Enrichment | SOP | 1 | Biotin Enrichment Negative |
| Enriched_original | | | | Control |
| Biotin- | Biotin Enrichment | 10% | 3 | Biotin Enrichment Baseline |
| Enriched_original | | biotin-G | | (Positive) Control |
| Biotin- | Biotin-Enriched_rsb | SOP | 1 | Biotin-Enriched_rsb |
| Enriched_RSB | | | | Negative Control |
| Biotin- | Biotin-Enriched_rsb | 10% | 3 | Test condition and performance for |
| Enriched_RSB | | biotin-G | | Biotin Enrichment with an additional |
| | | | | rinse prior to DNA capture elution. |
| Biotin- | Biotin-Enriched_HEB | SOP | 1 | Biotin-Enriched_HEB |
| Enriched_HEB | | | | Negative Control |
| Biotin- | Biotin-Enriched_HEB | 0.625, | 3/dNTP | Biotin-G titration test conditions |
| Enriched_HEB | | 1.25, 2.5, | condition | for modified biotin enrichment |
| | | 5, 10% | | prep using hybridization |
| | | biotin-G | | enrichment buffer |
| Biotin- | Biotin- | 10% | 1 | Biotin-Enriched_HEB using normal |
| Enriched_HEB | Enriched_HEB_regular_PCR | biotin-G | | indexing PCR instead of biotin |
| Standard PCR | | | | enrichment conditions. |
| reduced strand | Biotin- | 10% | 1 | Feasibility test for biotin enrichment |
| regen rxn | Enriched_HEB_low_regen_PCR | biotin-G | | condition using reduced strand |
| | | | | regeneration reaction volumes. |

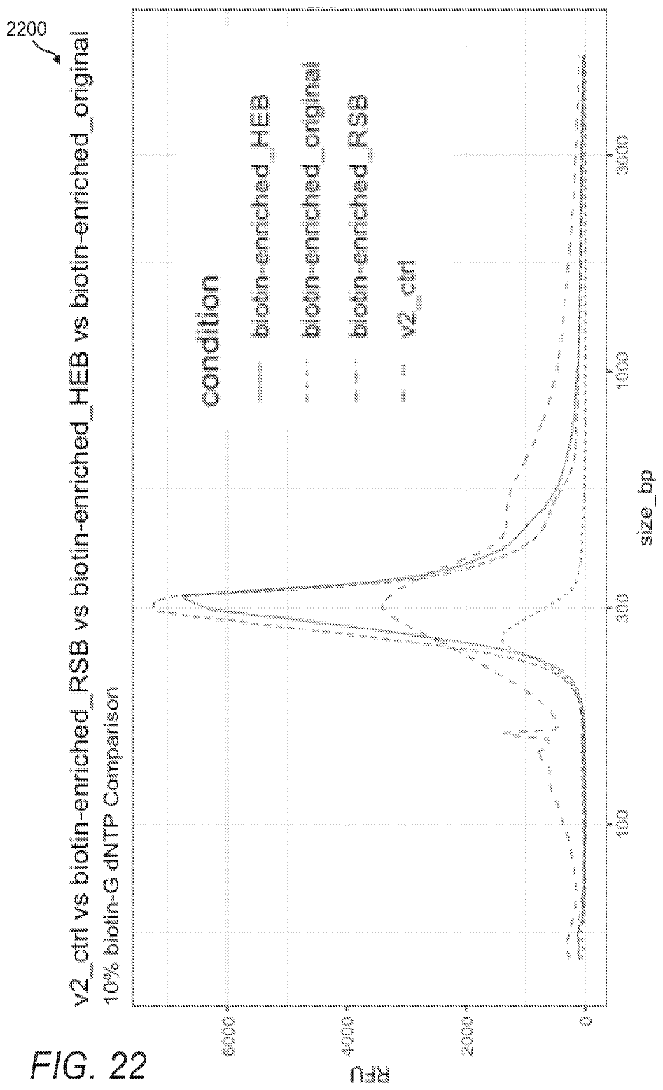

FIG. 22 is a plot 2200 showing the NGS Fragment Analyzer library profile comparison for V2 SOP, Biotin-Enriched_RSB, Biotin-Enriched_HEB, and Biotin-Enriched_original experimental conditions shown in Table 14. In this example, the data for conditions using 10% biotin-dGTP are shown. The data show that the original biotin enrichment method (Biotin-Enriched_original) has fragment sizes shorter than the V2 control (V2 SOP) and generates shorter libraries. Both the RSB rinse and HEB wash substitution modifications to the biotin enrichment process helped to recover larger fragments and generate library profiles more in line with and similar to V2 control libraries, albeit with a slightly narrower library distributions and lower amounts of dimers. In addition, both the RSB rinse and HEB wash replacement modifications yielded libraires that do not show a size shift to shorter fragments. Based on the relative proportion of the peak heights, which is a rough estimate of the yields, we observed that the yields are 4-5 times higher than the original condition with no modifications. Library yields for the different conditions tested using 10% biotin-dGTP (biotin-G) are summarized in Table 15.

TABLE 15
Library yields for V2 control, Biotin-Enriched
RSB, Biotin-Enriched HEB, and Biotin-Enriched
original libraries using 10% biotin-dGTP.

| | Dilution | Mean | Mean |
|---|---|---|---|
| Condition | Factor | (ng/μL) | Yield (ng) |
| Biotin-Enriched_HEB | 10.0 | 5.2 | 1288.9 |
| Biotin-Enriched_original | 10.0 | 1.0 | 258.4 |
| Biotin-Enriched_RSB | 10.0 | 4.7 | 1178.1 |
| V2_ctrl | 100.0 | 5.4 | 13396.3 |

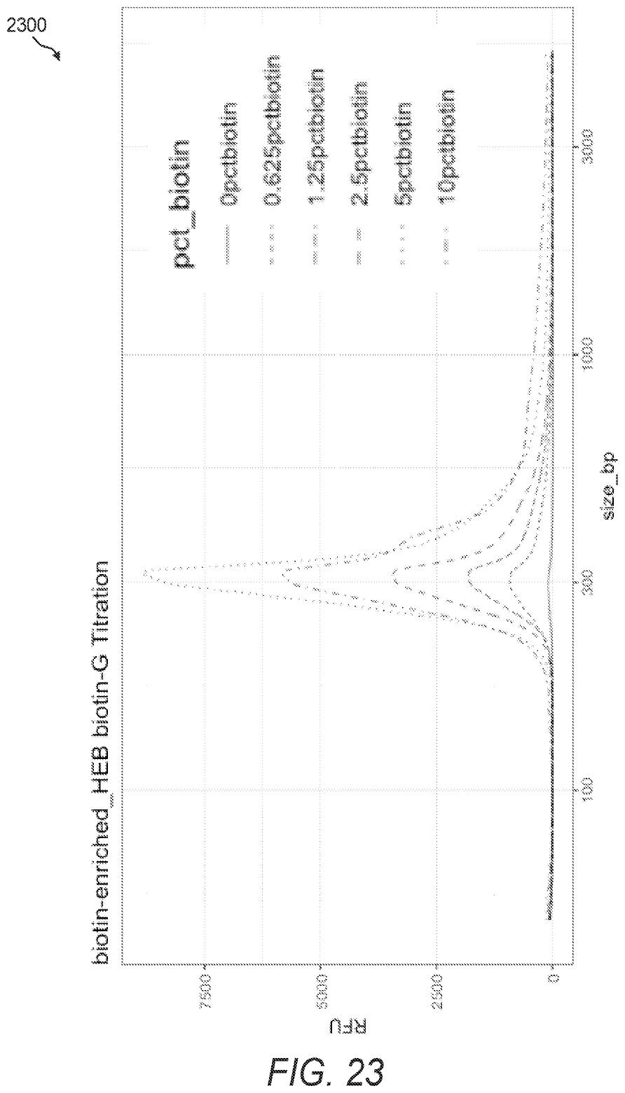

FIG. 23 is a plot 2300 showing Biotin-Enriched_HEB library profiles by percentage of biotin-dGTP used in the library preparation protocol. Libraries from the various biotin-dGTP titrations for the HEB wash conditions generated libraries with similar library profiles. However, the yields were proportionate and dependent on the percentage of biotin-dGTP used as shown in Table 16.

TABLE 16
Biotin-Enriched HEB biotin-dGTP titration
library quality control (QC) summary.

| | Percent | | Dilution | Mean | Mean |
|---|---|---|---|---|---|
| Condition | Biotin | | Factor | (ng/μL) | Yield (ng) |
| Biotin- | 0 | pctbiotin | 10.0 | 0.1 | 16.2 |
| Enriched_HEB | | | | | |
| Biotin- | 0.625 | pctbiotin | 10.0 | 0.9 | 235.9 |
| Enriched_HEB | | | | | |
| Biotin- | 1.25 | pctbiotin | 10.0 | 1.7 | 437.0 |
| Enriched_HEB | | | | | |
| Biotin- | 2.5 | pctbiotin | 10.0 | 2.8 | 709.0 |
| Enriched_HEB | | | | | |
| Biotin- | 10 | pctbiotin | 10.0 | 5.2 | 1288.9 |
| Enriched_HEB | | | | | |
| Biotin- | 5 | pctbiotin | 10.0 | 5.9 | 1468.6 |
| Enriched_HEB | | | | | |

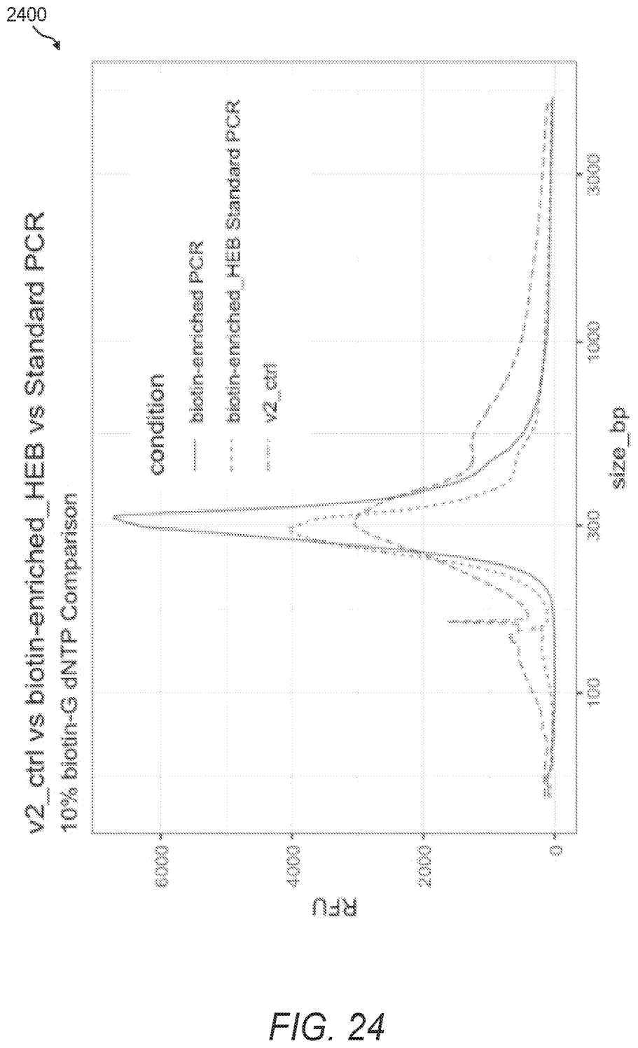

Replacing the 1×B+W buffer with the HEB buffer allows the use of standard V2 PCR conditions. FIG. 24 is a plot 2400 showing the library fragment size distributions for libraries prepared using the 1×B+W buffer (Biotin-Enriched_PCR) and the HEB buffer (Biotin-Enriched_HEB standard PCR) conditions using 10% biotin-dGTP. The standard V2 PCR conditions were used as a control. Standard PCR conditions use a higher primer concentration and more PCR cycles, which allows for high yields. The data show that biotin enriched libraries generated using HEB buffer and standard PCR conditions have library distributions similar to libraries that were generated using the 1×B+W buffer. However, the yields for libraries generated using the HEB buffer (Biotin-Enriched_HEB standard PCR) are higher compared to the Biotin-Enriched PCR condition as shown in Table 17.

TABLE 17
FA quantification summary for Biotin-Enriched PCR
vs Biotin-Enriched_HEB standard PCR conditions.

| | Dilution | Mean | Mean |
|---|---|---|---|
| Condition | Factor | (ng/μL) | Yield (ng) |
| Biotin-Enriched PCR | 10.0 | 5.2 | 1288.9 |
| Biotin-Enriched_HEB | 100.0 | 3.1 | 7672.5 |
| Standard PCR | | | |
| V2_ctrl | 100.0 | 6.1 | 15312.0 |

Recovery of longer fragments and higher yields in library output using the biotin enrichment library preparation protocol are improved by changing the wash buffer from a high salt buffer (i.e., 1×B+W buffer) to a lower salt buffer (e.g., hybridization enrichment buffer (HEB)). In some cases, an additional RSB rinse step after washing with the 1×B+W may also be used to provide both longer fragment recovery and higher yields. However, replacing the 1×B+W buffer with the hybridization enrichment buffer (HEB) may be more operationally and/or automation conducive and allows for the use of standard PCR conditions.

### 6.10.3. Optimization of Biotin Labeling

The POC experiment tested a broad range of biotin-dGTP (0, 10, 33, and 100%) dNTP mixtures and we determined that the 10% condition (10% biotin-dGTP in the dNTP mix) provided the best overall performance. To further evaluate the percentage of biotin-dGTP to use in the biotin enrichment library process, we designed an experiment to determine the percentage of biotin-dGTP in the linear amplification dNTP mix that balances and maintains high specificity for hypermethylated fragments and molecular recovery (i.e., conversion efficiency). For this experiment, we used 12.5 ng of PC2 as the starting material for V2 automated bisulfite conversion and prepared libraries as detailed and described in Table 18. EDTA, which chelates magnesium, was added to the reaction buffer to prevent further polymerase or exonuclease activity from the linear amplification polymerase after nucleotide incorporation. Sequencing data were analyzed for various library metrics.

TABLE 18
Optimization of biotin labeling and control library conditions.

| | Library Prep | dNTP | No. of | |
|---|---|---|---|---|
| Library Condition | Method | Mix | Samples | Purpose |
| V2_control | V2 GMS | SOP | 3 | V2 GMS Baseline Control |
| V2_control | V2 GMS | 10% | 1 | V2 GMS Baseline Control |
| | | biotin-G | | using 10% biotin-G mix |
| Biotin- | Biotin- | SOP | 1 | Biotin-Enriched_EDTA |
| Enriched_EDTA_negative | Enriched_EDTA | | | Negative Control |
| Biotin- | Biotin- | 0.625, 1.25, | 3/dNTP | Biotin-G titration test |
| Enriched_EDTA_dNTPpct | Enriched_EDTA | 2.5, 5, 10% | condition | conditions for modified |
| | | biotin-G | | Biotin-Enriched_EDTA |
| | | | | prep using hybridization |
| | | | | enrichment buffers |

Each library was evaluated using the NGS Fragment Analyzer, enriched using single plex V2 automated target hybridization enrichment with a subset of the Compass enrichment panel, sequenced to a target depth of 25M reads (˜168 samples/S2 Novaseq FC), and the data analyzed using methyl_3.18.0-TMv3_Doppler_custom pipeline analysis with reads subsampled to 20M. The subset enrichment panel should provide similar classification performance to the Compass panel. The smaller panel size was used to test coverage gains from smaller panels sizes in proof-of-concept testing.

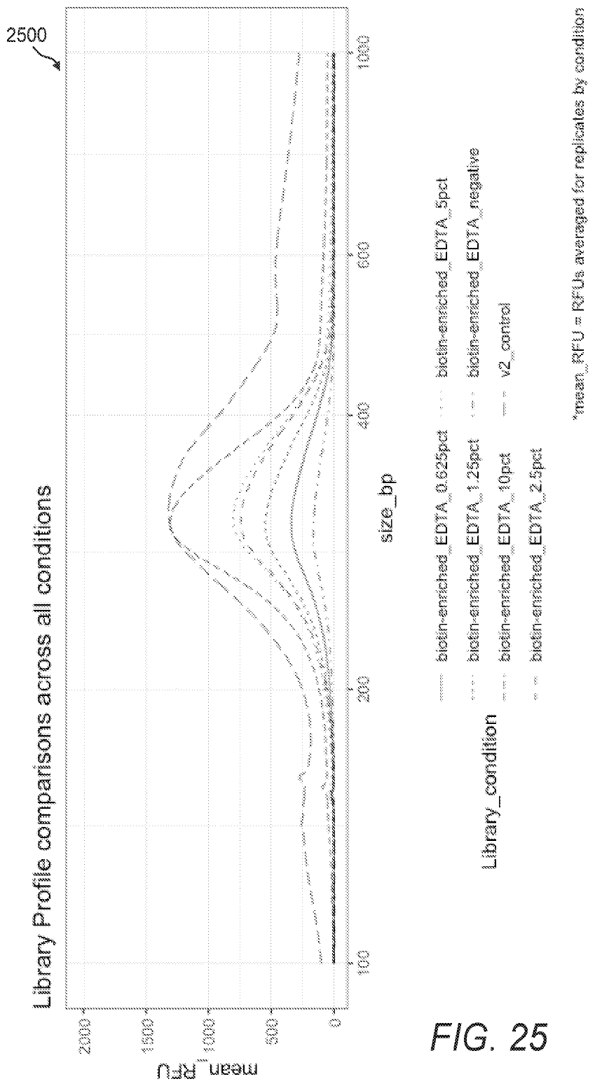

FIG. 25 is a plot 2500 showing the Fragment Analyzer traces for the library profile comparisons across all biotin-dGTP labeling and V2 control condition described in Table 18. The data show that libraries were generated for a range of biotin-dGTP percentages, from as low as 0.625% to 10% (“Biotin-Enriched_EDTA). All the biotin libraries (”Biotin-Enriched_EDTA) have similar library profiles, which are comparable to V2 control libraries, albeit slightly narrower. Library yields are shown in Table 19. Library yields are dependent on the percentages of biotin-dGTP used in the dNTP mix as higher percentages led to higher yields.

TABLE 19
Summary of Fragment Analyzer quantitation for biotin labeling optimization.

| Library_condition | mean_size | mean_ng_μL | Yield_ng | fold_above_bkgrd |
|---|---|---|---|---|
| V2_control | 502.8 | 594.4 | 14859.1 | 13.8 |
| Biotin- | 377.7 | 346.3 | 8658.7 | 8 |
| Enriched_EDTA_10 pct | | | | |
| Biotin- | 348 | 253.9 | 6346.7 | 5.9 |
| Enriched_EDTA_5 pct | | | | |
| Biotin- | 337.3 | 193.4 | 4834.4 | 4.5 |
| Enriched_EDTA_2.5 pct | | | | |
| Biotin- | 331.7 | 140.3 | 3508.6 | 3.3 |
| Enriched_EDTA_1.25 pct | | | | |
| Biotin- | 327.3 | 85 | 2125.6 | 2 |
| Enriched_EDTA_0.625 pct | | | | |
| Biotin- | 312 | 43 | 1076.2 | 1 |
| Enriched_EDTA_negative | | | | |

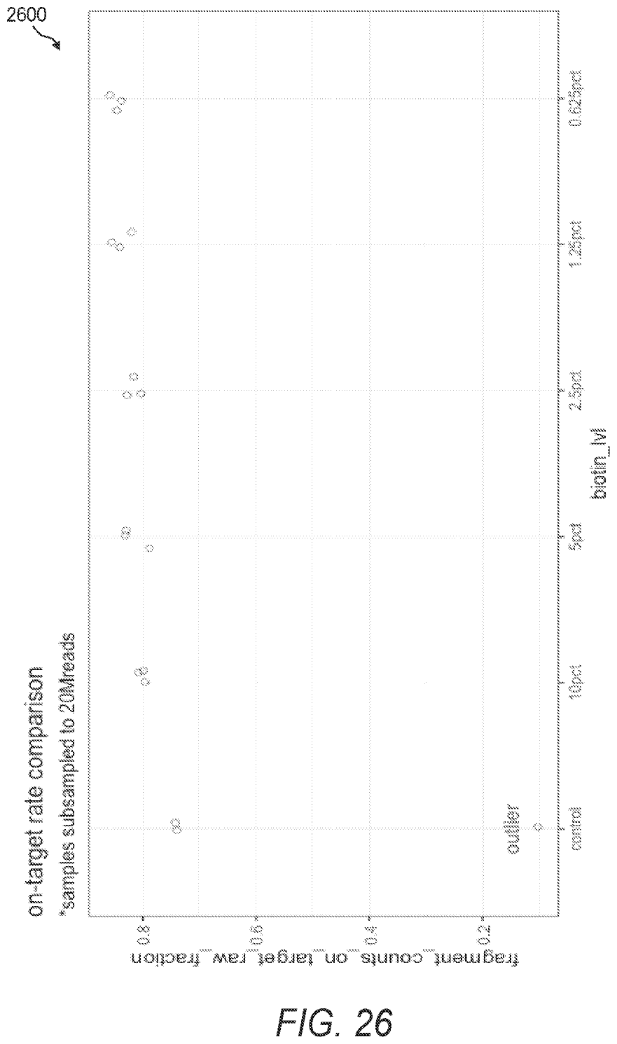

Analyzing the on-target rates for each library, we observed that one of the V2 control libraries had an extremely low and unexpected on-target rate, which is indicative of library target enrichment failure. Therefore, this data point was removed from subsequent analysis. FIG. 26 is a plot 2600 showing the comparison of on-target rates for the libraries in the biotin labeling optimization experiment in Table 18. Samples were subsampled to 20M reads. Sequencing data was assessed using the fragment_counts_on_target_raw_fraction metric. A summary of the on-target rates for the different libraries is shown in Table 20.

TABLE 20
Summary of on-target rates for the biotin optimization
experiment with outlier point included.

| | | |
|---|---|---|
| Library Prep | Biotin Level | Mean ± SD |

| | | | |
|---|---|---|---|
| Biotin-Enriched EDTA | 10 | pct | 0.801 ± 0.006 |
| Biotin-Enriched EDTA | 5 | pct | 0.817 ± 0.024 |
| Biotin-Enriched EDTA | 2.5 | pct | 0.816 ± 0.012 |
| Biotin-Enriched EDTA | 1.25 | pct | 0.838 ± 0.018 |
| Biotin-Enriched EDTA | 0.625 | pct | 0.848 ± 0.011 |

| | | |
|---|---|---|
| V2 | control | 0.528 ± 0.368 |

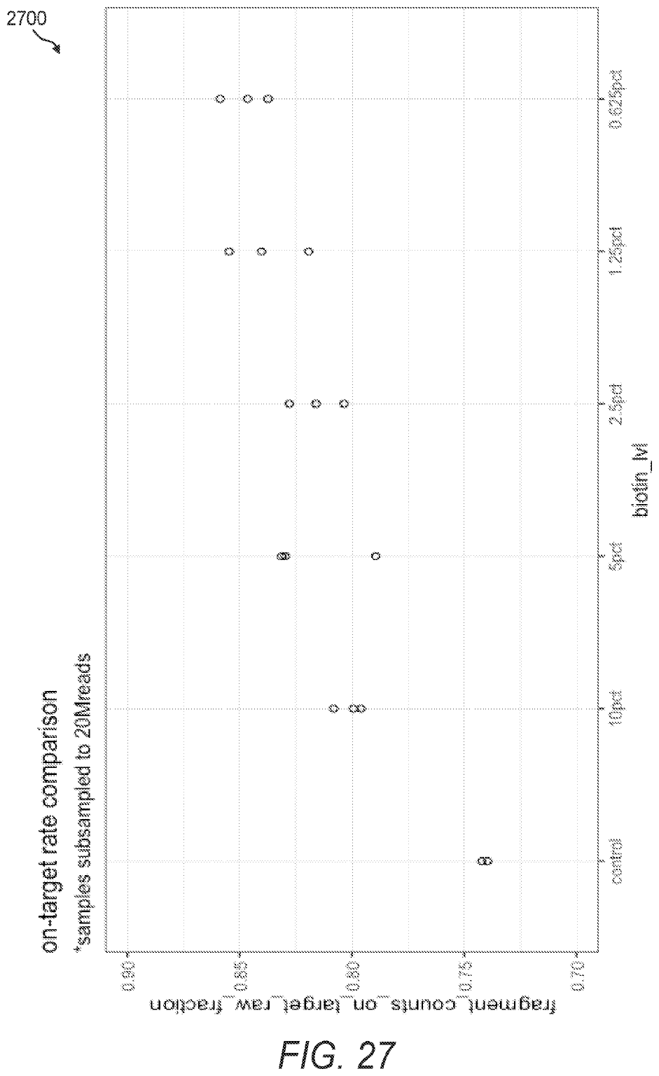

After removing the V2 control outlier data point (as noted above), the on-target rates for the biotin enriched libraries were slightly higher than the than the on-target rates for the V2 controls. FIG. 27 is a plot 2700 showing the on-target rates for the different libraries in the biotin labeling optimization experiment with the V2 control outlier point removed. A summary of the on-target rates with the V2 control outlier removed is shown in Table 21. The data show that the V2 control libraries have an on-target rate of 75% whereas biotin enriched libraries have on-target rates in the range of 80% to 85%, with decreasing biotin percentages appearing to lead to higher on-target rates.

TABLE 21
Summary of on-target rates for the biotin labeling
optimization experiment with outlier removed.

| | | |
|---|---|---|
| Library Prep | Biotin Level | Mean ± SD |

| | | | |
|---|---|---|---|
| Biotin-Enriched EDTA | 10 | pct | 0.801 ± 0.006 |
| Biotin-Enriched EDTA | 5 | pct | 0.817 ± 0.024 |
| Biotin-Enriched EDTA | 2.5 | pct | 0.816 ± 0.012 |
| Biotin-Enriched EDTA | 1.25 | pct | 0.838 ± 0.018 |
| Biotin-Enriched EDTA | 0.625 | pct | 0.848 ± 0.011 |

| | | |
|---|---|---|
| V2 | control | 0.741 ± 0.002 |

We next compared the abnormal coverage of hypermethylated and hypomethylated fragments (linear_filtered_abnormal_coverage_hyper_cpg_means) in the biotin enriched and V2 control libraries. Abnormal fragments may be hypermethylated fragments that are indicative of a disease state such as cancer. Hypomethylated fragments and/or unmethylated fragments may be indicative of a “normal” state relative to a cancer state.

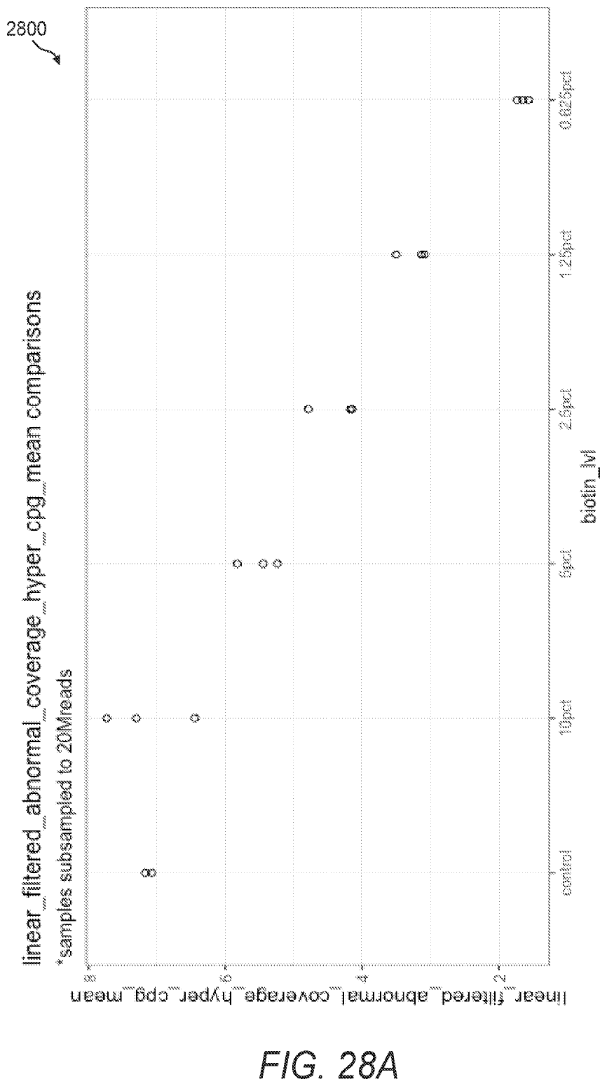

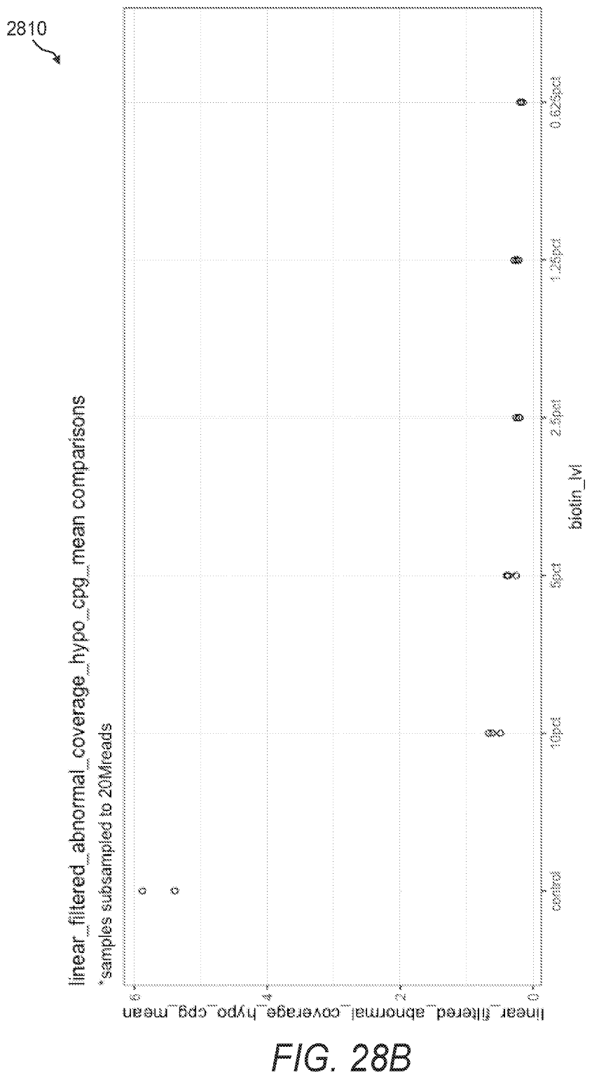

FIG. 28A is a plot 2800 showing the abnormal coverage of hypermethylated fragments in biotin enriched and V2 control libraries described in Table 18. A summary of the abnormal coverage of hypermethylated fragments is shown in Table 22. At 10% biotin-dGTP, abnormal coverage of hypermethylated fragments was similar to the abnormal coverage in the V2 control library. Decreasing the biotin-dGTP percentage below 10% resulted in a reduction in the hypermethylated coverage, which indicates that molecules may be lost from the assay.

FIG. 28B is a plot 2810 showing the abnormal coverage of hypomethylated fragments in biotin enriched and V2 control libraries described in Table 18. At 10% biotin-dGTP, abnormal coverage of hypomethylated fragments is low indicating that hypomethylated fragments are depleted in the biotin enrichment process.

Referring now to FIG. 28A, FIG. 28B, and Table 22, we concluded that 10% biotin-dGTP is the optimal percentage for achieving both high enrichment for methylated abnormal fragments, which are on par with V2 controls while still depleting non-methylated or hypomethylated fragments.

TABLE 22
Summary table for abnormal coverage
for hypermethylated fragments.

| | | |
|---|---|---|
| Library Prep | Biotin Level | Mean ± SD |

| | | | |
|---|---|---|---|
| Biotin-Enriched EDTA | 10 | pct | 7.16 ± 0.66 |
| Biotin-Enriched EDTA | 5 | pct | 5.5 ± 0.3 |
| Biotin-Enriched EDTA | 2.5 | pct | 4.37 ± 0.36 |
| Biotin-Enriched EDTA | 1.25 | pct | 3.24 ± 0.23 |
| Biotin-Enriched EDTA | 0.625 | pct | 1.65 ± 0.08 |

| | | |
|---|---|---|
| V2 | control | 7.12 ± 0.07 |

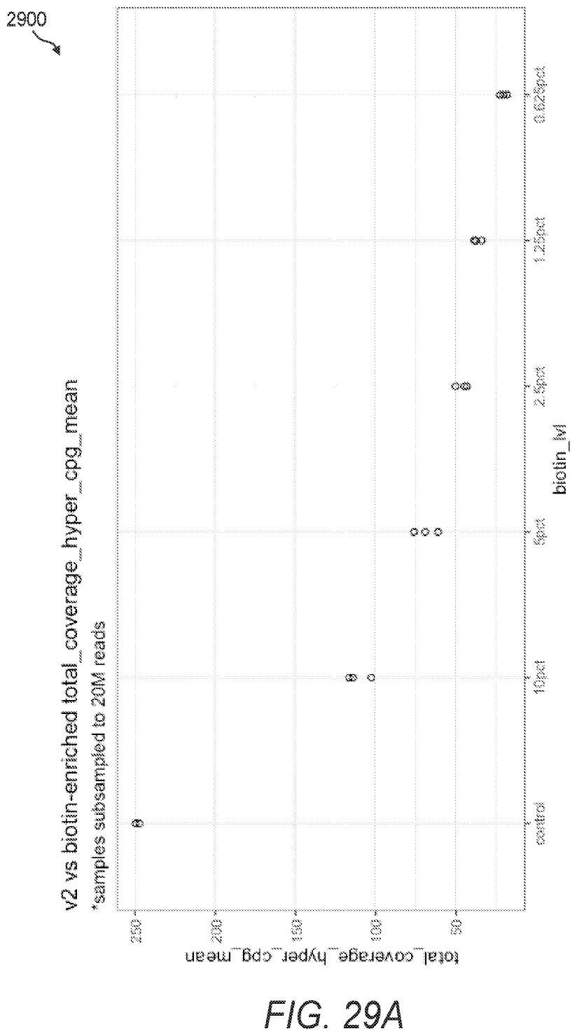

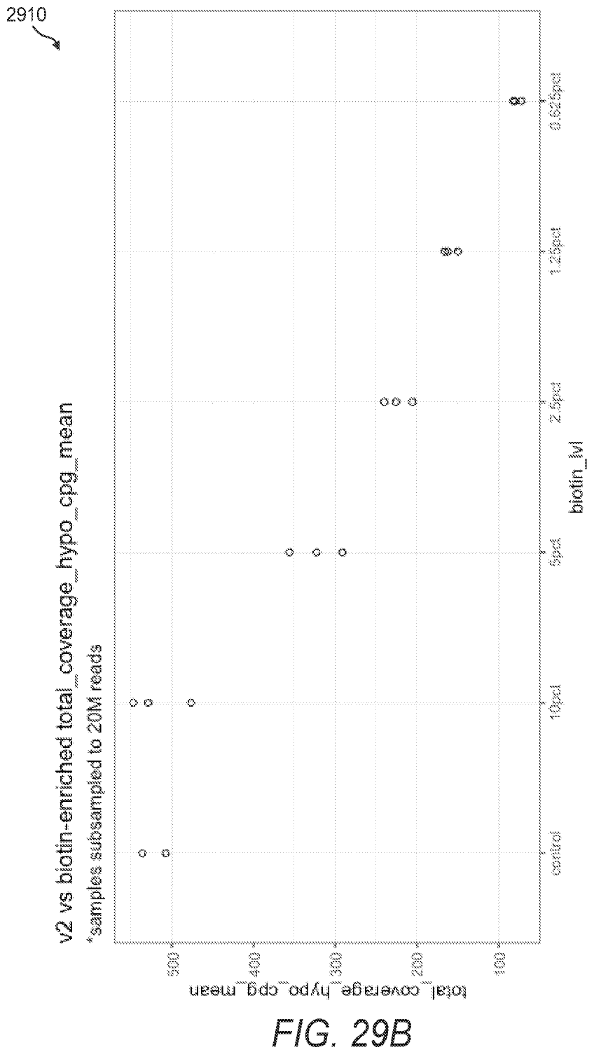

We also compared the total coverage of fragments in the biotin enriched and V2 control libraries. FIG. 29A is a plot 2900 showing the total coverage of hypermethylated fragments (total_coverage_hyper_cpg_means) in the biotin enriched and V2 control libraries described in Table 18. The data show the total coverage of the “targets” considered to be hypermethylated in cancer. Previously (see FIG. 28A and Table 22), we demonstrated equal to better performance on “abnormal hyper coverage”. That there is lower total coverage in the biotin enriched libraries therefore means that a greater fraction of the fragments in the library were hypermethylated and the library is more informative from the removal of the “healthy” fragments that have low methylation.

FIG. 29B is a plot 2910 showing the total coverage of hypomethylated fragments (total_coverage_hypo_cpg_means) in the biotin enriched and V2 control libraries. For the hypomethylated coverage, the “healthy” state is methylated, and the cancer state is hypomethylated. The data show that we retained the healthy methylated fragments but had excluded the abnormal hypomethylated fragments.

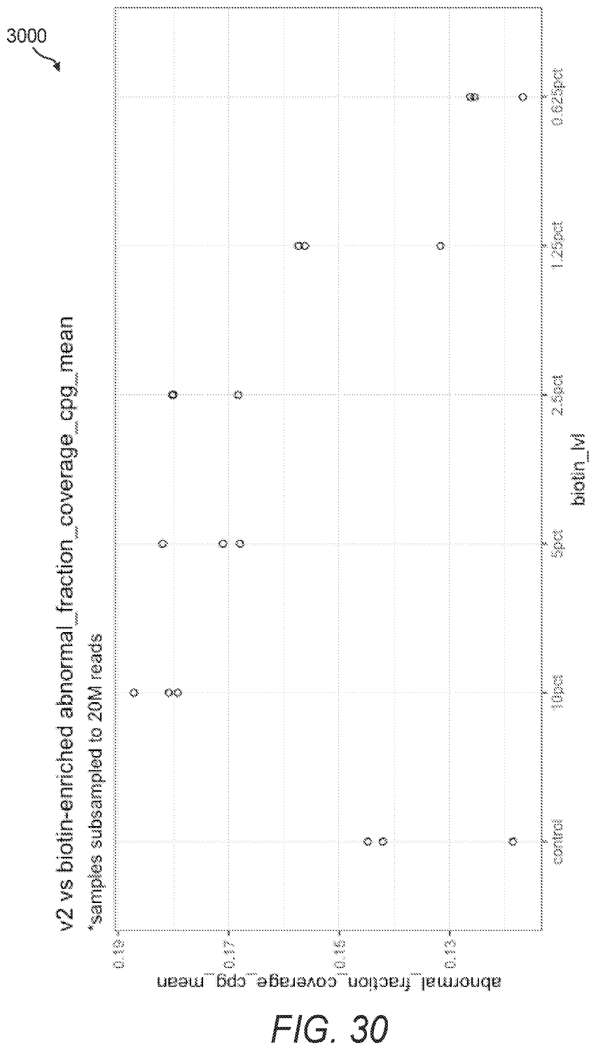

Additionally, the abnormal fraction coverage (abnormal_fraction_coverage_cpg_mean) for the various biotin titrations is equivalent or better than that of the V2 control libraries. FIG. 30 is a plot 3000 showing the abnormal fraction CpG coverage for biotin enriched and V2 control libraries described in Table 18. A summary of the abnormal fraction CpG coverage metric is shown in Table 23. The data show that increasing biotin levels or percentages led to increased abnormal fractions with the 10% biotin condition obtaining the best performance.

TABLE 23
Summary table for abnormal fraction CpG coverage metric.

| | | |
|---|---|---|
| Library Prep | Biotin Level | Mean ± SD |

| | | | |
|---|---|---|---|
| Biotin-Enriched EDTA | 10 | pct | 0.182 ± 0.004 |
| Biotin-Enriched EDTA | 5 | pct | 0.174 ± 0.007 |
| Biotin-Enriched EDTA | 2.5 | pct | 0.176 ± 0.007 |
| Biotin-Enriched EDTA | 1.25 | pct | 0.148 ± 0.014 |
| Biotin-Enriched EDTA | 0.625 | pct | 0.123 ± 0.005 |

| | | |
|---|---|---|
| V2 | control | 0.135 ± 0.014 |

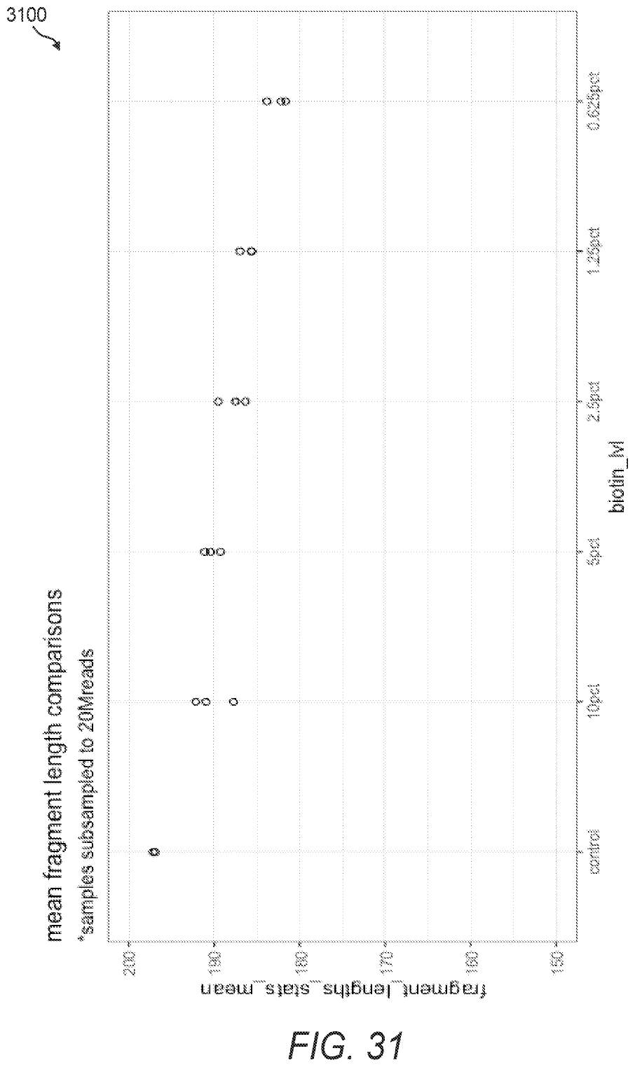

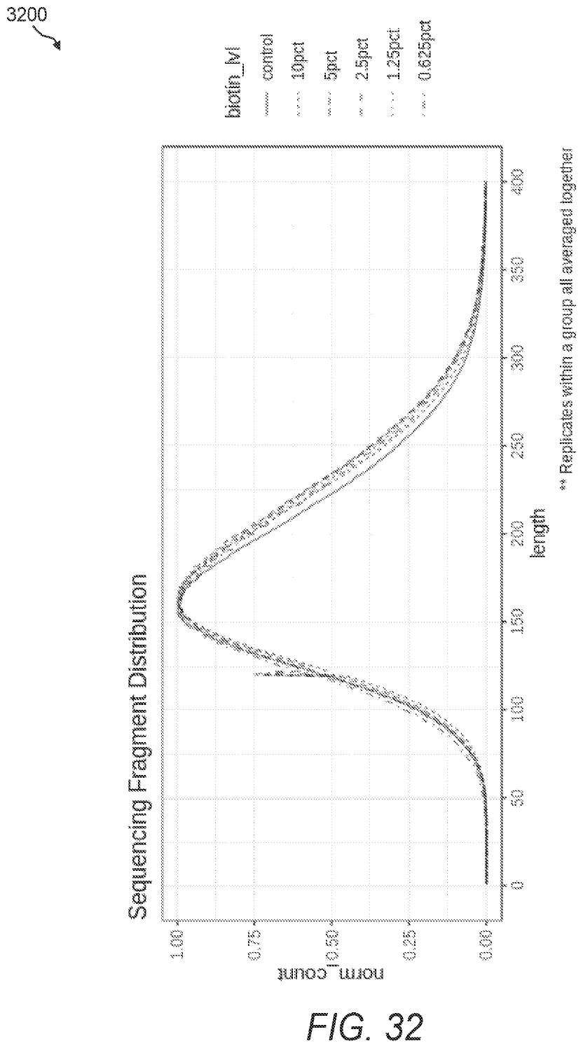

FIG. 31 is a plot 3100 showing a comparison of sequencing fragment lengths in biotin enriched and V2 control libraries prepared using different percentages of biotin-dGTP described in Table 18. A summary of the fragment size data is shown in Table 24. FIG. 32 is a plot 3200 showing the sequencing fragment distributions in biotin enriched and V2 control libraries prepared using different percentages of biotin-dGTP described in Table 18.

Referring now to FIG. 31, FIG. 32, and Table 24, the data show that the biotin enriched libraries are shorter than V2 control libraries by a few bases, but the difference is not very substantial since when comparing the sequencing fragment distribution for the various library conditions (see FIG. 32), we observed that the profiles are nearly identical and overlap well. A spike in the sequencing fragment distributions was observed at 120 bases for all libraries. This peak corresponds to probe contamination, which was likely due to the use of a contaminated reagent.

TABLE 24
Sequencing fragment lengths for the biotin
enriched and V2 control libraries.

| | | |
|---|---|---|
| Library Prep | Biotin Level | Mean ± SD |

| | | | |
|---|---|---|---|
| Biotin-Enriched EDTA | 10 | pct | 190.3 ± 2.28 |
| Biotin-Enriched EDTA | 5 | pct | 190.28 ± 0.94 |
| Biotin-Enriched EDTA | 2.5 | pct | 187.79 ± 1.61 |
| Biotin-Enriched EDTA | 1.25 | pct | 186.09 ± 0.78 |
| Biotin-Enriched EDTA | 0.625 | pct | 182.56 ± 1.15 |

| | | |
|---|---|---|
| V2 | control | 197.03 ± 0.16 |

In a biotin enriched library, uninformative fragments (i.e., hypomethylated relative to a targeted hypermethylation level) are essentially eliminated from the assay. Because the uninformative fragments have been eliminated, a lower sequencing depth may be used to achieve the same coverage of hypermethylated targets.

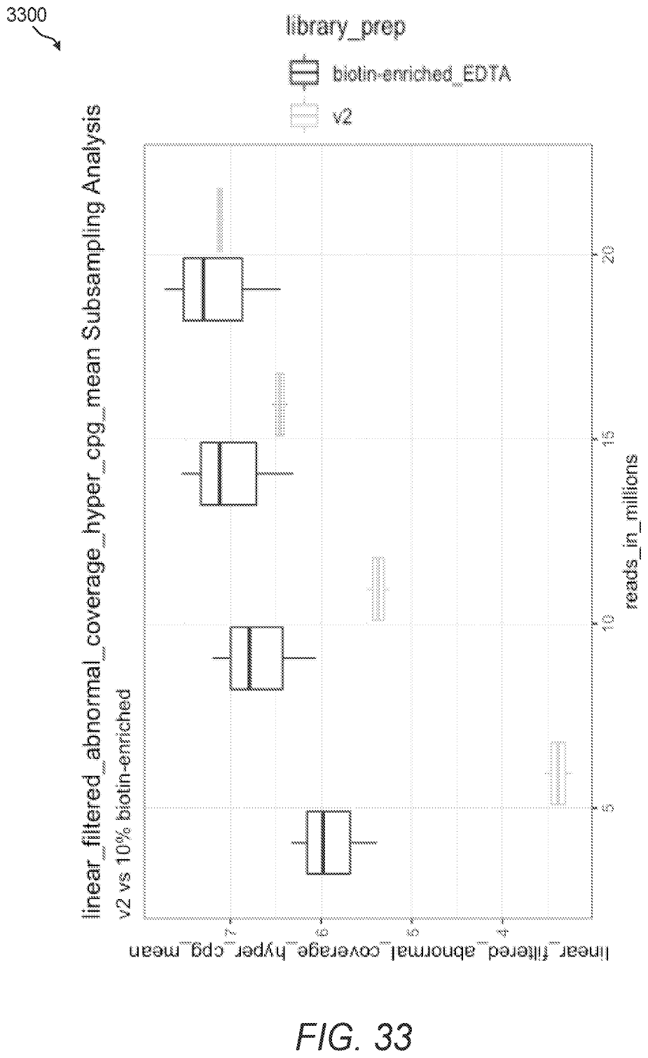

FIG. 33 is a plot 3300 showing the abnormal coverage of hypermethylated fragments in biotin enriched and V2 control libraries at lower sequencing depths. In this example, the biotin enriched library was prepared using 10% biotin-dGTP. The data show that at lower sequencing depths ranging from 5 million reads to 20 million reads, coverage of hypermethylated fragments is equal to or greater in the biotin enriched library compared to the V2 control library (i.e., abnormal coverage saturates faster in the biotin enriched library).

### 6.10.4. Hybridization Study

The standard V2 library preparation protocol uses two rounds of hybridization enrichment to enrich for target sequences of interest. To determine the feasibility of using the biotin enrichment process with a single round of target hybridization enrichment, we generated biotin enriched libraries using either one or two rounds of hybridization enrichment. The standard V2 BSC library preparation protocol was used as a control method. An enrichment probe panel (referred to as Deflector panel) targeting only hypermethylated sequences was used for hybridization enrichment. The libraries were prepared, sequenced by NGS and various metrics of interest were used to evaluate the libraries.

Reagents specific to the streptavidin bisulfite ligand methylation enrichment protocol were Biotin-16-7-Deaza-7-Propargylamino-2′-deoxyguanosine-5′-Triphosphate (Biotin-dGTP) (available from TriLink; part number N-5010), dNTP set (available from ThermoFisher, part number 10297018), Strand Regeneration Primer (5′-ACACGACGCTCTTCCGATCT-3′) (IDT Custom), 5× VeraSeq ULtra DNA Polymerase (Qiagen, P7520L), and 5× VeraSeq Buffer II (Qiagen, B7102).

Software used in the study included Pipeline Data Analysis (software version: methyl_3.18.2-TMv3_Deflector_custom) and RStudio (software version: 3.6.1).

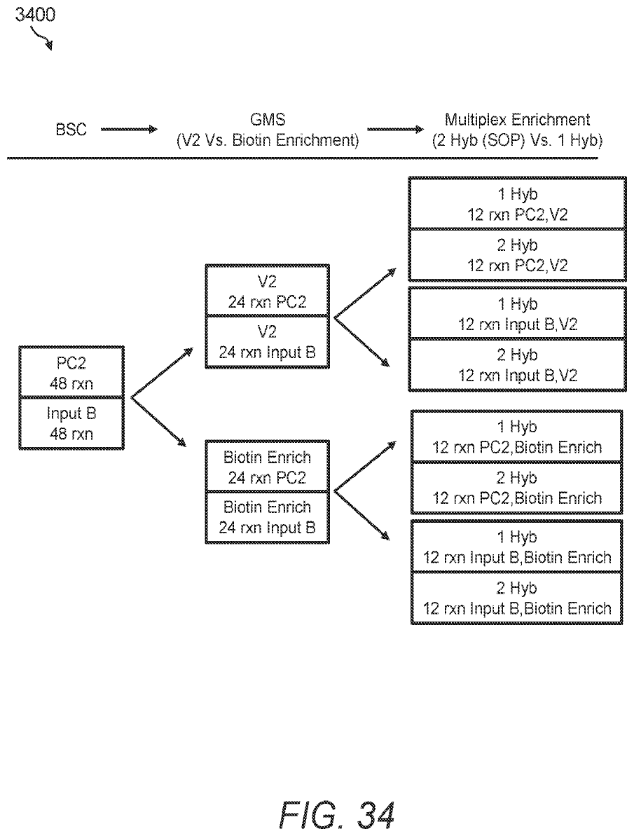

FIG. 34 illustrates a schematic diagram 3400 of experimental conditions and workflow for the target hybridization enrichment study. The experimental study included eight conditions: (i) two different input samples (i.e., Input B and PC2); (ii) two methylation sequencing library protocols, the standard V2 BSC library preparation protocol and the biotin enrichment library preparation protocol; and (iii) two target enrichment hybridization conditions, one round of hybridization enrichment (designated “1hyb”) or two rounds of hybridization enrichment (designated “2hyb” or “SOP”).

The experimental study used Input B and PC2 as sample inputs. Preparation of the Input B sample in a resuspension buffer (RSB) is described in Table 25. As shown in Table 25, the volume of sample prepared was for use in bisulfite conversion reactions performed in a half plate Labcyte 384-well plate.

TABLE 25
Recipe for preparation of Input B

| | Volume (μL) for |
|---|---|
| Reagent | half plate |
| Input B 1.25 ng/μL (G0315) | 600 |
| Resuspension Buffer | 720 |
| (RSB, 10 mM Tris, pH 8.0) | |
| Total Volume | 1320 |

Preparation of the PC2 sample in RSB is described in Table 26. As shown in Table 26, the volume of sample prepared was for use in bisulfite conversion reactions performed in a half plate Labcyte 384-well plate.

TABLE 26
Recipe for preparation of PC2

| | Volume (μL) for |
|---|---|
| Reagent | half plate |
| PC2 0.625 ng/μL (G0671) | 1200 |
| Resuspension Buffer | 160 |
| (RSB, 10 mM Tris, pH 8.0) | |
| Total Volume | 1360 |

The BSC reactions and library preparation protocols were performed in a series of multi-well microtiter plates. Briefly, aliquots of the prepared Input B and PC2 samples were manually pipetted into separate wells of a Labcyte 384-well plate for the BSC reactions. The BSC reactions were performed using the V2 library preparation automated protocol. Aliquots of the bisulfite converted PC2 (n=48), and Input B (n=48) samples were then transferred to separate wells of a 96-well microtiter plate for preparation of biotin enriched (PC2 n=24 and Input B n=24) and V2 control (PC2 n=24 and Input B n=24) libraries. The prepared libraries were then enriched using either one round of hybridization enrichment (n=12) or two rounds of hybridization enrichment (n=12).

The quality of the prepared biotin enriched and V2 control libraries was assessed using Fragment Analyzer (FA) quantitation and by AccuClear. All samples were sequenced together on a NovaSeq S2 flowcell. Various quality control (QC) metrics including bisulfite conversion ratio, on-target rate, LA filtered hyper abnormal coverage, total coverage hyper CpG mean, and abnormal fraction coverage were analyzed. Samples were analyzed with and subsampled to 1, 2.5, 5, 10, 15, 20, and 25M reads using the methyl_3.18.2-TMv3_Deflector_custom pipeline.

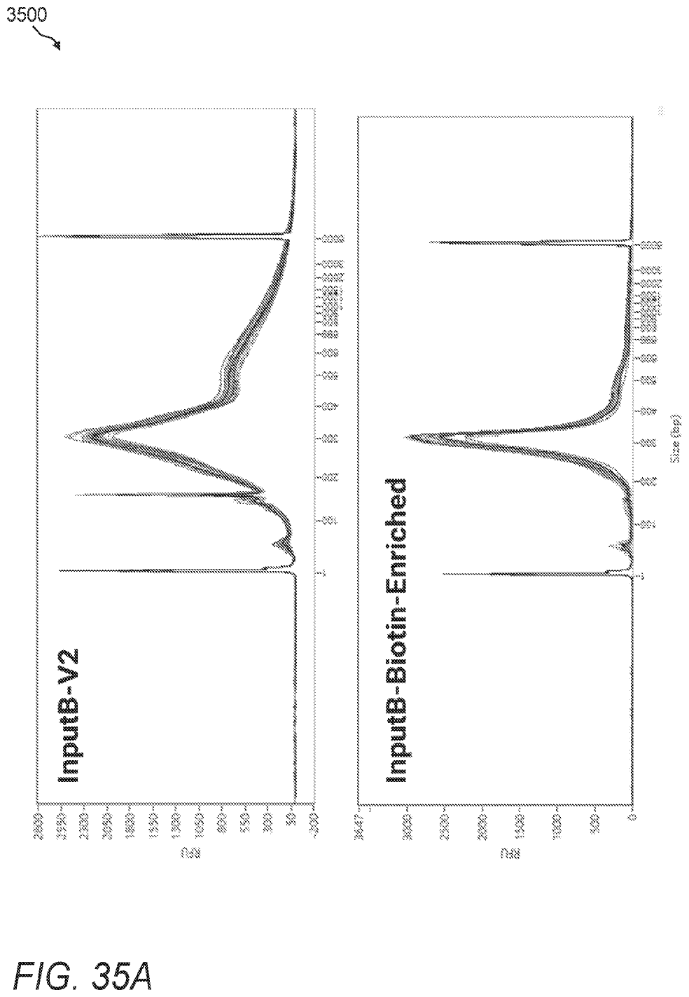

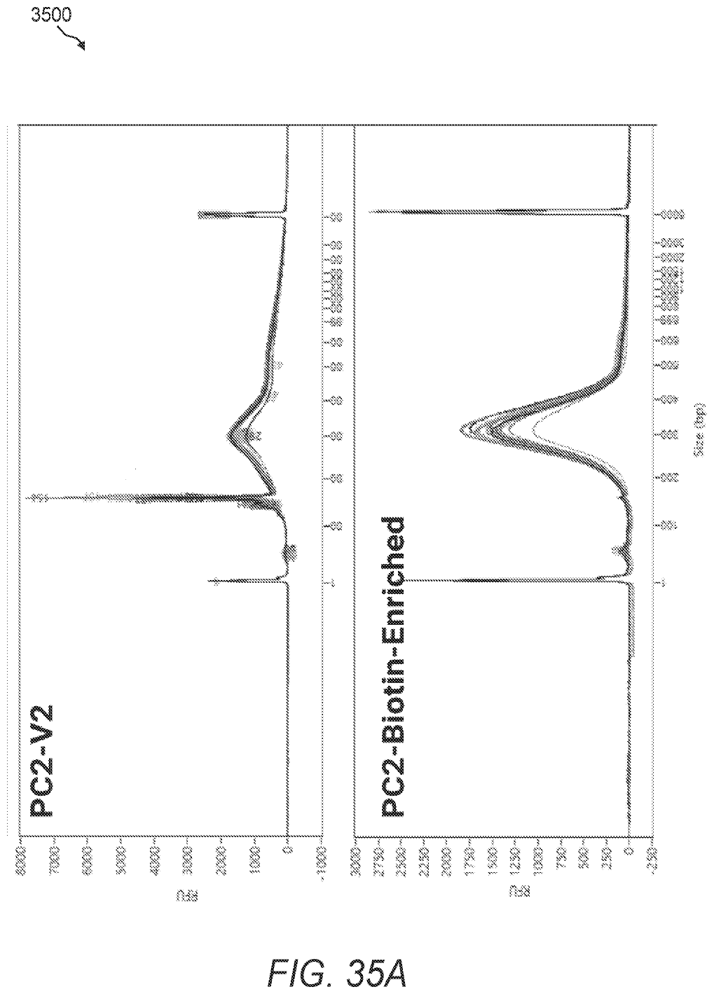

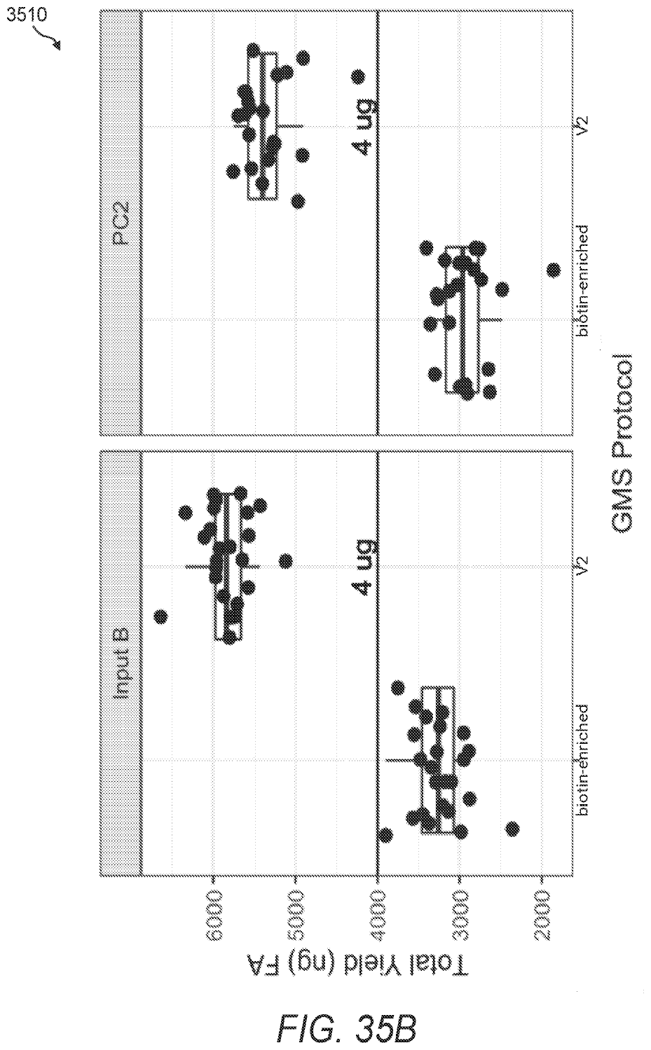

FIG. 35A is a panel of plots 3500 showing the Fragment Analyzer profiles for the PC2-V2, Input B-V2, PC2-biotin enriched (“PC2-Biotin-Enriched”), and Input B-biotin enriched (“Input B-Biotin-Enriched”) libraries in the hybridization enrichment study. The data show that the biotin enriched libraries have similar size distribution as V2 control libraries, except the biotin enriched libraries have much smaller primer dimer peaks and a narrower size distribution.

FIG. 35B is pair of plots 3510 showing the total yields by library preparation protocol for the Input B and PC2 libraries. The data show that the yields of biotin enriched libraries are 50% of V2 libraries. This result was expected because approximately half of input fragments are expected to be hypomethylated and therefor excluded from the biotin enriched libraries. The library yields are summarized in Table 27.

TABLE 27
Input B and PC2 library yields for
the hybridization enrichment study.

| | | Total |
|---|---|---|
| Library | Range | Yield (ng) |
| InputB_Biotin Enriched | 175 bp to 5000 bp | 3258.940 |
| InputB_V2 | 175 bp to 5000 bp | 5838.340 |
| PC2_Biotin Enriched | 175 bp to 5000 bp | 2962.995 |
| PC2_V2 | 175 bp to 5000 bp | 5399.280 |

In the examples that follow, libraries prepared using either the biotin enrichment or V2 control library preparation process and one round of hybridization enrichment are designated by “1Hyb”, and libraries prepared using two rounds of hybridization enrichment are designated by “SOP”.

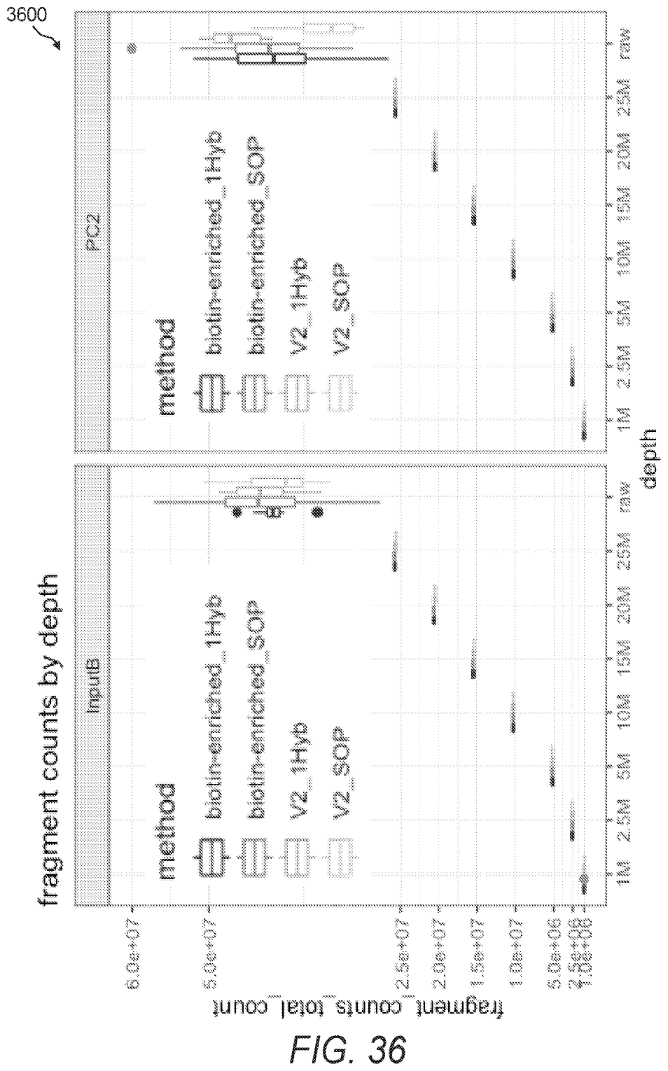

The hybridization enriched libraries were sequenced to a mean depth of 40M reads and samples were subsampled to various sequencing depths (raw, 25, 20, 15, 10, 5, 2.5, and 1M sequencing reads) to determine whether a lower sequencing depth could be used. FIG. 36 is a pair of plots 3600 showing fragment counts by sequencing depth for the Input B biotin enriched and V2 control libraries, and the PC2 biotin enriched and V2 control libraries. The data show that all libraries attained a minimum of 25M reads.

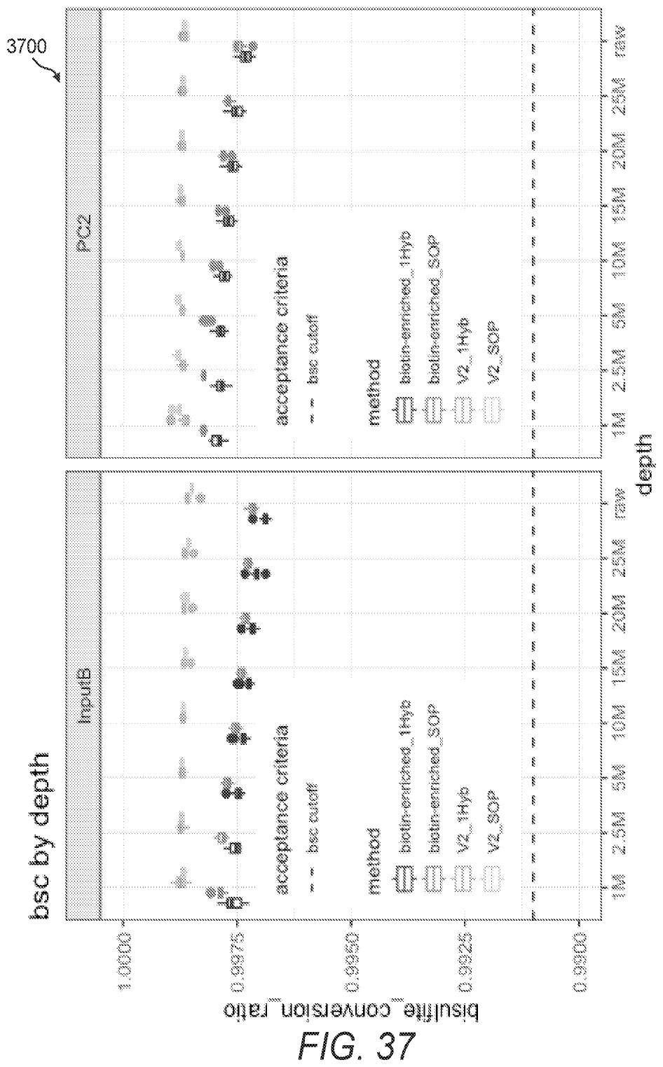

FIG. 37 is a plot 3700 showing the bisulfite conversion ratio by sequencing depth for the biotin enriched and V2 control Input B and PC2 libraries. The data show that there is an apparently lower bisulfite conversion efficiency in the biotin enriched libraries. The lower conversion efficiency observed may be due to an artifact of the bioinformatics process used in the analysis of the sequencing data (i.e., unconverted and/or partially converted fragments may be lost in the biotin enrichment process and therefore absent in the final library).

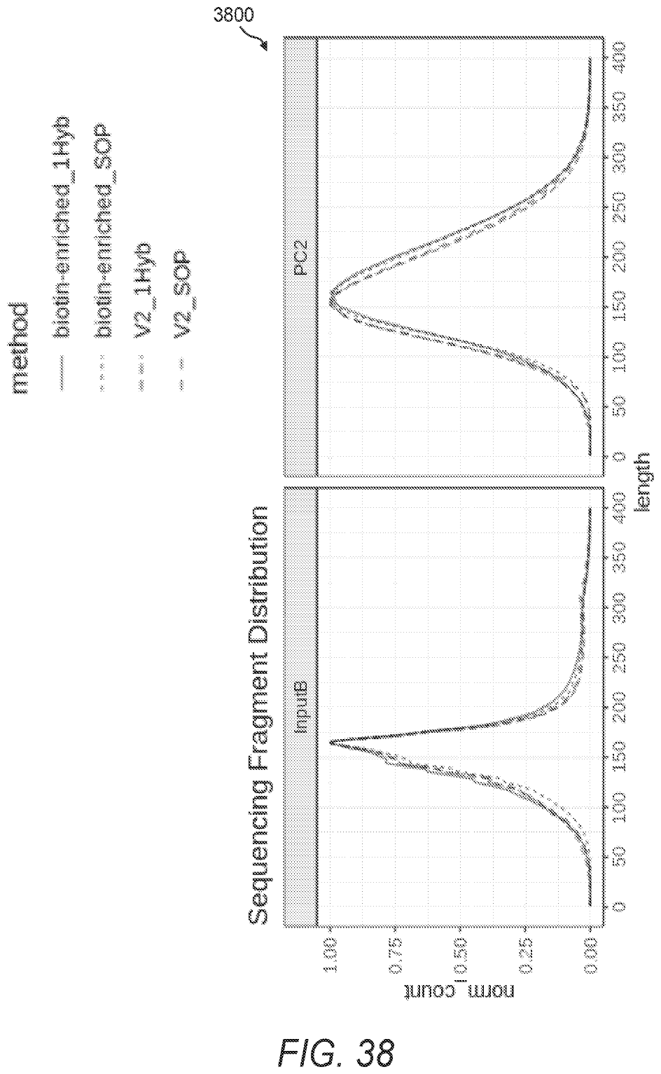

FIG. 38 is a plot 3800 showing sequencing fragment length distributions in the biotin enriched and V2 control libraries. The data show that biotin enriched libraries prepared using one or two rounds of hybridization enrichment have similar sequencing fragment length distributions to the V2 control library.

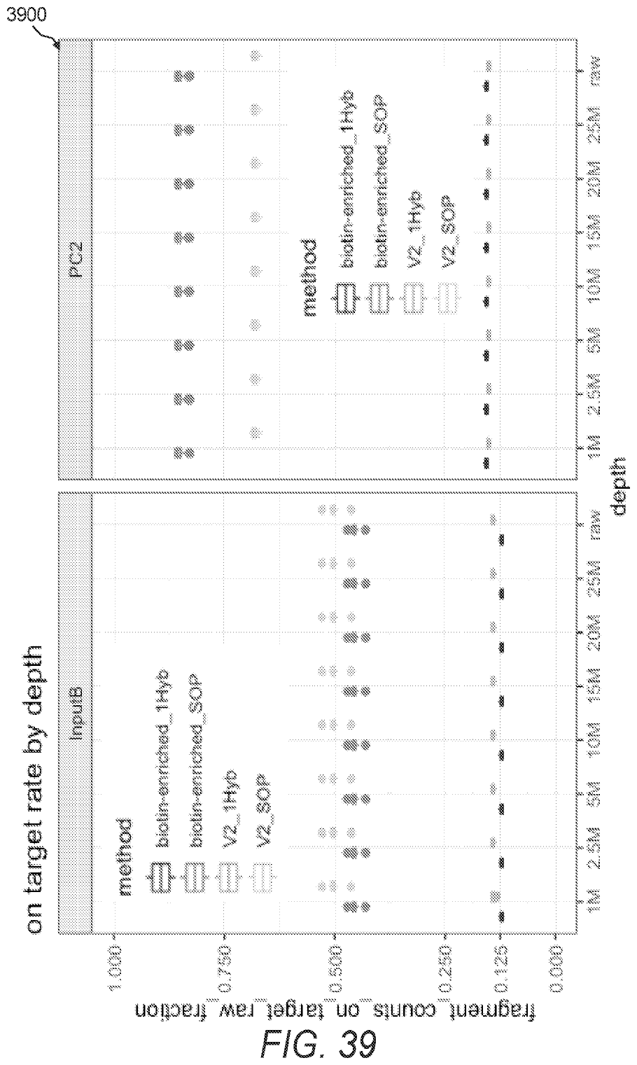

FIG. 39 is a pair of plots 3900 showing the on-target rate by depth comparison for the biotin enriched and V2 control libraries. The data show that both the biotin enriched and V2 control libraries prepared using one round of hybridization enrichment have a lower on-target rate at 12.5%, whereas the libraries prepared using two rounds of hybridization enrichment have a higher on-target rate of 75%.

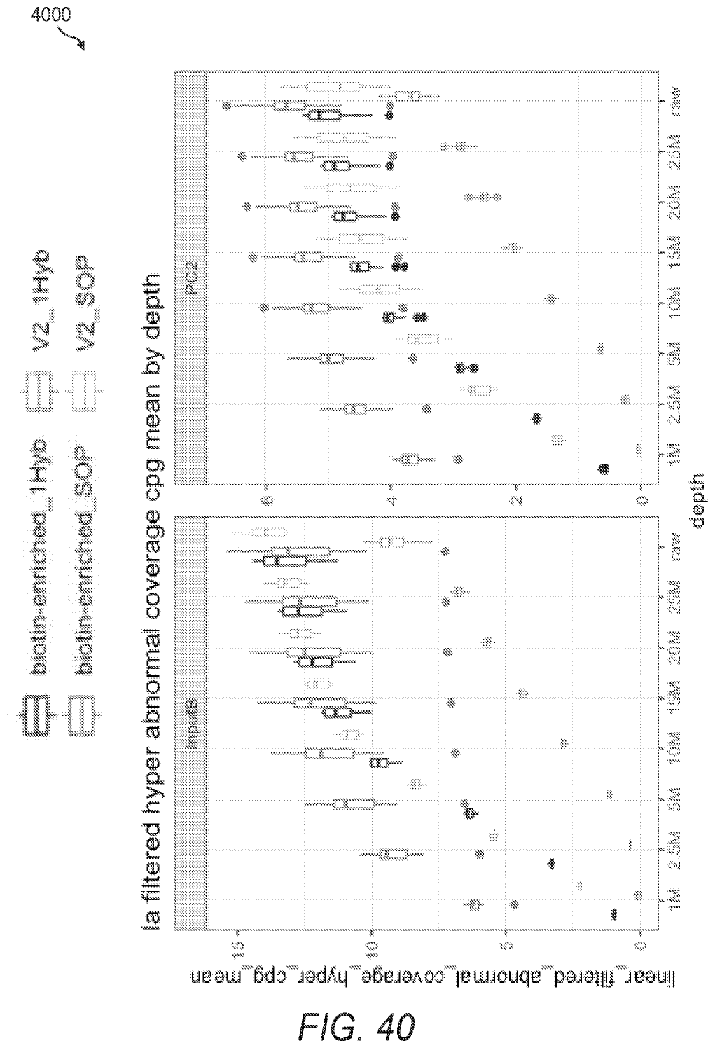

FIG. 40 is a pair of plots 4000 showing the abnormal coverage by depth for hypermethylated fragments (linear_filtered_abnormal_coverage-hyper_cpg_mean) in the biotin enriched and V2 control libraries. The data show that all biotin enriched libraries have higher hypermethylation abnormal coverage than V2 libraries prepared with 2 rounds of hybridization enrichment (V2 SOP) at less that 10M read depth. The biotin enriched libraries are saturated beyond 10M read depth. Because the biotin enriched libraries are saturated earlier, they would require less sequencing coverage than V2 SOP libraries.

The on-target rate of libraries prepared using one round of hybridization enrichment are lower than libraries prepared using two rounds of hybridization enrichment (see FIG. 39). However, because the uninformative fragments in the biotin enriched libraries have been removed biasing the libraries toward informative fragments, we see that at 10 to 15M reads we are achieving similar hypermethylated abnormal fragment coverage as observed in the standard V2 control libraries prepared using two rounds of hybridization enrichment.

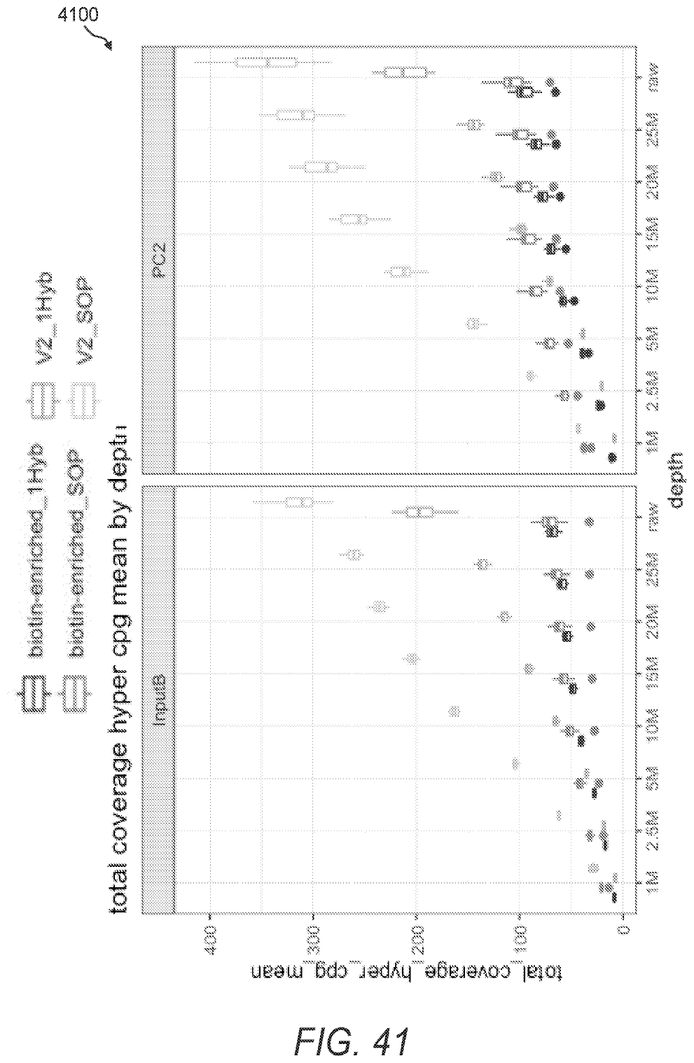

FIG. 41 is a pair of plots 4100 showing the total coverage by depth for hypermethylated fragments (total_coverage_hyper_cpg_mean) for the biotin enriched and V2 control libraries. The data show that biotin enriched libraries have lower mean total coverage per hypermethylated CpG than V2 control libraries, which is attributed to depletion of noninformative fragments in the biotin enriched libraries (i.e., the biotin enriched libraries are biased towards hypermethylated fragments). The data also show that biotin enriched libraries prepared using either one or two rounds of target hybridization enrichment have similar mean total coverage.

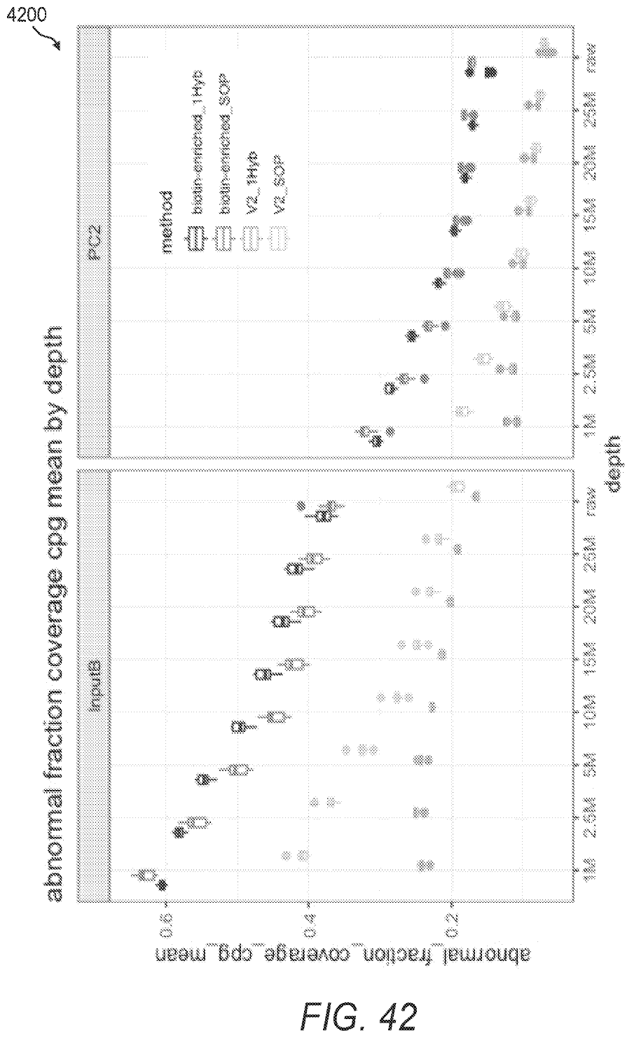

FIG. 42 is a pair of plots 4200 showing abnormal fraction coverage for the biotin enriched and V2 control libraries. The data show that biotin enriched libraries have a higher apparent abnormal fraction than V2 SOP control libraries, which is expected because the biotin enrichment protocol selects against “normal” or hypomethylated fragments.

It is to be understood that the figures and descriptions of the present disclosure have been simplified to illustrate elements that are relevant for a clear understanding of the present disclosure, while eliminating, for the purpose of clarity, many other elements found in a typical system. Those of ordinary skill in the art may recognize that other elements and/or steps are desirable and/or required in implementing the present disclosure. However, because such elements and steps are well known in the art, and because they do not facilitate a better understanding of the present disclosure, a discussion of such elements and steps is not provided herein. The disclosure herein is directed to all such variations and modifications to such elements and methods known to those skilled in the art.

Some portions of the above description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. The described operations and their associated modules may be embodied in software, firmware, hardware, or any combinations thereof.

The methods may be accomplished using robotics controlled by computers. The methods may be embodied in computer-readable instructions for controlling robotic operations to cause them to execute the disclosed methods.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment, thereby providing a framework for various possibilities of described embodiments to function together.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present), and B is false (or not present), A is false (or not present), and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

While particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of processing nucleic acid fragments, the method comprising:
providing an input sample comprising nucleic acid fragments, wherein in at least a portion of the nucleic acid fragments each fragment comprises one or more methylated cytosines;
converting unmethylated cytosines of nucleic acid fragments of the input sample to uracils, yielding converted fragments;
copying the converted fragments using a mixture of nucleotides, the mixture comprising a mixture of:
binding moiety-modified cytosines and binding moiety-lacking cytosines;
binding moiety-modified guanines and binding moiety-lacking guanines; or
binding moiety-modified cytosines, binding moiety-lacking cytosines, binding moiety-modified guanines, and binding moiety-lacking guanines;
wherein the copying yields a mixture of binding moiety-modified fragments and unmodified fragments;
binding at least some of the binding moiety-modified fragments to a substrate, yielding bound fragments and unbound supernatant fragments.

2-4. (canceled)

5. The method according to claim 1, wherein the method further comprises separating the bound fragments from the unbound supernatant fragments, yielding the bound fragments enriched for fragments with one or more methylated cytosines.

6. (canceled)

7. The method according to claim 1, wherein the input sample is enriched for targets prior to the converting step.

8. The method according to claim 7, wherein the targets are selected for a methylation assay for cancer, cancer type, cancer tissue of origin, cancer stage, or combinations of the foregoing.

9. (canceled)

10. The method according to claim 1, wherein the input sample comprises DNA isolated from a bodily fluid.

11. The method according to claim 1, wherein the input sample comprises DNA from a cfDNA sample.

12. The method according to claim 1, wherein the input sample comprises fragmented genomic DNA.

13. The method according to claim 1, wherein the converting comprises selectively deaminating the unmethylated cytosines.

14. (canceled)

15. The method according to claim 1, wherein the binding moiety-modified cytosines comprise biotin-modified cytosines, and the binding moiety-modified guanines comprise biotin-modified guanines.

16. (canceled)

17. The method according to claim 1, wherein the substrate comprises beads.

18. The method according to claim 1, wherein the substrate comprises wells.

19-24. (canceled)

25. The method according to claim 1, wherein providing the input sample comprises obtaining from a sample, and including in the input sample, nucleic acid fragments potentially comprising 1 or more CpG sites.

26-30. (canceled)

31. The method according to claim 1, wherein the mixture of nucleotides comprises from 1 to 20 percent binding moiety-modified cytosines with the remainder of the cytosines lacking the binding moiety.

32. The method according to claim 1, wherein the mixture of nucleotides comprises from 2.5 to 10 percent binding moiety-modified cytosines with the remainder of the cytosines lacking the binding moiety.

33. The method according to claim 1, wherein the mixture of nucleotides comprises from 1 to 20 percent binding moiety-modified guanines with the remainder of the guanines lacking the binding moiety.

34. The method according to claim 1, wherein the mixture of nucleotides comprises from 2.5 to 10 percent binding moiety-modified guanines with the remainder of the guanines lacking the binding moiety.

35. The method according to claim 1, wherein the mixture of nucleotides comprises from 1 to 20 percent binding moiety-modified cytosines and guanines with the remainder of the cytosines and guanines lacking the binding moiety.

36. The method according to claim 1, wherein the mixture of nucleotides comprises from 2.5 to 10 percent binding moiety-modified cytosines and guanines with the remainder of the cytosines and guanines lacking the binding moiety.

37. The method according to claim 5, wherein the separating yields bound fragments enriched, relative to the input sample, for informative fragments for a methylation assay.

38. (canceled)

39. The method according to claim 1, further comprising eluting the bound fragments to yield a fragment library enriched, relative to the input sample, for informative fragments for a methylation assay.

40. (canceled)

41. The method according to claim 39, further comprising preparing a sequencing library from the fragment library.

42. The method according to claim 41 further comprising sequencing the sequencing library.

43. The method according to claim 42 wherein the sequencing is performed to a sequencing depth ranging from 5 to 20 million reads.

44-53. (canceled)

54. A composition comprising adenines, thymines, cytosines and guanines wherein the cytosines, guanines, or both cytosines and guanines are included in a mixture of binding moiety-modified nucleotides and binding moiety-lacking nucleotides.

55-61. (canceled)

62. A kit comprising the composition comprising:
the composition according to claim 54; and
instructions for using the composition.

63-68. (canceled)
